SEQUENCE LISTING (16 sequences)

SEQ ID NO: 1 - DNA, 654 bp, synthetic construct
  source 1..654: mol_type = other DNA
atggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtg

SEQ ID NO: 2 - protein, 218 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWV

SEQ ID NO: 3 - DNA, 1113 bp, synthetic construct
  source 1..1113: mol_type = other DNA
atggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - DNA, 1668 bp, synthetic construct
  source 1..1668: mol_type = other DNA
atggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 5 - protein, 371 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 1485 bp, synthetic construct
  source 1..1485: mol_type = other DNA
atggagtttgggctgagctggctttttcttgtggctattttaaaaggtgtccagtgctctagagaggacatccagctgacccagtctccatcctccctgtctgcatctgtaggagacagagtcagcatcacctgcaaggccagtcaggatgtgagtattgctgtagcctggtatcagcagaaaccagggaaagcccctaagctcctgatctactcggcatcctaccggtacactggagtccctgataggttcagtggcagtggatctgggacagatttcactctcaccatcagcagtctgcaacctgaagattttgcagtttattactgtcagcaacattatattactccgctcacgttcggtgctgggaccaaggtggagatcaaacgtttggaaataaagggctctacaagcggctcaggaaaacctggatcaggcgaagggtctacgcaggtccaactgcagcaatctgggtctgagttgaagaagcctggggcctcagtgaaggtttcctgcaaggcttctggatacaccttcacaaactatggaatgaactgggtgaagcaggcccctggacaagggcttaaatggatgggctggataaacacctacactggagagccaacatatactgatgacttcaagggacggtttgccttctccttggacacctctgtcagcacggcatatctccagatcagcagcctaaaggctgacgacactgccgtgtatttctgtgcaagaggggggttcggtagtagctactggtacttcgatgtctggggccaagggtccctggtcaccgtctcctcaccgtacgccattgaagttatgtatcctcctccttacctagacaatgagaagagcaatggaaccattatccatgtgaaagggaaacacctttgtccaagtcccctatttcccggaccttctaagcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgga

SEQ ID NO: 7 - protein, 495 aa, synthetic construct
MEFGLSWLFLVAILKGVQCSREDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRLEIKGSTSGSGKPGSGEGSTQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSPYAIEVMYPPPYLDNEKSNGTIIHVKGKHLCPSPLFPGPSKPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 8 - DNA, 813 bp, synthetic construct
  source 1..813: mol_type = other DNA
atggagtttgggctgagctggctttttcttgtggctattttaaaaggtgtccagtgctctagagaggacatccagctgacccagtctccatcctccctgtctgcatctgtaggagacagagtcagcatcacctgcaaggccagtcaggatgtgagtattgctgtagcctggtatcagcagaaaccagggaaagcccctaagctcctgatctactcggcatcctaccggtacactggagtccctgataggttcagtggcagtggatctgggacagatttcactctcaccatcagcagtctgcaacctgaagattttgcagtttattactgtcagcaacattatattactccgctcacgttcggtgctgggaccaaggtggagatcaaacgtttggaaataaagggctctacaagcggctcaggaaaacctggatcaggcgaagggtctacgcaggtccaactgcagcaatctgggtctgagttgaagaagcctggggcctcagtgaaggtttcctgcaaggcttctggatacaccttcacaaactatggaatgaactgggtgaagcaggcccctggacaagggcttaaatggatgggctggataaacacctacactggagagccaacatatactgatgacttcaagggacggtttgccttctccttggacacctctgtcagcacggcatatctccagatcagcagcctaaaggctgacgacactgccgtgtatttctgtgcaagaggggggttcggtagtagctactggtacttcgatgtctggggccaagggtccctggtcaccgtctcctca

SEQ ID NO: 9 - protein, 271 aa, synthetic construct
MEFGLSWLFLVAILKGVQCSREDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRLEIKGSTSGSGKPGSGEGSTQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 10 - DNA, 3303 bp, synthetic construct
  source 1..3303: mol_type = other DNA
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccatggagtttgggctgagctggctttttcttgtggctattttaaaaggtgtccagtgctctagagaggacatccagctgacccagtctccatcctccctgtctgcatctgtaggagacagagtcagcatcacctgcaaggccagtcaggatgtgagtattgctgtagcctggtatcagcagaaaccagggaaagcccctaagctcctgatctactcggcatcctaccggtacactggagtccctgataggttcagtggcagtggatctgggacagatttcactctcaccatcagcagtctgcaacctgaagattttgcagtttattactgtcagcaacattatattactccgctcacgttcggtgctgggaccaaggtggagatcaaacgtttggaaataaagggctctacaagcggctcaggaaaacctggatcaggcgaagggtctacgcaggtccaactgcagcaatctgggtctgagttgaagaagcctggggcctcagtgaaggtttcctgcaaggcttctggatacaccttcacaaactatggaatgaactgggtgaagcaggcccctggacaagggcttaaatggatgggctggataaacacctacactggagagccaacatatactgatgacttcaagggacggtttgccttctccttggacacctctgtcagcacggcatatctccagatcagcagcctaaaggctgacgacactgccgtgtatttctgtgcaagaggggggttcggtagtagctactggtacttcgatgtctggggccaagggtccctggtcaccgtctcctcaccgtacgccattgaagttatgtatcctcctccttacctagacaatgagaagagcaatggaaccattatccatgtgaaagggaaacacctttgtccaagtcccctatttcccggaccttctaagcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 11 - DNA, 486 bp, synthetic construct
  source 1..486: mol_type = other DNA
atgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 12 - protein, 162 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - DNA, 486 bp, synthetic construct
  source 1..486: mol_type = other DNA
atgaggagcagtccaggcaatatggagcggatagtaatttgtctcatggtaatattcctcggtactctggtacataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagt

SEQ ID NO: 14 - protein, 162 aa, synthetic construct; identical to SEQ ID NO: 12

SEQ ID NO: 15 - DNA, 396 bp, Homo sapiens
  source 1..396: mol_type = other DNA
ggccaggaccggcacatgatccggatgagacagctgatcgacatcgtggaccagctgaagaactacgtgaacgacctggtgcccgagttcctgcctgcccccgaggacgtggaaacaaactgcgagtggagcgccttcagctgcttccagaaggcccagctgaaaagcgccaacaccggcaacaacgagcggatcatcaacgtgtccatcaagaagctgaagcggaagccccccagcaccaacgccggaagaaggcagaagcacagactgacctgccccagctgcgacagctacgagaagaagccccctaaagagttcctggaacggttcaagagcctgctgcagaagatgatccaccagcacctgagcagccggacccacggctctgaggacagc

SEQ ID NO: 16 - protein, 132 aa, Homo sapiens
GQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

(19) United States
(12) Patent Application Publication
(10) Pub. No.: US 2025/0228942 A1
(43) Pub. Date: Jul. 17, 2025

(54) METHODS FOR ACTIVATION AND EXPANSION OF ENGINEERED NATURAL KILLER CELLS AND COMBINATIONS WITH ANTIBODIES

(71) Applicant: BOARD OF REGENTS, THE UNIVERSITY OF TEXAS SYSTEM, Austin, TX (US)

(72) Inventors: Katy REZVANI, Houston, TX (US); Rafet BASAR, Houston, TX (US); Sunil ACHARYA, Houston, TX (US); Nadima UPRETY, Houston, TX (US); May DAHER, Houston, TX (US); Elizabeth SHPALL, Houston, TX (US); David MARIN COSTA, Houston, TX (US)

(73) Assignee: BOARD OF REGENTS, THE UNIVERSITY OF TEXAS SYSTEM, Austin, TX (US)

(21) Appl. No.: 18/850,488

(22) PCT Filed: Apr. 7, 2023
(86) PCT No.: PCT/US2023/065493

§ 371 Date: Sep. 24, 2024

Related U.S. Application Data
(60) Provisional application No. 63/415,517, filed on Oct. 12, 2022.
(60) Provisional application No. 63/348,259, filed on Jun. 2, 2022.

(30) Foreign Application Priority Data
Apr. 7, 2022 (WO) PCT/US2022/023920

(51) Int. Cl. A61K 40/15; A61K 39/395; A61K 40/31; A61K 40/42; A61P 35/00; C12N 5/0783

(52) U.S. Cl. CPC A61K 40/15; A61K 39/39558; A61K 40/31; A61K 40/42; A61K 40/4224; A61P 35/00; C12N 5/0646; C12N 2501/2302; C12N 2501/2312; C12N 2501/2315; C12N 2501/2318; C12N 2510/00

(57) ABSTRACT

Embodiments of the disclosure concern methods and compositions related to preparation and use of combinatorial immunotherapies. In specific embodiments, compositions comprising engineered NK cells prepared in a particular manner also include certain antibodies. These compositions are utilized for treatment, such as for cancer treatment. In particular embodiments, the compositions include complexes of the engineered NK cells and the antibodies in which the antibody is bound to the engineered NK cells and may also bind to another antigen, such as on a cancer cell.

### CROSS-REFERENCE TO RELATED APPLICATIONS

This application claims priority to International Application Serial No. PCT/US2022/023920, filed Apr. 7, 2022, U.S. Provisional Patent Application Ser. No. 63/348,259, filed Jun. 2, 2022, and U.S. Provisional Patent Application Ser. No. 63/415,517, filed Oct. 12, 2022, each of which is incorporated by reference herein in their entirety.

### SEQUENCE LISTING

The instant application contains a Sequence Listing which has been submitted in ST26 format and is hereby incorporated by reference in its entirety. Said ST26 copy, created on Apr. 3, 2023, is named MDAC_1330WO_Sequence_Listing.xml and is 26,915 bytes in size.

### TECHNICAL FIELD

Embodiments of the disclosure include at least the fields of cell biology, molecular biology, immunology, and medicine, including cancer medicine.

### BACKGROUND

In the field of natural killer (NK) cells as a therapy, certain barriers impede their use, including the requirement for ex vivo expansion because of limited numbers, in addition to their insufficient persistence that impacts their efficacy as an anti-cancer therapy. Cytokine stimulation constitutes an important signal to enhance the functional competency of NK cells to respond to tumor target cells. Furthermore, pre-activation of NK cells overnight with the combination of IL-18, IL-15, and IL-12 has been shown to generates long-lived, memory-like NK cells with enhanced cytokine production upon re-stimulation (Leong et al., 2014). However, procedures for using pre-activated NK cells from peripheral blood (PB) in the absence of ex vivo expansion require cumbersome procedures to generate sufficient amounts for clinical use. In addition, there are other obstacles related to deficient cancer recognition and poor NK cell activation. Thus, there is an unmet need for improved strategies to generate highly functional NK cells for therapeutic applications and that are particularly efficient at cancer antigen targeting.

### BRIEF SUMMARY

Embodiments of the disclosure concerns methods and compositions related to immunotherapy for a medical condition. The immunotherapy may be utilized for any medical condition in which targeting of cells in need of destruction is clinically useful and for which there are specific antigens on the targeted cells for which antibodies and/or cells may be specific. In specific embodiments, the immunotherapy comprises, consists of, or consists essentially of adoptive cell therapy and antibodies that are configured to bind to the cells.

In some embodiments, provided herein are compositions comprising (i) one or more engineered natural killer (NK) cells comprising one or more transgenes; and (ii) one or more antibody molecules comprising Amivantamab, Margetuximab, and/or Imgatuzumab. In some embodiments, an engineered NK cell comes from a population that is or is not expanded. In some embodiments, an engineered NK cell comes from a population that is expanded. In some embodiments, an engineered NK cell comes from a population that is or is not pre-activated. In some embodiments, an engineered NK cell comes from a population that is pre-activated.

In some embodiments, a composition provided herein is further defined as a complex between the engineered NK cell and Margetuximab, Amivantamab, and/or Imgatuzumab, through binding of the Fc region of the antibody to the engineered NK cell. In some embodiments, a complex further comprises one or more antigen binding domain(s) of the one or more antibody bound to its target antigen.

In some embodiments, one or more antibody target antigens are Epidermal Growth Factor Receptor (EGFR), Epidermal Growth Factor Receptor-2 (HER2/EGFR-2), and/or tyrosine-protein kinase Met (c-Met). In some embodiments, engineered NK cells are derived from NK cells or NK cell precursors obtained from cord blood (CB) mononuclear cells, CB hematopoietic stem cells, peripheral blood (PB) NK cells, bone marrow, stem cells, iPSCs, NK cell lines, or a combination thereof. In some embodiments, engineered NK cells are derived from primary NK cells, and are not derived from stem cells and/or induced pluripotent stem cells (iPSCs). In some embodiments, NK cells are sourced from cord blood, and wherein the source of the CB mononuclear cells and/or CB hematopoietic stem cells is CB from 1 donor or pooled from 2 or more individual CB units. In some embodiments, engineered NK cells are modified to comprise one or more transgenes for expression of one or more heterologous proteins.

In some embodiments, one or more heterologous proteins comprises one or more engineered receptors, and/or one or more cytokines. In some embodiments, one or more engineered receptor is a chimeric antigen receptor (CAR), a T cell receptor (TCR), receptors recognizing the FC portion of Ig (FC recognition receptor), a chemokine receptor, a homing receptor, a chimeric cytokine receptor, or any combination thereof. In some embodiments, an FC recognition receptor comprises CD16, CD32, and/or CD64, and/or wherein the engineered NK cells express one or more transgenes encoding one or more receptors to enhance their binding to an antibody. In some embodiments, an engineered receptor comprises a TCR and/or a CAR. In some embodiments, the engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

In some embodiments, an engineered antigen receptor targets CD70 and/or TROP2. In some embodiments, an antigen receptor targeting CD70 comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5. In some embodiments, an engineered NK cell comprises a construct that comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4. In some embodiments, an antigen receptor targeting TROP2 comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9. In some embodiments, an engineered NK cell comprises a construct comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10.

In some embodiments, an engineered NK cell comprises one or more heterologous cytokine comprising IL-2, IL-4, IL-7, IL-12, IL-15, IL-18, IL-21 and/or IL-23. In some embodiments, a cytokine comprises, consists, or consists essentially of IL-15 encoded by and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12. In some embodiments, a cytokine comprises, consists, or consists essentially of IL-21 encoded by and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16.

In some embodiments, an engineered NK cells express one or more transgenes encoding one or more suicide genes. In some embodiments, an engineered NK cells comprise one or more engineered mutations in an endogenous gene. In some embodiments, an engineered mutation comprises a CISH, CD38, Glucocorticoid Receptor, and/or TGFBR2 gene mutation.

In some embodiments, complexing of one or more antibody molecules and NK cells comprises incubation of the NK cells and one or more antibody for at least or about 1 hour prior to washing of the composition to remove unbound antibodies. In some embodiments, washing comprises washing at least twice with a suitable solution. In some embodiments, a suitable solution comprises PBS. In some embodiments, washing comprises washing for at least or about 1 minute, 2 minutes, 3 minutes, 4 minutes, 5 minutes, 6 minutes, 7 minutes, 8 minutes, 9 minutes, 10 minutes, or longer than 10 minutes. In some embodiments, washing comprises washing for at least or about 5 minutes.

In some embodiments, one or more antibody molecules are incubated with NK cells for formation of a complex at a concentration of about 1 to about 1000 μg/ml final concentration, about 1 μg/ml final concentration, about 10 μg/ml final concentration, about 50 μg/ml final concentration, or about 100 μg/ml final concentration. In some embodiments, one or more antibody and NK cells are incubated in Click's/RPMI media. In some embodiments, incubating and washing conditions for complexing of the engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing. In some embodiments, relative to the amount of antibodies complexed to the engineered NK cells measured about 1 hour after incubation and washing, at least about 5%, 6%, 7%, 8%, 9%, 10%, 11%, 12%, or greater than 12% of the antibodies are bound to the NK cell surface at least 3 days after incubation and washing. In some embodiments, an relative to the amount of antibodies complexed to the engineered NK cells measured about 1 hour after incubation and washing, at least about 1%, 2%, 3%, 4%, 5%, 6%, 7%, 8%, 9%, 10%, or greater than 10% of the antibodies are bound to the NK cell surface at least 5 days after incubation and washing. In some embodiments, relative to the amount of antibodies complexed to the engineered NK cells measured about 1 hour after incubation and washing, at least about 1%, 2%, 3%, 4%, 5%, 6%, 7%, 8%, or greater than 8% of the antibodies are bound to the NK cell surface at least 7 days after incubation and washing. In some embodiments, relative to the amount of antibodies complexed to the engineered NK cells measured about 1 hour after incubation and washing, at least about 0.5%, 1%, 2%, 3%, 4%, 5%, or greater than 5% of the antibodies are bound to the NK cell surface at least 12 days after incubation and washing.

In some embodiments, a composition comprising engineered NK cells and antibodies has not been frozen, is cryopreserved, or was thawed from cryopreservation. In some embodiments, a composition comprising engineered NK cells and antibodies is cryopreserved. In some embodiments, a composition is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody. In some embodiments, relative to the amount of antibodies complexed to non-cryopreserved engineered NK cells measured about 1 hour after incubation and washing, at least about 5%, 10%, 20%, 30%, 40%, 50%, or 60%, or any range derivable therein, of the antibodies are bound to the NK cell surface after thawing from cryopreservation. In some embodiments, engineered NK cells are deactivated prior to cryopreservation. In some embodiments, an NK cell deactivating agent comprises a kinase inhibitor. In some embodiments, an NK cell deactivating agent comprises, consists, or consists essentially of Dasatinib.

In some embodiments, a composition comprising engineered NK cells and antibodies is produced in vivo. In some embodiments, production of the composition in vivo comprises loading the engineered NK cells in vivo with antibody by administering the engineered NK cells within about 1 hour of administration of an antibody. In some embodiments, a one or more antibody comprises or consists of Amivantamab. In some embodiments, a one or more antibody comprises or consists of Margetuximab. In some embodiments, a one or more antibody comprises or consists of Imgatuzumab.

Also provided herein are methods of making compositions described herein. In some embodiments, a method of producing a composition comprises the steps of: (i) optionally expanding engineered NK cells in a culture comprising an effective amount of: (a) a cytokine selected from the group consisting of IL-2, IL-15, IL-18, IL-21 and a combination thereof; and (b) antigen presenting cells/feeders, fragments of antigen presenting cells/feeders, or NK cell-activating beads; and (ii) providing one or more antibody molecules comprising Amivantamab, Margetuximab, and/or Imgatuzumab to the engineered NK cells.

In some embodiments, a method comprises a pre-activating step prior to and/or after the expanding step, wherein the engineered NK cells are pre-activated in a culture comprising an effective concentration of one or more of IL-2, IL-12, IL-15, and IL-18. In some embodiments, a culture comprises an effective concentration of IL-12, IL-15, and IL-18. In some embodiments, a providing step is further defined as incubating the engineered NK cells with the antibody molecules for a specific duration of time, combining the engineered NK cells and the antibody molecules just prior to infusion, or combining the engineered NK cells and the antibody in vivo in a subject through temporally coordinated administration. In some embodiments, a duration of time is about 5 minutes to about 24 hours or more. In some embodiments, a duration of time is at most, at least, or about 1 hour. In some embodiments, a culture for the pre-activating step comprises IL-18 and/or IL-15 at a concentration of 0.1-1000 ng/ml, 1-1000 ng/mL, or about 10 ng/ml. In some embodiments, a culture for the pre-activating step comprises IL-12 at a concentration of 0.1-1000 ng/mL, 1-1000 ng/mL, or about 10 ng/mL. In some embodiments, methods further comprise one or more washing steps of the pre-activated engineered NK cells prior to and/or after the expanding step. In some embodiments, engineered NK cells are activated at least twice or more during the expansion step with IL-12, IL-15, IL-18, IL-2 or any combination thereof. In some embodiments, an NK cell expanding step is for 5-60 days, 12-16 days, or 18-24 days. In some embodiments, expansion culture further comprises IL-2. In some embodiments, the IL-2 is present at a concentration of 10-500 U/mL, 100-300 U/mL, or about 200 U/mL. In some embodiments, the IL-2 is replenished in the expansion culture every 2-3 days.

In some embodiments, methods described herein comprise incubating conditions for complexing of engineered NK cells and one or more antibody, such that at least or about 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any range derivable therein, of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days, or any range derivable therein following incubation and washing. In some embodiments, relative to the amount of antibodies complexed to the engineered NK cells measured about 1 hour after incubation and washing, at least about 5%, 6%, 7%, 8%, 9%, 10%, 11%, 12%, or greater than 12% of the antibodies are bound to the NK cell surface at least 3 days after incubation and washing. In some embodiments, relative to the amount of antibodies complexed to the engineered NK cells measured about 1 hour after incubation and washing, at least about 1%, 2%, 3%, 4%, 5%, 6%, 7%, 8%, 9%, 10%, or greater than 10% of the antibodies are bound to the NK cell surface at least 5 days after incubation and washing. In some embodiments, relative to the amount of antibodies complexed to the engineered NK cells measured about 1 hour after incubation and washing, at least about 1%, 2%, 3%, 4%, 5%, 6%, 7%, 8%, or greater than 8% of the antibodies are bound to the NK cell surface at least 7 days after incubation and washing. In some embodiments, relative to the amount of antibodies complexed to the engineered NK cells measured about 1 hour after incubation and washing, at least about 0.5%, 1%, 2%, 3%, 4%, 5%, or greater than 5% of the antibodies are bound to the NK cell surface at least 12 days after incubation and washing.

In some embodiments, a method of preparing a composition comprises not freezing, or alternatively cryopreserving and/or thawing from cryopreservation the composition. In some embodiments, NK cells provided with one or more antibodies are not frozen, are cryopreserved, or are thawed from cryopreservation. In some embodiments, the NK cells provided with one or more antibodies are thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody. In some embodiments, relative to the amount of antibodies complexed to non-cryopreserved engineered NK cells measured about 1 hour after incubation and washing, at least about 5%, 10%, 20%, 30%, 40%, 50%, or 60%, or greater than 60% of the antibodies are bound to the NK cell surface after thawing from cryopreservation.

Also provided herein are methods of treating a disease or disorder in a subject comprising administering a therapeutically effective amount of any one or more of the compositions described herein to a subject. In some embodiments, a disease or disorder is cancer, inflammation, graft versus host disease, transplant rejection, an autoimmune disorder, an immunodeficiency disease, a B cell malignancy, or an infection. In some embodiments, a cancer comprises a hematological cancer or a solid tumor. In some embodiments, a cancer comprises pancreatic cancer, colorectal cancer, kidney cancer, brain cancer, breast cancer, renal cancer, and/or myeloma. In some embodiments, engineered NK cells are allogeneic with respect to the subject. In some embodiments, engineered NK cells are autologous with respect to the subject. In some embodiments, the subject is a human. In some embodiments, methods of treatment further comprising administering at least a second therapeutic agent to the subject. In some embodiments, the at least a second therapeutic agent is a therapeutically effective amount of one or more anti-cancer agents, one or more immunomodulatory agents, and/or one or more immunosuppressive agents. In some embodiments, the anti-cancer agent is chemotherapy, radiotherapy, gene therapy, surgery, hormonal therapy, anti-angiogenic therapy or immunotherapy. In some embodiments, the immunosuppressive agent is a calcineurin inhibitor, an mTOR inhibitor, an antibody, a chemotherapeutic agent irradiation, a chemokine, an interleukins or an inhibitor of a chemokine or an interleukin. In some embodiments, the composition and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion. In some embodiments, the second therapeutic agent is an antibody.

In some embodiments, provided herein are methods of treating a subject with cancer with an engineered NK cell, comprising: a) optionally stimulating a population of engineered NK cells by pre-activation with a cytokine cocktail; b) optionally expanding the engineered NK cells; and ci) loading the engineered NK cells ex vivo with one or more antibody and then administering the loaded engineered NK cells to the subject, and/or cii) loading the engineered NK cells in vivo with one or more antibody by administering the engineered NK cells within 1 hour of administration of an antibody; wherein the engineered NK cell comprises one or more transgenes, wherein the loading comprises exposure of engineered NK cells to the one or more antibody for an amount of time effective for binding of the antibody to the engineered NK cell surface, and wherein the one or more antibody comprises or consists of Amivantamab, Margetuximab, and/or Imgatuzumab. In some embodiments, methods comprise administering a composition comprising engineered NK cells that were loaded ex vivo, followed by administering additional antibody to a subject at a later time point for additional in vivo loading of the NK cells with antibody. In some embodiments, loading an engineered NK cells comprises exposure of the engineered NK cells to the one or more antibody for greater than or equal to about 1 hour, or less than or equal to about 1 hour. In some embodiments, engineered NK cells remain loaded with antibody for at least about or about 1 hour, 4 hours, 8 hours, 12 hours, 1 day, 2 days, 3 days, 4 days, 5 days, 6 days, 7 days, 8 days, 9 days, 10 days, 11 days, or 12 days. In some embodiments one or more antibody is bound to the engineered NK cell through interaction with a CD16 surface protein. In some embodiments, engineered NK cells are derived from cord blood. In some embodiments, engineered NK cells are activated ex-vivo. In some embodiments, engineered NK cells are pre-activated with IL-18, IL-15, and IL-12.

In some embodiments, methods of treatment comprise use of an NK cell comprising one or more transgenes comprising an engineered receptor. In some embodiments, an engineered receptor comprises a chimeric antigen receptor (CAR), a T cell receptor, chemokine receptor, chimeric cytokine receptor, or any combination thereof. In some embodiments, an engineered receptor comprises a CAR or a TCR. In some embodiments, an engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof. In some embodiments, an engineered antigen receptor targets CD70 and/or TROP2. In some embodiments, an antigen receptor targeting CD70 comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5. In some embodiments, an engineered NK cell comprises a construct that comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4. In some embodiments, an antigen receptor targeting TROP2 comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9. In some embodiments, an engineered NK cell comprises a construct comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10. In some embodiments, an NK cell comprises one or more transgenes comprising a cytokine (e.g., a heterologous cytokine). In some embodiments, a one or more heterologous cytokine comprises IL-2, IL-4, IL-7, IL-12, IL-15, IL-18, IL-21 and/or IL-23. In some embodiments, a heterologous cytokine comprises IL-2, IL-12, IL-15, IL-18, and/or IL-21. In some embodiments, a cytokine comprises, consists, or consists essentially of IL-15 encoded by and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12. In some embodiments, a cytokine comprises, consists, or consists essentially of IL-21 encoded by and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16. In some embodiments, an engineered NK cells comprise on or more engineered mutations in an endogenous gene. In some embodiments, the one or more engineered mutation in an endogenous gene comprises a CISH, CD38, Glucocorticoid Receptor, and/or TGFBR2 gene mutation.

Also provided herein are compositions comprising (i) one or more engineered natural killer (NK) cells comprising one or more transgenes; and (ii) one or more antibodies comprising antibody IPH61. In some embodiments, one or more antibodies targets at least CD123 and NKp46. In some embodiments, a composition is further defined as a complex between the engineered NK cell IPH61 through binding of the Fc region of the antibody to the engineered NK cell and/or binding of the NKp46 targeting region of the antibody to the engineered NK cell. In some embodiments, a complex further comprises one or more antigen binding domain(s) of the one or more antibody bound to its target antigen. In some embodiments, an engineered NK cells are derived from NK cells or NK cell precursors obtained from cord blood (CB) mononuclear cells, CB hematopoietic stem cells, peripheral blood (PB) NK cells, bone marrow, stem cells, iPSCs, NK cell lines, or a combination thereof. In some embodiments, an engineered NK cell is a primary NK cell (e.g., an NK cell not derived from a stem cell, iPSC, etc.).

In some embodiments, compositions comprise an engineered NK cell modified to comprise one or more transgenes for expression of one or more heterologous proteins. In some embodiments, one or more heterologous proteins comprises one or more engineered receptors, and/or one or more cytokines. In some embodiments, one or more engineered receptor is a chimeric antigen receptor (CAR), a T cell receptor (TCR), receptors recognizing the FC portion of Ig (FC recognition receptor), a chemokine receptor, a homing receptor, a chimeric cytokine receptor, or any combination thereof. In some embodiments, an FC recognition receptor comprises CD16, CD32, and/or CD64, and/or wherein the engineered NK cells express one or more transgenes encoding one or more receptors and/or NK cell surface antigens to enhance NK cell binding to an antibody. In some embodiments, an engineered NK cell surface antigen and/or receptor is CD16, CD32, CS1, CD56, CD64, NKG2D, NKG2C, DNAM, 2B4, CD2, an NCR, NKp30, NKp44, NKp46, or KIR. In some embodiments, an engineered receptor comprises a TCR and/or a CAR. In some embodiments, an engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof. In some embodiments, engineered antigen receptor targets CD70 and/or TROP2. In some embodiments, an antigen receptor targeting CD70 comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5. In some embodiments, engineered NK cell comprises a construct that comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4. In some embodiments, an antigen receptor targeting TROP2 comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9. In some embodiments, an engineered NK cell comprises a construct that comprises, consists, or consists essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10. In some embodiments, one or more heterologous cytokine is IL-2, IL-4, IL-7, IL-12, IL-15, IL-18, IL-21 and/or IL-23. In some embodiments, a cytokine comprises, consists of, or consists essentially of IL-15 encoded by a sequence comprising, consisting of, or consisting essentially of at least or about, and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12. In some embodiments, a cytokine comprises, consists of, or consists essentially of IL-21 encoded by a sequence comprising, consisting of, or consisting essentially of at least or about, and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 13-16. In some embodiments, engineered NK cells express one or more transgenes encoding one or more suicide genes. In some embodiments, engineered NK cells comprise one or more engineered mutations in an endogenous gene. In some embodiments, one or more engineered mutations comprises a CISH, CD38, Glucocorticoid Receptor, and/or TGFBR2 gene mutation. In some embodiments, complexing of the one or more antibodies comprising IPH61 and NK cells comprises incubation of the NK cells and one or more antibody for at least or about 1 hour prior to washing of the composition to remove unbound antibodies.

In some embodiments, compositions comprise one or more antibody molecules incubated with NK cells for formation of a complex at a concentration of about 1 to about 1000 μg/ml final concentration, about 1 μg/ml final concentration, about 10 μg/ml final concentration, about 50 μg/ml final concentration, or about 100 μg/ml final concentration. In some embodiments, the antibody and NK cells are incubated in Click's/RPMI media. In some embodiments, incubating and washing conditions for complexing of the engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing. In some embodiments, the composition has not been frozen, is cryopreserved, or was thawed from cryopreservation. In some embodiments, the composition is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody. In some embodiments, relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation. In some embodiments, the engineered NK cells are deactivated prior to cryopreservation. In some embodiments, the deactivating agent comprises a kinase inhibitor. In some embodiments, the deactivating agent comprises Dasatinib. In some embodiments, the composition is produced in vivo. In some embodiments, production of the composition in vivo comprises loading the engineered NK cells in vivo with one or more antibody by administering the engineered NK cells within about 1 hour of administration of an antibody.

Also provided herein are methods of producing compositions as described herein, comprising the steps of: (i) optionally expanding engineered NK cells in a culture comprising an effective amount of: (a) a cytokine selected from the group consisting of IL-2, IL-15, IL-18, IL-21 and a combination thereof; and (b) antigen presenting cells/feeders, fragments of antigen presenting cells/feeders, or NK cell-activating beads; and (ii) providing one or more antibody molecules comprising IPH61 to the engineered NK cells. In some embodiments, a method comprises a pre-activating step prior to and/or after the expanding step, wherein the engineered NK cells are pre-activated in a culture comprising an effective concentration of one or more of IL-2, IL-12, IL-15, and IL-18. In some embodiments, the culture comprises an effective concentration of IL-12, IL-15, and IL-18. In some embodiments, the providing step is further defined as incubating the engineered NK cells with the antibody molecules for a specific duration of time, combining the engineered NK cells and the antibody molecules just prior to infusion, or combining the engineered NK cells and the antibody in vivo in a subject through temporally coordinated administration. In some embodiments, the duration of time is about 5 minutes to about 24 hours or more. In some embodiments, the duration of time is about 1 hour. In some embodiments, the culture for the pre-activating step comprises IL-18 and/or IL-15 at a concentration of 0.1-1000 ng/ml, 1-1000 ng/mL, or about 10 ng/ml. In some embodiments, the culture for the pre-activating step comprises IL-12 at a concentration of 0.1-1000 ng/ml, 1-1000 ng/ml, or about 10 ng/mL. In some embodiments, expanding is for 5-60 days, 12-16 days, or 18-24 days. In some embodiments, the expansion culture further comprises IL-2. In some embodiments, IL-2 is present at a concentration of 10-500 U/mL, 100-300 U/mL, or about 200 U/mL.

In some embodiments, incubating conditions for complexing of engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing. In some embodiments, NK cells provided with antibodies are not frozen, are cryopreserved, or are thawed from cryopreservation. In some embodiments, NK cells provided with antibodies are thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody. In some embodiments, relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

Also provided herein are methods of treating a disease or disorder in a subject, comprising administering a therapeutically effective amount of a composition comprising NK cells and one or more antibodies comprising IPH61, by administration to the subject. In some embodiments, the disease or disorder is cancer, inflammation, graft versus host disease, transplant rejection, an autoimmune disorder, an immunodeficiency disease, a B cell malignancy, or an infection. In some embodiments, the cancer comprises a hematological cancer or a solid tumor. In some embodiments, the cancer comprises acute myeloid leukemia, blastic plasmacytoid dendritic cell neoplasm, acute lymphoblastic leukemia, hairy cell leukemia, and/or systemic mastocytosis. In some embodiments, the engineered NK cells are allogeneic with respect to the subject. In some embodiments, the engineered NK cells are autologous with respect to the subject. In some embodiments, the subject is a human. In some embodiments, a method of treatment further comprises administering at least a second therapeutic agent to the subject. In some embodiments, at least a second therapeutic agent is a therapeutically effective amount of one or more anti-cancer agents, one or more immunomodulatory agents, and/or one or more immunosuppressive agents.

Also provided herein are compositions comprising (i) one or more engineered natural killer (NK) cells comprising one or more transgenes; and (ii) one or more antibody molecules, wherein the one or more transgenes comprise a CD70 targeting CAR comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5, and/or wherein the one or more transgenes comprise a TROP2 targeting CAR comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9. In some embodiments, the engineered NK cell comes from a population that is or is not expanded. In some embodiments, the engineered NK cell comes from a population that is expanded. In some embodiments, the engineered NK cell comes from a population that is or is not pre-activated. In some embodiments, the engineered NK cell comes from a population that is pre-activated.

In some embodiments, a composition is further defined as a complex between the engineered NK cell and one or more antibodies. In some embodiments, the complex further comprises one or more antigen binding domain(s) of the one or more antibody bound to its target antigen. In some embodiments, an antibody targets a cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof. In some embodiments, one or more antibody targets Epidermal Growth Factor Receptor (EGFR), Epidermal Growth Factor Receptor-2 (HER2/EGFR-2), NKp46, CD123, and/or tyrosine-protein kinase Met (c-Met).

In some embodiments, engineered NK cells are derived from NK cells or NK cell precursors obtained from cord blood (CB) mononuclear cells, CB hematopoietic stem cells, peripheral blood (PB) NK cells, bone marrow, stem cells, iPSCs, NK cell lines, or a combination thereof. In some embodiments, engineered NK cells are derived from primary NK cells, and are not derived from stem cells and/or induced pluripotent stem cells (iPSCs). In some embodiments, NK cells are sourced from cord blood, and wherein the source of the CB mononuclear cells and/or CB hematopoietic stem cells is CB from 1 donor or pooled from 2 or more individual CB units.

In some embodiments, an engineered NK cell is further modified to comprise one or more additional transgenes for expression of one or more heterologous proteins. In some embodiments, the one or more heterologous proteins comprises one or more engineered receptors, and/or one or more cytokines. In some embodiments, the one or more engineered receptor is a chimeric antigen receptor (CAR), a T cell receptor (TCR), receptors recognizing the FC portion of Ig (FC recognition receptor), a chemokine receptor, a homing receptor, a chimeric cytokine receptor, or any combination thereof. In some embodiments, the FC recognition receptor comprises CD16, CD32, and/or CD64, and/or wherein the engineered NK cells express one or more transgenes encoding one or more receptors to enhance their binding to an antibody. In some embodiments, the engineered receptor comprises a TCR and/or a CAR. In some embodiments, an engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

In some embodiments, an engineered NK cell comprises a construct comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4. In some embodiments, an engineered NK cell comprises a construct comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6, 8, or 10.

In some embodiments, an NK cell is engineered to express one or more heterologous cytokines. In some embodiments, one or more heterologous cytokine comprises IL-2, IL-4, IL-7, IL-12, IL-15, IL-18, IL-21 and/or IL-23. In some embodiments, a cytokine comprises, consists of, or consists essentially of IL-15 encoded by a sequence comprising, consisting of, or consisting essentially of at least or about, and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 11 or 12. In some embodiments, a cytokine comprises, consists of, or consists essentially of IL-21 encoded by a sequence comprising, consisting of, or consisting essentially of at least or about, and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16. In some embodiments, an engineered NK cells express one or more transgenes encoding one or more suicide genes. In some embodiments, an engineered NK cells comprise one or more engineered mutations in an endogenous gene. In some embodiments, an engineered mutation comprises a CISH, CD38, Glucocorticoid Receptor, and/or TGFBR2 gene mutation.

In some embodiments, complexing of the one or more antibody molecules and NK cells comprises incubation of the NK cells and one or more antibody for at least or about 1 hour prior to washing of the composition to remove unbound antibodies. In some embodiments, one or more antibody molecules are incubated with NK cells for formation of a complex at a concentration of about 1 to about 1000 μg/ml final concentration, about 1 μg/ml final concentration, about 10 μg/ml final concentration, about 50 μg/ml final concentration, or about 100 g/ml final concentration. In some embodiments, antibody and NK cells are incubated in Click's/RPMI media. In some embodiments, incubating and washing conditions for complexing of the engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing. In some embodiments, relative to the amount of antibodies complexed to the engineered NK cells measured about 1 hour after incubation and washing, at least about 9% of the antibodies are bound to the NK cell surface at least 3 days after incubation and washing. In some embodiments, relative to the amount of antibodies complexed to the engineered NK cells measured about 1 hour after incubation and washing, at least about 6% of the antibodies are bound to the NK cell surface at least 5 days after incubation and washing. In some embodiments, relative to the amount of antibodies complexed to the engineered NK cells measured about 1 hour after incubation and washing, at least about 4% of the antibodies are bound to the NK cell surface at least 7 days after incubation and washing. In some embodiments, relative to the amount of antibodies complexed to the engineered NK cells measured about 1 hour after incubation and washing, at least about 3% of the antibodies are bound to the NK cell surface at least 12 days after incubation and washing. In some embodiments, the composition has not been frozen, is cryopreserved, or was thawed from cryopreservation. In some embodiments, the composition is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody. In some embodiments, relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation. In some embodiments, the engineered NK cells are deactivated prior to cryopreservation. In some embodiments, the deactivating agent comprises a kinase inhibitor. In some embodiments, the deactivating agent comprises Dasatinib. In some embodiments, the composition is produced in vivo. In some embodiments, production of the composition in vivo comprises loading the engineered NK cells in vivo with antibody by administering the engineered NK cells within about 1 hour of administration of an antibody. In some embodiments, the one or more antibody comprises or consists of Amivantamab. In some embodiments, the one or more antibody comprises or consists of Margetuximab. In some embodiments, the one or more antibody comprises or consists of Imgatuzumab. In some embodiments, the one or more antibody comprises or consists of IPH61.

Also provided herein are methods of producing compositions comprising the steps of: (i) optionally expanding engineered NK cells in a culture comprising an effective amount of: (a) a cytokine selected from the group consisting of IL-2, IL-15, IL-18, IL-21 and a combination thereof; and (b) antigen presenting cells/feeders, fragments of antigen presenting cells/feeders, or NK cell-activating beads; and (ii) providing one or more antibody molecules to the engineered NK cells. In some embodiments, the method comprises a pre-activating step prior to and/or after the expanding step, wherein the engineered NK cells are pre-activated in a culture comprising an effective concentration of one or more of IL-2, IL-12, IL-15, and IL-18. In some embodiments, the culture comprises an effective concentration of IL-12, IL-15, and IL-18. In some embodiments, the providing step is further defined as incubating the engineered NK cells with the antibody molecules for a specific duration of time, combining the engineered NK cells and the antibody molecules just prior to infusion, or combining the engineered NK cells and the antibody in vivo in a subject through temporally coordinated administration. In some embodiments, the duration of time is about 5 minutes to about 24 hours or more. In some embodiments, the duration of time is about 1 hour. In some embodiments, the culture for the pre-activating step comprises IL-18 and/or IL-15 at a concentration of 0.1-1000 ng/ml, 1-1000 ng/ml, or about 10 ng/ml. In some embodiments, the culture for the pre-activating step comprises IL-12 at a concentration of 0.1-1000 ng/mL, 1-1000 ng/mL, or about 10 ng/mL. In some embodiments, methods further comprise one or more washing steps of the pre-activated engineered NK cells prior to and/or after the expanding step. In some embodiments, engineered NK cells are activated at least twice or more during the expansion step with IL-12, IL-15, IL-18, IL-2 or any combination thereof. In some embodiments, expanding is for 5-60 days, 12-16 days, or 18-24 days. In some embodiments, the expansion culture further comprises IL-2. In some embodiments, the IL-2 is present at a concentration of 10-500 U/mL, 100-300 U/mL, or about 200 U/mL. In some embodiments, the IL-2 is replenished in the expansion culture every 2-3 days.

In some embodiments, incubating conditions for complexing of the engineered NK cells and one or more antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing. In some embodiments, relative to the amount of antibodies complexed to the engineered NK cells measured about 1 hour after incubation and washing, at least about 9% of the antibodies are bound to the NK cell surface at least 3 days after incubation and washing. In some embodiments, relative to the amount of antibodies complexed to the engineered NK cells measured about 1 hour after incubation and washing, at least about 6% of the antibodies are bound to the NK cell surface at least 5 days after incubation and washing. In some embodiments, relative to the amount of antibodies complexed to the engineered NK cells measured about 1 hour after incubation and washing, at least about 4% of the antibodies are bound to the NK cell surface at least 7 days after incubation and washing. In some embodiments, relative to the amount of antibodies complexed to the engineered NK cells measured about 1 hour after incubation and washing, at least about 3% of the antibodies are bound to the NK cell surface at least 12 days after incubation and washing. In some embodiments, the NK cells provided with one or more antibodies are not frozen, are cryopreserved, or are thawed from cryopreservation. In some embodiments, the NK cells provided with one or more antibodies are thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody. In some embodiments, relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

Also provided herein are methods of treating a disease or disorder in a subject, comprising administering a therapeutically effective amount of any one or more of the compositions provided herein to the subject. In some embodiments, the disease or disorder is cancer, inflammation, graft versus host disease, transplant rejection, an autoimmune disorder, an immunodeficiency disease, a B cell malignancy, or an infection. In some embodiments, the cancer comprises a hematological cancer or a solid tumor. In some embodiments, the cancer comprises pancreatic cancer, colorectal cancer, kidney cancer, brain cancer, breast cancer, renal cancer, and/or myeloma. In some embodiments, the cancer comprises acute myeloid leukemia, blastic plasmacytoid dendritic cell neoplasm, acute lymphoblastic leukemia, hairy cell leukemia, and/or systemic mastocytosis. In some embodiments, the engineered NK cells are allogeneic with respect to the subject. In some embodiments, the engineered NK cells are autologous with respect to the subject. In some embodiments, subject is a human. In some embodiments, methods of treatment further comprise administering at least a second therapeutic agent to the subject. In some embodiments, at least a second therapeutic agent is a therapeutically effective amount of one or more anti-cancer agents, one or more immunomodulatory agents, and/or one or more immunosuppressive agents. In some embodiments, the at least a second therapeutic agent is the same antibody and/or targets the same antigen as the antibody that was provided previously in the composition comprising an antibody loaded NK cell. In some embodiments, the anti-cancer agent is chemotherapy, radiotherapy, gene therapy, surgery, hormonal therapy, anti-angiogenic therapy or immunotherapy. In some embodiments, the immunosuppressive agent is a calcineurin inhibitor, an mTOR inhibitor, an antibody, a chemotherapeutic agent irradiation, a chemokine, an interleukins or an inhibitor of a chemokine or an interleukin. In some embodiments, the composition and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion. In some embodiments, the second therapeutic agent is an antibody.

Also provided herein are methods of treating a subject with cancer with an engineered NK cell, the method comprising: a) optionally stimulating a population of engineered NK cells by pre-activation with a cytokine cocktail; b) optionally expanding the engineered NK cells; and ci) loading the engineered NK cells ex vivo with one or more antibody and then administering the loaded engineered NK cells to the subject, or cii) loading the engineered NK cells in vivo with one or more antibody by administering the engineered NK cells within 1 hour of administration of an antibody; wherein the engineered NK cell comprises one or more transgenes, wherein the loading comprises exposure of engineered NK cells to the one or more antibody for an amount of time effective for binding of the antibody to the engineered NK cell surface, and wherein the one or more transgenes comprise a CD70 targeting CAR comprising a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5, and/or a TROP2 targeting CAR comprising a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9. In some embodiments, loading the engineered NK cells comprises exposure of the engineered NK cells to the one or more antibody for greater than or equal to about 1 hour, or less than or equal to about 1 hour. In some embodiments, engineered NK cells remain loaded with antibody for at least about or about 1 hour, 4 hours, 8 hours, 12 hours, 1 day, 2 days, 3 days, 4 days, 5 days, 6 days, 7 days, 8 days, 9 days, 10 days, 11 days, or 12 days. In some embodiments, the one or more antibody is bound to the engineered NK cell through interaction with a CD16 surface protein. In some embodiments, the engineered NK cells are derived from cord blood. In some embodiments, the engineered NK cells are activated ex-vivo. In some embodiments, the engineered NK cells are pre-activated with IL-18, IL-15, and IL-12. In some embodiments, the engineered NK cells comprise one or more additional transgenes, optionally comprising an engineered receptor. In some embodiments, the engineered receptor comprises a chimeric antigen receptor (CAR), a T cell receptor, chemokine receptor, chimeric cytokine receptor, or any combination thereof. In some embodiments, the engineered receptor comprises a CAR or a TCR. In some embodiments, the engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof. In some embodiments, the engineered NK cell comprises a construct at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4. In some embodiments, the engineered NK cell comprises a construct at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10. In some embodiments, the one or more transgenes comprise a cytokine. In some embodiments, the one or more heterologous cytokine comprises IL-2, IL-4, IL-7, IL-12, IL-15, IL-18, IL-21 and/or IL-23. In some embodiments, the cytokine comprises IL-2, IL-12, IL-15, IL-18, and/or IL-21. In some embodiments, the cytokine comprises, consists of, or consists essentially of IL-15 encoded by a sequence comprising, consisting of, or consisting essentially of at least or about, and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12. In some embodiments, the cytokine comprises, consists of, or consists essentially of IL-21 encoded by a sequence comprising, consisting of, or consisting essentially of at least or about, and/or comprising, consisting of, or consisting essentially of a sequence at least or about 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16. In some embodiments, the engineered NK cells comprise on or more engineered mutations in an endogenous gene. In some embodiments, one or more engineered mutation in an endogenous gene comprises a CISH, CD38, Glucocorticoid Receptor, and/or TGFBR2 gene mutation.

In particular embodiments, the efficacy of the compositions comprising complexes of the engineered NK cells and the antibodies is enhanced compared to their separate use. In some embodiments, the efficacy of the engineered NK cells and the antibodies is synergistic when used as a complex. In some embodiments, the efficacy of the engineered NK cells and the antibodies is additive.

In any method, the composition (and/or the at least a second therapeutic agent) may be administered intravenously, intraperitoneally, intratracheally, intratumorally, intramuscularly, endoscopically, intralesionally, percutaneously, subcutaneously, regionally, or by direct injection or perfusion or infusion.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description. It is to be expressly understood, however, that the disclosure is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1. Schematic diagram detailing one embodiment of an experimental plan for expanding cord blood derived NK cells and preloading them with antibody.

FIG. 2. Margetuximab bound to preactivated and expanded (PE) NK cells with higher affinity compared to normal expanded (NE) NK cells.

FIG. 3. Margetuximab-loaded NK cells showed enhanced cytotoxicity (Y axis) against HER2+ (e.g., SKOV3) tumor cells in a long-term Xcelligence killing assay (X axis tracks time in hours). NK cells were either normally expanded (NE) or preactivated and expanded (P+E).

FIGS. 4A-4C. Margetuximab-loaded NK cells led to enhanced tumor control in an NSG mouse model of HER2+ovarian cancer (e.g., SKOV3).

FIG. 5. Amivantamab bound to preactivated and expanded (PE) NK cells with higher affinity compared to normal expanded (NE) NK cells.

FIG. 6. Amivantamab-loaded NK cells showed enhanced cytotoxicity against EGFR+/cMET+ tumor cells (e.g., SKOV3, UMRC3, and PATC148) in a short term 51Cr release killing assay (X axis tracks effector to target (E:T) ratios of 20:1, 10:1, 5:1, or 1:1). NK cells were either normally expanded (NE) or preactivated and expanded (PE).

FIGS. 7A-7B. Amivantamab-loaded NK cells showed enhanced cytotoxicity against EGFR+/cMET+ tumor cells (e.g., SKOV3 and PATC148) in a long-term Xcelligence killing assay (X axis tracks time in hours). NK cells were either normally expanded (NE) or preactivated and expanded (PE).

FIGS. 8A-8C. Amivantamab-loaded NK cells led to enhanced tumor control in an NSG mouse model of EGFR+/c-MET+ovarian cancer (e.g., SKOV3).

FIG. 9. Imgatuzumab bound to preactivated and expanded (PE) NK cells with higher affinity compared to normal expanded (NE) NK cells.

FIG. 10. Imgatuzumab-loaded NK cells showed enhanced cytotoxicity against EGFR+tumor cells (e.g., SKOV3, UMRC3, and PATC148) in a short term 51Cr release killing assay. NK cells were either normally expanded (NE) or preactivated and expanded (P+E).

FIGS. 11A-11C. Imgatuzumab-loaded NK cells showed enhanced cytotoxicity (Y axis, normalized cell index) against EGFR+ tumor cells (e.g., SKOV3, PATC148, and UMRC3) in a long-term Xcelligence killing assay (X axis tracks time in hours). NK cells were either normally expanded (NE) or preactivated and expanded (P+E).

FIGS. 12A-12C. Imgatuzumab-loaded NK cells led to enhanced tumor control in an NSG mouse model of EGFR+ovarian cancer (e.g., SKOV3).

FIG. 13. Is a series of western blots showing that EGFR was expressed on various tumor cell lines, including pancreatic (e.g., Capan-1, Capan-2, MIA-Capa2, CFPAC-1, PANC-1, BcPC3), colorectal (e.g., WiDr, HCT15, RKO, LOVO, SW403, LS174T, HCT116, SW116), kidney (e.g., 786-0, UMRC3, A498, 769-P, ACHN, Caki-1, Caki-2, A704), glioblastoma multiforme (GBM) (e.g., GSC272, GSC8-11, GSC20, and GSC267), breast (e.g., MDA-231, BCX010), and renal cancer (e.g., SN12C) tumor cell lines. B-actin was used as a loading control.

FIG. 14. Imgatuzumab bound to NK cells and EGFR+ tumor cell lines when added to cells at 1 μg or 10 μg, analyzed by flow cytometry.

FIG. 15. Imgatuzumab bound to non-transduced (NT) or transduced (e.g., with a chimeric antigen receptor (CAR)) NK cells. Further, Imgatuzumab stayed bound to NT or CAR NK cells (e.g., engineered NK cells) and was retained on their cell surface for at least 5 days.

FIG. 16. Imgatuzumab-loaded NK cells displayed enhanced killing of breast (e.g., MDA-231 cell line), pancreatic (e.g., pancreatic ductal adenocarcinoma) (PDAC) (e.g., PATC-148 cell line), and colorectal (CRC) (e.g., WiDR cell line) cell lines in vitro when compared to non-loaded NK cells.

FIG. 17. Imgatuzumab loaded engineered NK cells (e.g., CAR NK+Imga 1 μg) displayed enhanced killing of UMRC3 (RCC) cells compared to Imga-treated tumor cells alone, non-loaded non-transduced (NT) (e.g., non-engineered) NK cells, Imgatuzumab loaded NT-NK cells (e.g., NT−NK+Imga 1 μg), or engineered non-loaded NK (CAR NK) cells in vitro.

FIG. 18. Imgatuzumab loaded engineered NK cells (e.g., CAR NK+Imga 1 μg) showed enhanced killing of SKOV3 (ovarian Cancer) cells compared to Imga-treated tumor cells alone, non-loaded non-transduced (NT) (e.g., non-engineered) NK cells, Imgatuzumab loaded NT-NK cells (e.g., NT−NK+Imga 1 μg), or engineered non-loaded NK (CAR NK) cells in vitro.

FIG. 19. Imgatuzumab loaded engineered NK cells (e.g., CAR NK+Imga 1 μg) showed enhanced killing of PATC-148 (PDAC) cells compared to Imga-treated tumor cells alone, non-loaded non-transduced (NT) (e.g., non-engineered) NK cells, Imgatuzumab loaded NT-NK cells (e.g., NT−NK+Imga 1 μg), or engineered non-loaded NK (CAR NK) cells in vitro.

FIG. 20. Antibodies (e.g., Imgatuzumab) were retained on the surface of NK cells for at least 12 days following antibody loading. NK cells were left unloaded (−Imga) or loaded with Imgatuzumab (100 μg/ml final concentration) (+Imga) for one hour at 37° C. in Click's/RPMI media and were washed twice with PBS (5 minutes for each wash) before validating the binding of Imgatuzumab with flow cytometry. NK cells were stained with Alexa-Fluor647 affinity-purified F(ab′)₂ fragment goat anti-human IgG (H+L) antibody at different time points (1 hour, 3 days, 5 days, 7 days, and 12 days) and analyzed by flow cytometry.

FIGS. 21A-21B. Imgatuzumab alone did not cause cytotoxicity against ovarian cancer or PDAC cell lines. Xcelligence assays were performed to assess the cytotoxicity of Imgatuzumab against ovarian cell lines (e.g., SKOV3, FIG. 21A) and pancreatic ductal adenocarcinoma (PDAC) cell lines (e.g., PATC148, FIG. 21B). Graphs showed normalized cell index (Y axis) plotted over time (X axis, hours). Cell lines cultured alone served as negative controls, and cell lines lysed with SDS served as positive control. Statistical analysis comprised two-way ANOVA.

FIGS. 22A-22B. Imgatuzumab loaded NK cells showed enhanced killing of ovarian cancer (e.g., SKOV3) cell lines and PDAC (e.g., PATC148) cells when compared to non-loaded NK cells in long-term Xcelligence cytotoxicity assays. NK cells were loaded with Imgatuzumab (100 μg Imgatuzumab, e.g., 100 μg/ml final concentration Imgatuzumab in 1 ml of NK cells) for one hour at 37° C. in Click's/RPMI media and then washed twice with PBS (5 minutes for each wash) before co-culturing with tumor cells at 1:1 effector to target ratios. When compared to non-loaded NK cells, Imgatuzumab loaded NK cells showed increased cytotoxic activity against SKOV3 (FIG. 22A) and PATC148 (FIG. 22B) cells. Cell lines cultured alone served as negative control and cell line lysed with SDS served as positive control, statistical analysis comprised two-way ANOVA.

FIG. 23. Imgatuzumab loaded NK cells showed enhanced killing of colorectal cancer (CRC) cells (e.g., WiDR) when compared to non-loaded NK cells in long-term Xcelligence cytotoxicity assays. NK cells were loaded with Imgatuzumab (100 μg/ml final concentration) for one hour at 37° C. in Click's/RPMI media and then washed twice with PBS (5 minutes for each wash) before co-culturing with tumor cells at 1:1 effector to target ratio. Statistical analysis comprised two-way ANOVA.

FIGS. 24A-24B. Imgatuzumab loaded NK cells showed enhanced killing of Lung cancer cells (e.g., CRL-5922 and HTB-58) when compared to non-loaded NK cells in Xcelligence long-term cytotoxicity assays. NK cells were loaded with Imgatuzumab (100 μg/ml final concentration) for one hour at 37° C. in Click's/RPMI media and then washed twice with PBS (5 minutes for each wash) before co-culturing with tumor cells at 1:1 effector to target ratios. Compared to non-loaded NK cells, Imgatuzumab loaded NK cells showed increased cytotoxic activity against CRL-5922 (FIG. 24A) and HTB-58 (FIG. 24B) lung cancer cell lines. Cell lines cultured alone served as negative control and cell line lysed with SDS served as positive control. Statistical analysis comprised two-way ANOVA.

FIGS. 25A-25B. Imgatuzumab loaded NK cells showed enhanced killing of Breast cancer cells (e.g., MDA 468 and SKBR3) when compared to non-loaded NK cells in Xcelligence long-term cytotoxicity assays. NK cells were loaded with Imgatuzumab (100 μg/ml final concentration) for one hour at 37° C. in Click's/RPMI media and then washed twice with PBS (5 minutes for each wash) before co-culturing with tumor cells at 1:1 effector to target ratios. Compared to non-loaded NK cells, Imgatuzumab loaded NK cells showed increased cytotoxic activity against CRL-5922 (FIG. 25A) and HTB-58 (FIG. 25B) lung cancer cell lines. Cell lines cultured alone served as negative control and cell line lysed with SDS served as positive control. Statistical analysis comprised two-way ANOVA.

FIGS. 26A-26B. Imgatuzumab loaded NK cells showed enhanced killing of PDAC (e.g., PATC148) tumor spheroids when compared to non-loaded NK cells in Incucyte live cell imaging assays. FIG. 26A shows representative images of PATC148 spheroids (20,000 tumor cells transduced with GFP and allowed to form spheroids for 48 hours) left alone or treated with either 50,000 non-loaded NK cells or 50,000 NK cells preloaded with Imgatuzumab (100 μg/ml final concentration) at different time points. The data showed significant decreases in spheroid size in wells treated with Imgatuzumab loaded NK cells. FIG. 26B quantification of total integrated green intensity over time. Statistical analysis comprised two-way ANOVA.

FIGS. 27A-27B. Imgatuzumab loaded NK cells showed enhanced cytotoxicity against CRC (e.g., WiDR) tumor spheroids when compared to non-loaded NK cells in an Incucyte live cell imaging assay. FIG. 27A shows representative images of WiDR spheroids (20,000 tumor cells transduced with GFP and allowed to form spheroids for 48 hours) left alone or treated with either 50,000 non-loaded NK cells or 50,000 NK cells preloaded with Imgatuzumab (100 μg/ml final concentration) at different time points. The data showed significant decreases in spheroid size in wells treated with Imgatuzumab loaded NK cells. FIG. 27B quantification of total integrated green intensity over time. Statistical analysis comprised two-way ANOVA.

FIGS. 28A-28B. Imgatuzumab loaded NK cells showed enhanced cytotoxicity against UMRC3 (RCC) tumor spheroids compared to non-loaded NK cells in an Incucyte live cell imaging assay. FIG. 28A shows a representative images of UMRC3 spheroids (20,000 tumor cells transduced with GFP and allowed to form spheroids for 48 hours) left alone or treated with either 50,000 non-loaded NK cells or 50,000 NK cells preloaded with Imgatuzumab (100 μg/ml final concentration) at different time points. The data showed significant decreases in spheroid size in wells treated with Imgatuzumab loaded NK cells. FIG. 28B quantification of total integrated green intensity over time. Statistical analysis comprised two-way ANOVA.

FIGS. 29A-29B. Imgatuzumab loaded NK cells showed enhanced cytotoxicity against GBM (e.g., GSC20) tumor spheroids compared to non-loaded NK cells in an Incucyte live cell imaging assay. FIG. 29A shows representative images of GSC20 spheroids (20,000 tumor cells transduced with mCherry and allowed to form spheroids for 48 hours) left alone or treated with either 50,000 non-loaded NK cells or 50,000 NK cells preloaded with Imgatuzumab (100 μg/ml final concentration) at different time points. The data showed significant decreases in spheroid size in wells treated with Imgatuzumab loaded NK cells when compared to non-loaded NK cells.

FIG. 29B quantification of total integrated red intensity over time. Statistical analysis comprised two-way ANOVA.

FIGS. 30A-30D. Imgatuzumab loaded NK cells led to enhanced tumor control in an NSG mouse model of EGFR+PDAC (e.g., PATC148). FIG. 30A schema of experimental plan for PATC148 mouse model. On day-7 mice 10 week old female NSG mice were injected intravenously (IV) with 0.3×10⁶ PATC148 cells transduced with Fireflyluciferase (FFluc), on day-2 BLI was performed to document engraftment, on day-1 mice were irradiated with 300cGy, and on day zero, 10⁷ fresh NK cells alone or 10⁷ fresh NK cells pre-loaded with Imgatuzumab (100 μg/ml final concentration), washed twice with saline (5 minutes each wash) and then suspended in saline were injected IV into each mouse. Test mice were then injected with 5 mg/kg Imgatuzumab weekly for three weeks. FIG. 30B bioluminescent imaging showing tumor growth over time in mice engrafted with PATC148 transduced with Fireflyluciferase (Ffluc) and either left untreated or treated with NK cells alone, or NK cells pre-loaded with Imgatuzumab. FIG. 30C graph showing average radiance over time of the 3 groups of mice described in FIG. 30C. FIG. 30D Survival curves of the 3 groups of mice described in FIG. 30B. Statistical analysis comprised two-way ANOVA.

FIG. 31. Imgatuzumab loaded NT NK cells (as shown in FIG. 20) and TROP2 CAR-NK cells retained antibody on their cell surface for at least 12 days. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI) and either left unmodified non-transduced (NT) or transduced with TROP2 CAR and IL-15 (TROP2) on day 6. NT NK cells or TROP2 CAR NK cells were loaded with Imgatuzumab (100 μg/ml final concentration) for one hour at 37° C. in Click's/RPMI media and were washed twice with PBS (5 minutes for each wash) before validating the binding of Imgatuzumab with flow cytometry. NK cells were stained with Alexa-Fluor647 affinity-purified F(ab′)₂ fragment goat anti-human IgG (H+L) antibody at different time points (1 hour, 3 days, 5 days, 7 days, and 12 days) and analyzed by flow cytometry. The results showed that Imgatuzumab was retained on surface of NT NK cells (FIG. 20) and TROP2 CAR NK cells (FIG. 31) for at least 12 days.

FIGS. 32A-32B. Imgatuzumab loaded TROP2 CAR-NK cells showed enhanced killing of ovarian cancer (e.g., SKOV3) cells and gastrointestinal stromal tumor (GIST) (e.g., OVCAR5) cells when compared to non-loaded NK cells in Xcelligence long-term cytotoxicity assays. NK cells either non-transduced (NT) or transduced with TROP2 CAR and IL-15 (TROP2/IL15) were loaded with Imgatuzumab (100 μg/ml final concentration) for one hour at 37° C. in click media and then washed twice with PBS (5 minutes for each wash) before co-culturing with tumor cells at 1:1 effector to target ratio. Compared to non-loaded NK cells, Imgatuzumab loaded NK cells showed increased cytotoxic activity against the target cells. Statistical analysis comprised two-way ANOVA.

FIG. 33. Imgatuzumab loaded NK cells maintained antibody on their surface after being frozen and thawed. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI) and either left unmodified non-transduced (NT) or transduced with TROP2 CAR and IL-15 (TROP2CAR/IL-15) on day 6. NT NK cells or TROP2CAR/IL-15 cells were loaded with Imgatuzumab (100 μg/ml final concentration) (+Imga) for one hour at 37° C. in Click's/RPMI media, were washed twice with PBS (5 minutes for each wash), and were then deactivated with Dasatinib prior to cryopreservation. NK cells were then thawed and stained with Alexa-Fluor647 affinity-purified F(ab′)₂ fragment goat anti-human IgG (H+L) antibody and analyzed by flow cytometry.

FIGS. 34A-34D. Imgatuzumab loaded NT and TROP2 CAR-NK cells showed enhanced cytotoxicity against multiple cancer cell lines when compared to non-loaded NK cells after freeze/thaw. NK cells either non-transduced (NT) or transduced with chimeric antigen receptor TROP2 CAR and IL-15 (TROP2/IL15) were loaded with Imgatuzumab (100 μg/ml final concentration) for one hour at 37° C. in Click's/RPMI media, were then washed twice with PBS (5 minutes for each wash), and were then deactivated with Dasatinib prior to cryopreservation. Cells were then thawed and used in Xcelligence cytotoxicity assays against multiple cancer cell lines (e.g., PDAC tumor cell line PATC148 (FIG. 34A), CRC cell line WiDR (FIG. 34B), GIST cell line OVCAR5 (FIG. 34C), and ovarian cancer cell line SKOV3 (FIG. 34D)). Statistical analysis comprised two-way ANOVA.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

It is contemplated that any embodiment discussed in this specification can be implemented with respect to any method or composition of the invention, and vice versa. Furthermore, compositions of the invention can be used to achieve methods of the invention.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. In specific embodiments, an engineered cell is a cell that comprises one or more exogenous antigen receptors (e.g., chimeric antigen receptors, T cell receptors, etc.), suicide genes, cytokines and/or cytokine receptors (e.g., IL-15, IL-15R, etc.), chemokine/homing receptors, and/or modification of one or more endogenous genes.

The term “load,” “loaded,” or loading” as used herein refers to adoptive cell therapy cells having one or more antibodies bound to the cells on the surface of the cells.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “pre-activated” or “pre-activation” as used herein refers to exposure of NK cells to IL-12, IL-15 or IL-2, and/or IL-18 that results in increased signaling pathways related to NK cell effector function, such as enrichment of genes involved in the IFN-γ response, TNF signaling, IL-2/STAT5 signaling, IL-6/JAK/STAT3 signaling, mTOR pathway and/or genes related to inflammatory immune responses. In specific cases there is increased expression of TRAIL, NKp44 and/or CD69.

The term “pre-load,” “pre-loaded,” or “pre-loading” as used herein refers to adoptive cell therapy cells that have had one or more antibodies bound to the cells on the surface of the cells prior to use of the cells for any reason.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “subject,” as used herein, generally refers to an individual having a that has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasia, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical composition” via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof.

### I. Natural Killer (NK) Cells and Methods of Production

NK cells are emerging as an exciting source of cellular immunotherapy for patients with malignant hematologic disease as well as solid tumors; however, most studies using adoptively transferred NK cells have been limited by inadequate persistence, poor in vivo expansion and disappointing anti-tumor activity of the infused cells. Thus, a barrier to overcome in the field of NK immunotherapy is the need for biology-driven approaches to increase NK cell antitumor functionality, such as by manipulation of the cells prior to administration as therapy. Accordingly, in certain embodiments, the present disclosure provides methods for production of NK cells having enhanced efficacy of any kind compared to NK cells that are not so manipulated. Some embodiments of the present disclosure concern the isolation, activation, and expansion of NK cells, including for cancer immunotherapy.

In particular embodiments, the disclosure encompasses loaded, pre-activated, (optionally) and expanded (optionally) NK cells produced by the present methods that exhibit enhanced anti-tumor functionality against cancer. Pre-activated and expanded NK cells also display enhanced antibody-dependent cellular cytotoxicity (ADCC), in specific embodiments.

In particular embodiments, the disclosure encompasses engineered, loaded, pre-activated, (optionally) and expanded (optionally) NK cells produced by the present methods that exhibit enhanced anti-tumor functionality against cancer. In specific embodiments, loaded and/or engineered NK cells display enhanced antibody-dependent cellular cytotoxicity (ADCC) relative to non-loaded and/or non-engineered NK cells. In particular embodiments, loaded and/or engineered NK cells display enhanced long-term cytotoxicity relative to non-loaded and/or non-engineered NK cells. In particular embodiments, loaded and/or engineered NK cells display enhanced long-term cytotoxicity against solid tumors and/or tumor spheroids relative to non-loaded and/or non-engineered NK cells. In particular embodiments, loaded and/or engineered NK cells display enhanced synergistic long-term cytotoxicity relative to non-loaded and/or non-engineered NK cells. In particular embodiments, loaded and/or engineered NK cells display enhanced synergistic long-term cytotoxicity against solid tumors and/or tumor spheroids relative to non-loaded and/or non-engineered NK cells. In particular embodiments, Imgatuzumab loaded and engineered NK cells display enhanced synergistic long-term cytotoxicity against solid tumors and/or tumor spheroids relative to non-loaded and/or non-engineered NK cells.

### A. NK Cells, Generally

In certain embodiments, NK cells utilized in the methods (e.g., NK cells that will be engineered) may be derived from any suitable source, such as cord blood (CB), including human CB. In particular cases, the NK cells are not derived from cord tissue (the insulating material (i.e., the Wharton's jelly) surrounding the vessels of the umbilical cord). In alternative embodiments, the NK cells are derived from human peripheral blood mononuclear cells (PBMC), unstimulated leukapheresis products (PBSC), human embryonic stem cells (hESCs), induced pluripotent stem cells (iPSCs), and/or bone marrow, or NK cells lines derived from patients, such as NK-92, by methods well known in the art. In particular embodiments, the NK cells are isolated from pooled CB. The CB may be pooled from 2, 3, 4, 5, 6, 7, 8, 9, 10, or more units. The NK cells may be autologous or allogeneic with respect to a recipient individual. The isolated NK cells may or may not be haplotype matched for the subject to be administered the cell therapy. NK cells may or may not be detected by specific surface markers, such as CD16 and/or CD56 in humans. In some cases, the NK cells are depleted for the presence of one or more surface markers, such as depleted for CD3+, CD14+ and/or CD19+ cells. In specific embodiments, the NK cells are CD3−CD56+.

In certain aspects, the NK cells are isolated by the previously described method of ex vivo expansion of NK cells (Spanholtz et al., 2011; Shah et al., 2013). In this method, CB mononuclear cells are isolated by ficoll density gradient centrifugation. The cell culture may be depleted of any cells expressing CD3 and may be characterized to determine the percentage of CD56⁺/CD3⁻ cells or NK cells. In other methods, umbilical CB is used to derive NK cells by the isolation of CD34⁺ cells.

### B. Loading of NK Cells

In particular embodiments, the NK cells are loaded with antibodies prior to use. The NK cells may be loaded in any specific manner, including in culture (e.g., incubation) or immediately before infusion, for example, to produce a complex of NK cells with the antibodies. The culture (e.g., incubation) conditions are suitable enough to allow for an effective amount of antibody to bind to the surface of the NK cells. In the case of use of monospecific antibodies, the Fc region of the monospecific antibody binds the NK cell while the antigen binding domain of the monospecific antibody is free to bind its target antigen. In certain embodiments of cases of use of multispecific antibodies, one or more antigen binding domains of the antibody can bind the surface of the NK cells, such as through an antigen on the surface of the NK cells (for example but not limited to, NKp30, NKp44, NKp46, CD16, CD32, CD64, KIRs, and the like), and the other antigen binding domain is free to bind its target antigen. In certain embodiments of cases of use of multispecific antibodies, one or more antigen binding domains of the antibody can bind one or more target antigens.

The culture conditions by which the NK cells become loaded may or may not be of a particular type having one or more specific parameters. In particular embodiments, the loading of the NK cells occurs in culture at a specific temperature, such as 37° C., although in alternative embodiments the temperature is 36° C. or 38° C., or lower or higher. The duration of the loading step may be for any suitable amount of time, such as in a range of one minute to 24 hours or longer. For example, the range may be in the range of 1 min to 24 hrs, 1 min to 18 hrs, 1 min to 12 hours, 1 min to 6 hrs, 1 min to 1 hr, 30 min to 24 hrs, 30 min to 18 hrs, 30 min to 12 hrs, 30 min to 6 hrs, 30 min to 1 hr, 1-24 hrs, 1-18 hrs, 1-12 hrs, 1-6 hrs, 6-24 hrs, 6-18 hrs, 6-12 hrs, 12-24 hrs, 12-18 hrs, or 18-24 hrs. In some embodiments, the duration of the loading step may be greater than or equal to approximately 6, 8, 10, 12, 14, 16, 18, 20, 22, 24, 26, 28, 30, 32, 34, 36, 38, 40, 42, 44, 46, or 48 hours, or any range derivable therein. In specific embodiments, the cell culture media is basal media or complex media. In some cases, the culture comprises one or more reagents that were utilized during pre-activation and/or expansion steps, while in other cases the culture does not. In specific embodiments, the culture comprises one or more cytokines, including one or more of IL-12, IL-15, IL-2, and IL-18, for example. In some embodiments, the culture comprises APCs of any kind.

In some embodiments, loading of the NK cells is conducted such that antibodies remain bound to the NK cell surface following washing of the NK cells. In some embodiments, NK cells are loaded with antibodies under conditions that are suitable for retention of at least a detectable fraction of the antibody on the NK cell surface for at least or exactly 12 hours, 24 hours, 36 hours, 48 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, 252 hours, 264 hours, 276 hours, 288 hours, 300 hours, 312 hours, or greater than 312 hours, or any range derivable therein. In some embodiments, NK cells are loaded with antibodies under conditions that are suitable for retention of the antibody on the NK cell surface for at least 1, 2, 3, 4, 5, 6, 7, 8, 9, 10, 11, 12, 13, 14, or 15, or longer than 15 days, or any range derivable therein. In some embodiments, NK cells are loaded with antibodies under conditions that are suitable for retention of the antibody on the NK cell surface following cryopreservation and/or thawing. In some embodiments, confirmation of loading of an antibody to an NK cell surface can be determined using flow cytometry.

In some embodiments, NK cells are loaded with at least, exactly, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/ml final concentration, or greater than 100 μg/ml final concentration, or any range derivable therein, of antibody. In some embodiments, NK cells are loaded with at least, exactly, or about 1, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, or 1000 μg/ml final concentration, or greater than 1000 μg/ml final concentration, or any range derivable therein, of antibody.

In some embodiments, NK cells and antibodies are incubated such that at least or exactly about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, of NK cells are complexed with an antibody.

In some embodiments, NK cells and antibodies are incubated such that at least or exactly about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, of the antibody remains bound to the NK cells for at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, or 12 days, or any range derivable therein, following incubation of the NK cells and antibody.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 3 days after incubation and washing. In certain embodiments, relative to the amount of antibodies complexed to engineered NK cells measured about 1 hour after incubation and washing, at least about 9%, of the antibodies are bound to the NK cell surface at least 3 days after incubation and washing.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 5 days after incubation and washing. In certain embodiments, relative to the amount of antibodies complexed to engineered NK cells measured about 1 hour after incubation and washing, at least about 6% of the antibodies are bound to the NK cell surface at least 5 days after incubation and washing.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 7 days after incubation and washing. In certain embodiments, relative to the amount of antibodies complexed to the engineered NK cells measured about 1 hour after incubation and washing, at least about 4% of the antibodies are bound to the NK cell surface at least 7 days after incubation and washing.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 12 days after incubation and washing. In certain embodiments, relative to the amount of antibodies complexed to the engineered NK cells measured about 1 hour after incubation and washing, at least about 3% of the antibodies are bound to the NK cell surface at least 12 days after incubation and washing.

In certain embodiments, a composition comprising engineered NK cells and antibodies is cryopreserved. In certain embodiments, a composition comprising engineered NK cells and antibodies is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

In certain embodiments, relative to the amount of antibodies complexed to non-cryopreserved engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, or greater than 90%, or any range derivable therein, of the antibodies are bound to the NK cell surface after thawing from cryopreservation. In certain embodiments, relative to the amount of antibodies complexed to non-cryopreserved engineered NK cells measured about 1 hour after incubation and washing, at least about 30% of the antibodies are bound to the NK cell surface after thawing from cryopreservation. In certain embodiments, relative to the amount of antibodies complexed to non-cryopreserved engineered NK cells measured about 1 hour after incubation and washing, at least about 40% of the antibodies are bound to the NK cell surface after thawing from cryopreservation. In certain embodiments, relative to the amount of antibodies complexed to non-cryopreserved engineered NK cells measured about 1 hour after incubation and washing, at least about 50% of the antibodies are bound to the NK cell surface after thawing from cryopreservation. In certain embodiments, relative to the amount of antibodies complexed to non-cryopreserved engineered NK cells measured about 1 hour after incubation and washing, at least about 60% of the antibodies are bound to the NK cell surface after thawing from cryopreservation.

In some embodiments, incubation of the NK cells and antibody occurs in any suitable NK cell media known to one of skill in the art. In certain embodiments, incubation of the NK cells and antibody is performed in media comprising, consisting of, or consisting essentially of Click's/RPMI media. In certain embodiments, incubation of the NK cells and antibody is performed in media comprising, consisting of, or consisting essentially of Click's media. In certain embodiments, incubation of the NK cells and antibody is performed in media comprising, consisting of, or consisting essentially of RPMI media. In certain embodiments, incubation of the NK cells and antibody is performed in media comprising, consisting of, or consisting essentially of SCGM media.

The antibodies of the compositions are subjected in an effective amount to an effective amount of NK cells of the disclosure, thereby producing a complex that is “chimeric antigen receptor-like.” In particular, an antigen binding domain of the antibody binds to the NK cells, such as through the antigen that is a cell surface protein. A plurality of antibodies may be subjected to a plurality of NK cells such that there are multiple complexes of cell/antibody. The antibodies may be of any time, including monospecific, bispecific, or multispecific, and in specific cases the antibody engages both the NK cell and a target antigen through an antigen binding domain of the antibody (such as with engagers in the art that are fusion proteins consisting of two single-chain variable fragments (scFvs) of different antibodies). In examples wherein the antibody is monospecific, an antigen binding domain of the antibody binds a target antigen, such as a cancer antigen, and another part of the antibody binds the NK cells, such as an Fc region of the antibody. In cases wherein the antibody is multispecific, one or more antigen binding domains of the antibody binds the NK cell (such as through an NK cell surface antigen) and one or more antigen binding domains of the antibody binds one or more target antigens. The multispecific antibody may be bispecific, trispecific, or tetraspecific, for example. In cases wherein the antibody is trispecific or tetraspecific, the additional antigen binding domains may bind other cells, such as stem cells.

In particular embodiments, the antibodies may bind any NK cell surface antigen (that may or may not be receptors) on NK cells, such as CD16 (including CD16a or CD16b), CD32, CD56, CD64, a c-type lectin such as NKG2D, NKG2C, a costimulatory molecule such as CS1, DNAM, 2B4, CD2, an NCR, NKp30, NKp44, NKp46, or KIR, and redirect the NK cells to a target, thus increasing the response and specificity against different tumors.

In some embodiments, the antibodies may bind any suitable antigen (e.g., antigens described herein, such as those that are described as targets of CARs and/or TCRs). In particular embodiments, an antibody targets CD123. In particular embodiments, an antibody targets EGFR. In particular embodiments, an antibody targets EGFR2. In particular embodiments, an antibody is bi-specific and targets EGFR and c-MET. In particular embodiments, an antibody is Imgatuzumab, Amivantamab, and/or Margetuximab.

Generation of the complexes may be by any suitable means, such that the conditions are sufficient for the appropriate region of the antibody to bind the appropriate surface region of the NK cell. Any particular medium may be utilized, in certain instances. In specific cases, Plasma-Lyte A and/or human serum albumin are utilized, wherein in other cases they are not. Once the complexes are formed in culture, they may or may not be washed prior to administration to the subject, such as through infusion. In alternative embodiments, the NK cells and the antibodies are administered separately, and the complexes form in vivo. In certain embodiments, a composition comprising NK cells and antibody is washed with PBS to remove unbound antibody. In certain embodiments, a composition comprising NK cells and antibody is washed at least 1, 2, 3, 4, or 5, or greater than 5 times to remove unbound antibody. In certain embodiments, a composition comprising NK cells and antibody is was for at least 1, 2, 3, 4, 5, 6, 7, 8, 9, or 10, or greater than 10 minutes to remove unbound antibody. In certain embodiments, a composition comprising NK cells and antibody is washed twice to remove unbound antibody. In certain embodiments, a wash is for exactly, or about 5 minutes. In certain embodiments, a wash comprises agitation of the composition, e.g., using a cell shaker.

### C. Pre-Activation

In some embodiments, the NK cells are pre-activated prior to administration to a recipient individual. The pre-activation step may or may not occur before any expansion step. In specific embodiments, the NK cells are pre-activated with one or more cytokines, and in specific embodiments, the NK cells are pre-activated with one or more of IL-12, IL-15, IL-2, and IL-18 and including two, three, or more. In cases wherein less than all three of IL-12, IL-15, IL-2, and IL-18 are utilized, it may be that IL-12 and IL-15 but not IL-18; or IL-12 and IL-18 but not IL-15; or IL-15 and IL-18 but not IL-12. IL-2 may or may not be substituted for IL-15.

In particular embodiments, the pre-activation cytokines may be IL-12, IL-15, and IL-18. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, and specifically about 16 hours in some cases. The pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/ml, specifically about 50 ng/mL. In some cases, the pre-activation culture comprises IL-12 at a concentration of 0.1-150 ng/ml, including at a concentration of 1-20 ng/ml, such as a concentration of 10 ng/mL. In alternative embodiments the NK cells may be stimulated with IL-2, or other cytokines that bind the common gamma-chain (e.g., IL-7, IL-21, and others), and this may be in addition to IL-12, IL-15, and IL-18 or as an alternative to one or more of them. In such cases, the pre-activation culture may comprise IL-12 at a concentration of 0.1-150 ng/mL, such as 0.5-50 ng/mL, particularly 1-20 ng/mL, such as 2, 3, 4, 5, 6, 7, 8, 9, 10, 11, 12, 13, 14, or 15 ng/ml, specifically about 10 ng/mL.

### D. Expansion

In particular embodiments, NK cells are expanded to increase their quantity prior to administration to an individual in need thereof. The expanded cells may or may not be derived from pre-activated NK cells such that a pre-activation step may occur before an expansion step. The NK cell expansion step may be of any suitable such that the NK cell population is expanded, but in specific cases the expansion step utilizes particular one or more reagents, such as in culture, to enhance their expansion. In certain cases the NK cells may not be expanded. IL-2 or IL-15 or IL-18 or any combination of the cytokines may be added to the expansion culture before or during expansion. The NK cells can be expanded ex vivo in flasks or in one of several different bioreactor configurations with continuous perfusion of media/additives, in specific embodiments.

In specific cases, the NK cells (whether pre-activated or not) may be washed (e.g., with PBS or Plasma Lyte or human serum albumin or culture media or combinations thereof) prior to and/or after expansion, such as 2, 3, 4, or 5 times, specifically 3 times. In particular embodiments, the NK cells are expanded in the presence of artificial antigen presenting cells (aAPCs). In particular embodiments, the NK cells are expanded in the presence of fragments of aAPCs. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58) or CD48. In particular, the aAPCs may be K562 cells, such as K562 cells engineered to express CD137 ligand and mIL-21. The engineering may be by any method known in the art, such as retroviral transduction, although any viral or non-viral vector may be utilized. The aAPCs may or may not be irradiated. The expansion may be for a particular duration in time, such as for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise one or more cytokines to promote expansion, such as IL-2. The IL-2 may be present at a concentration of about 10-500 U/mL, such as 100-300 U/mL, particularly about 200 U/mL. The IL-2 may be replenished in the expansion culture, including at a certain frequency, such as every 2-3 days. The aAPCs may be added to the culture at least a second time, such as at about 7 days of expansion. Any cytokine(s) used in the pre-activation and/or expansion steps may be recombinant human cytokines.

Following expansion, the NK cells may be immediately utilized in any manner, such as complexed with one or more antibodies, or they may be stored, such as by cryopreservation. In certain aspects, the cells may be propagated for days, weeks, or months ex vivo as a bulk population within about 1, 2, 3, 4, or 5 days.

Activated and/or expanded NK cells can secrete type I cytokines, such as interferon-Y, tumor necrosis factor-a and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

Thus, with respect to particular pre-activation and expansion aspects of the disclosure, in specific embodiments the NK cells pre-activated with any combination of IL-12, IL15, and/or IL-18 followed by expansion with aAPCs, such as K562 cells expressing mIL-21 and CD137 ligand, provide a highly potent cellular product. Thus, methods are provided using the present NK cells for the treatment of various diseases, such as immunotherapy of patients with cancer. In an exemplary method, the isolated NK cells may be subjected to a brief period, such as about 16 hours, of pre-activation with a combination of cytokines, such as interleukin-12 (IL-12), IL-15, and/or IL-18, followed by expansion using artificial antigen presenting cells (aAPCs), such as K562 feeder cells expressing membrane-bound IL-21 and CD137 ligand, and/or exogenous IL-2. IL-2 or IL-15 or IL-18 or any combination of the cytokines may be added to the expansion culture at least a second time.

### E. Engineering of NK Cells

As described herein, in some embodiments, the NK cells are modified (e.g., engineered) compared to natural NK cells in one or more ways. The engineered NK cells may be manipulated by the hand of man to be engineered to express one or more transgenes. The engineered NK cells may be manipulated by the hand of man to be engineered to express one or more heterologous proteins. Alternatively, or in addition, the engineered NK cells may be modified to have reduced or inhibited expression of one or more endogenous genes. Alternatively, or in addition, the engineered NK cells may be modified to express one or more chemokines, chemokine receptors, cytokines, cytokine receptors, and/or suicide genes. In certain embodiments, NK cells are engineered to express a CD16, CD32, CD64, or other FCR binding regions. In some embodiments, an engineered NK cell does not comprise a non-naturally occurring CD16 gene. In some embodiments, an engineered NK cell does not comprise a transgene encoding a CD16 gene. In certain embodiments, NK cells are engineered to express a CD16 gene. In certain embodiments, NK cells are engineered to express a wild type CD16 gene. In certain embodiments, NK cells are engineered to express a CD32 gene. In certain embodiments, NK cells are engineered to express a CD64 gene. In certain embodiments, NK cells are engineered to express a transgenic receptor that is a target of a multi-specific antibody.

1. Engineered Antigen Receptors

The NK cells may be genetically modified to express one or more engineered antigen receptors, including at least one or more chimeric antigen receptors (CARs) and/or one or more TCRs. In specific embodiments, the engineered antigen receptors are directed to target one or more cancer antigens, and the antigen may or may not be the same antigen to which the multispecific antibody (e.g., engager) is directed.

The NK cells may be modified to encode at least one CAR, and the CAR may be first generation, second generation, or third or a subsequent generation, for example. The CAR may or may not be bispecific for two or more different antigens. The CAR may comprise one or more costimulatory domains. NK cells may also be modified to express a receptor to enhance their binding to the antibody, such as CD16, CD32 and/or CD64 receptor. Each costimulatory domain may comprise the costimulatory domain of any one or more of, for example, members of the TNFR superfamily, CD28, CD137 (4-1BB), CD134 (OX40), DAP10, DAP12, CD27, CD2, CD5, ICAM-1, LFA-1 (CD11a/CD18), Lck, TNFR-I, TNFR-II, Fas, CD30, CD27, NKG2D, 2B4M, CD40 or combinations thereof, for example. In specific embodiments, the CAR comprises CD3zeta. In certain embodiments, the CAR lacks one or more specific costimulatory domains; for example, the CAR may lack 4-1BB and/or lack CD28.

In particular embodiments, the CAR polypeptide in the cells comprises an extracellular spacer domain that links the antigen binding domain and the transmembrane domain, and this may be referred to as a hinge. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha or CD4, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1 and in certain aspects the CAR polypeptide comprises a particular IgG1 hinge amino acid sequence or is encoded by a particular IgG1 hinge nucleic acid sequence.

The transmembrane domain in the CAR may be derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD30, CD33, CD37, CD64, CD80, CD86, CD 134, CD137, CD154, ICOS/CD278, GITR/CD357, NKG2D, and DAP molecules, such as DAP10 or DAP12. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain.

In certain embodiments, the CAR may be co-expressed with one or more cytokines to improve persistence when there is a low amount of tumor-associated antigen. For example, the CAR may be co-expressed with one or more cytokines, such as IL-7, IL-2, IL-15, IL-12, IL-23, IL-18, IL-21, IL-7, GMCSF, or a combination thereof. In some embodiments, the NK cells expressing the CAR are engineered to express one or more heterologous cytokines and/or are engineered to upregulate normal expression of one or more heterologous cytokines. The cells may or may not be transduced or transfected for one or more cytokines on the same vector as other genes.

In certain embodiments, a CD27 CAR targets CD70. In some embodiments, a CAR comprises a CD28 derived transmembrane domain. In some embodiments, a CAR comprises a CD3 zeta intracellular stimulatory domain. In some embodiments, a CAR is co-expressed with IL-15.

In certain embodiments, an NK cell is engineered to express a polynucleotide comprising a sequence with 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, sequence identity according to SEQ ID NOs: 1, 3, 4, 6, 8, 10, 11, 13, or 15. In certain embodiments, an NK cell is engineered to express a polynucleotide comprising a sequence encoding a polypeptide with 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, sequence identity according to SEQ ID NOs: 2, 5, 7, 9, 12, 14, or 16.

SEQ ID NO: 1 - Exemplary polynucleotide sequence
comprising a CD27 CAR construct targeting a CD70
antigen.
(SEQ ID NO: 1)
ATGGCACGGCCACATCCCTGGTGGCTGTGCGTTCTGGGGACCCTGGTGGGGCTCTCAGCTACTC
CAGCCCCCAAGAGCTGCCCAGAGAGGCACTACTGGGCTCAGGGAAAGCTGTGCTGCCAGATGTG
TGAGCCAGGAACATTCCTCGTGAAGGACTGTGACCAGCATAGAAAGGCTGCTCAGTGTGATCCT
TGCATACCGGGGGTCTCCTTCTCTCCTGACCACCACACCCGGCCCCACTGTGAGAGCTGTCGGC
ACTGTAACTCTGGTCTTCTCGTTCGCAACTGCACCATCACTGCCAATGCTGAGTGTGCCTGTCG
CAATGGCTGGCAGTGCAGGGACAAGGAGTGCACCGAGTGTGATCCTCTTCCAAACCCTTCGCTG
ACCGCTCGGTCGTCTCAGGCCCTGAGCCCACACCCTCAGCCCACCCACTTACCTTATGTCAGTG
AGATGCTGGAGGCCAGGACAGCTGGGCACATGCAGACTCTGGCTGACTTCAGGCAGCTGCCTGC
CCGGACTCTCTCTACCCACTGGCCACCCCAAAGATCCCTGTGCAGCTCCGATTTTATTCGCTTT
TGGGTGCTGGTGGTGGTTGGTGGAGTCCTGGCTTGCTATAGCTTGCTAGTAACAGTGGCCTTTA
TTATTTTCTGGGTG
SEQ ID NO: 2 - Exemplary amino acid sequence
comprising a CD27 CAR construct targeting a
CD70 antigen.
(SEQ ID NO: 2)
MARPHPWWLCVLGTLVGLSATPAPKSCPERHYWAQGKLCCQMCEPGTFLVKDCDOHRKAAQCDP
CIPGVSFSPDHHTRPHCESCRHCNSGLLVRNCTITANAECACRNGWQCRDKECTECDPLPNPSL
TARSSQALSPHPQPTHLPYVSEMLEARTAGHMQTLADFRQLPARTLSTHWPPQRSLCSSDFIRF
WVLVVVGGVLACYSLLVTVAFIIFWV
SEQ ID NO: 3 - Exemplary polynucleotide sequence
comprising a CD27 CAR construct targeting a CD70
antigen.
(SEQ ID NO: 3)
ATGGCACGGCCACATCCCTGGTGGCTGTGCGTTCTGGGGACCCTGGTGGGGCTCTCAGCTACTC
CAGCCCCCAAGAGCTGCCCAGAGAGGCACTACTGGGCTCAGGGAAAGCTGTGCTGCCAGATGTG
TGAGCCAGGAACATTCCTCGTGAAGGACTGTGACCAGCATAGAAAGGCTGCTCAGTGTGATCCT
TGCATACCGGGGGTCTCCTTCTCTCCTGACCACCACACCCGGCCCCACTGTGAGAGCTGTCGGC
ACTGTAACTCTGGTCTTCTCGTTCGCAACTGCACCATCACTGCCAATGCTGAGTGTGCCTGTCG
CAATGGCTGGCAGTGCAGGGACAAGGAGTGCACCGAGTGTGATCCTCTTCCAAACCCTTCGCTG
ACCGCTCGGTCGTCTCAGGCCCTGAGCCCACACCCTCAGCCCACCCACTTACCTTATGTCAGTG
AGATGCTGGAGGCCAGGACAGCTGGGCACATGCAGACTCTGGCTGACTTCAGGCAGCTGCCTGC
CCGGACTCTCTCTACCCACTGGCCACCCCAAAGATCCCTGTGCAGCTCCGATTTTATTCGCTTT
TGGGTGCTGGTGGTGGTTGGTGGAGTCCTGGCTTGCTATAGCTTGCTAGTAACAGTGGCCTTTA
TTATTTTCTGGGTGAGGAGTAAGAGGAGCAGGCTCCTGCACAGTGACTACATGAACATGACTCC
CCGCCGCCCCGGGCCCACCCGCAAGCATTACCAGCCCTATGCCCCACCACGCGACTTCGCAGCC
TATCGCTCACGCGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAAAGACGTGG
CCGGGACCCTGAGATGGGGGGAAAGCCGAGAAGGAAGAACCCTCAGGAAGGCCTGTACAATGAA
CTGCAGAAAGATAAGATGGCGGAGGCCTACAGTGAGATTGGGATGAAAGGCGAGCGCCGGAGGG
GCAAGGGGCACGATGGCCTTTACCAGGGTCTCAGTACAGCCACCAAGGACACCTACGACGCCCT
TCACATGCAGGCCCTGCCCCCTCGC
SEQ ID NO: 4 - Exemplary polynucleotide sequence
comprising a CD27 CAR construct targeting a CD70
antigen and IL-15.
(SEQ ID NO: 4)
ATGGCACGGCCACATCCCTGGTGGCTGTGCGTTCTGGGGACCCTGGTGGGGCTCTCAGCTACTC
CAGCCCCCAAGAGCTGCCCAGAGAGGCACTACTGGGCTCAGGGAAAGCTGTGCTGCCAGATGTG
TGAGCCAGGAACATTCCTCGTGAAGGACTGTGACCAGCATAGAAAGGCTGCTCAGTGTGATCCT
TGCATACCGGGGGTCTCCTTCTCTCCTGACCACCACACCCGGCCCCACTGTGAGAGCTGTCGGC
ACTGTAACTCTGGTCTTCTCGTTCGCAACTGCACCATCACTGCCAATGCTGAGTGTGCCTGTCG
CAATGGCTGGCAGTGCAGGGACAAGGAGTGCACCGAGTGTGATCCTCTTCCAAACCCTTCGCTG
ACCGCTCGGTCGTCTCAGGCCCTGAGCCCACACCCTCAGCCCACCCACTTACCTTATGTCAGTG
AGATGCTGGAGGCCAGGACAGCTGGGCACATGCAGACTCTGGCTGACTTCAGGCAGCTGCCTGC
CCGGACTCTCTCTACCCACTGGCCACCCCAAAGATCCCTGTGCAGCTCCGATTTTATTCGCTTT
TGGGTGCTGGTGGTGGTTGGTGGAGTCCTGGCTTGCTATAGCTTGCTAGTAACAGTGGCCTTTA
TTATTTTCTGGGTGAGGAGTAAGAGGAGCAGGCTCCTGCACAGTGACTACATGAACATGACTCC
CCGCCGCCCCGGGCCCACCCGCAAGCATTACCAGCCCTATGCCCCACCACGCGACTTCGCAGCC
TATCGCTCACGCGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAAAGACGTGG
CCGGGACCCTGAGATGGGGGGAAAGCCGAGAAGGAAGAACCCTCAGGAAGGCCTGTACAATGAA
CTGCAGAAAGATAAGATGGCGGAGGCCTACAGTGAGATTGGGATGAAAGGCGAGCGCCGGAGGG
GCAAGGGGCACGATGGCCTTTACCAGGGTCTCAGTACAGCCACCAAGGACACCTACGACGCCCT
TCACATGCAGGCCCTGCCCCCTCGCGGACCGCAGTGTACTAATTATGCTCTCTTGAAATTGGCT
GGAGATGTTGAGAGCAATCCCGGGCCCATGCGCATTAGCAAGCCCCACCTGCGGAGCATCAGCA
TCCAGTGCTACCTGTGCCTGCTGCTGAACAGCCACTTCCTGACCGAGGCCGGCATCCACGTGTT
CATCCTGGGCTGCTTCAGCGCCGGACTGCCCAAGACCGAGGCCAACTGGGTGAACGTGATCAGC
GACCTGAAGAAGATCGAGGACCTGATCCAGAGCATGCACATCGACGCCACCCTGTACACCGAGA
GCGACGTGCACCCCAGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGATCATCCTGGCCAAC
AACAGCCTGAGCAGCAACGGCAACGTGACCGAGAGCGGCTGCAAAGAGTGCGAGGAACTGGAAG
AGAAGAACATCAAAGAGTTTCTGCAGAGCTTCGTGCACATCGTGCAGATGTTCATCAACACCAG
CTGA
SEQ ID NO: 5 - Exemplary amino acid sequence
comprising a CD27 CAR construct targeting a
CD70 antigen.
(SEQ ID NO: 5)
MARPHPWWLCVLGTLVGLSATPAPKSCPERHYWAQGKLCCQMCEPGTFLVKDCDQHRKAAQCDP
CIPGVSFSPDHHTRPHCESCRHCNSGLLVRNCTITANAECACRNGWQCRDKECTECDPLPNPSL
TARSSQALSPHPQPTHLPYVSEMLEARTAGHMQTLADFRQLPARTLSTHWPPQRSLCSSDFIRE
WVLVVVGGVLACYSLLVTVAFIIFWVRSKRSRLLHSDYMNMTPRRPGPTRKHYQPYAPPRDFAA
YRSRVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 6 - Exemplary polynucleotide sequence
comprising a TROP2 CAR construct.
(SEQ ID NO: 6)
ATGGAGTTTGGGCTGAGCTGGCTTTTTCTTGTGGCTATTTTAAAAGGTGTCCAGTGCTCTAGAG
AGGACATCCAGCTGACCCAGTCTCCATCCTCCCTGTCTGCATCTGTAGGAGACAGAGTCAGCAT
CACCTGCAAGGCCAGTCAGGATGTGAGTATTGCTGTAGCCTGGTATCAGCAGAAACCAGGGAAA
GCCCCTAAGCTCCTGATCTACTCGGCATCCTACCGGTACACTGGAGTCCCTGATAGGTTCAGTG
GCAGTGGATCTGGGACAGATTTCACTCTCACCATCAGCAGTCTGCAACCTGAAGATTTTGCAGT
TTATTACTGTCAGCAACATTATATTACTCCGCTCACGTTCGGTGCTGGGACCAAGGTGGAGATC
AAACGTTTGGAAATAAAGGGCTCTACAAGCGGCTCAGGAAAACCTGGATCAGGCGAAGGGTCTA
CGCAGGTCCAACTGCAGCAATCTGGGTCTGAGTTGAAGAAGCCTGGGGCCTCAGTGAAGGTTTC
CTGCAAGGCTTCTGGATACACCTTCACAAACTATGGAATGAACTGGGTGAAGCAGGCCCCTGGA
CAAGGGCTTAAATGGATGGGCTGGATAAACACCTACACTGGAGAGCCAACATATACTGATGACT
TCAAGGGACGGTTTGCCTTCTCCTTGGACACCTCTGTCAGCACGGCATATCTCCAGATCAGCAG
CCTAAAGGCTGACGACACTGCCGTGTATTTCTGTGCAAGAGGGGGGTTCGGTAGTAGCTACTGG
TACTTCGATGTCTGGGGCCAAGGGTCCCTGGTCACCGTCTCCTCACCGTACGCCATTGAAGTTA
TGTATCCTCCTCCTTACCTAGACAATGAGAAGAGCAATGGAACCATTATCCATGTGAAAGGGAA
ACACCTTTGTCCAAGTCCCCTATTTCCCGGACCTTCTAAGCCCAAATTTTGGGTGCTGGTGGTG
GTTGGTGGAGTCCTGGCTTGCTATAGCTTGCTAGTAACAGTGGCCTTTATTATTTTCTGGGTGA
GGAGTAAGAGGAGCAGGCTCCTGCACAGTGACTACATGAACATGACTCCCCGCCGCCCCGGGCC
CACCCGCAAGCATTACCAGCCCTATGCCCCACCACGCGACTTCGCAGCCTATCGCTCACGCGTG
AAGTTCAGCAGGAGCGCAGACGCCCCCGCGTACCAGCAGGGCCAGAACCAGCTCTATAACGAGC
TCAATCTAGGACGAAGAGAGGAGTACGATGTTTTGGACAAAAGACGTGGCCGGGACCCTGAGAT
GGGGGGAAAGCCGAGAAGGAAGAACCCTCAGGAAGGCCTGTACAATGAACTGCAGAAAGATAAG
ATGGCGGAGGCCTACAGTGAGATTGGGATGAAAGGCGAGCGCCGGAGGGGCAAGGGGCACGATG
GCCTTTACCAGGGTCTCAGTACAGCCACCAAGGACACCTACGACGCCCTTCACATGCAGGCCCT
GCCCCCTCGCGGA
SEQ ID NO: 7 - Exemplary amino acid sequence
comprising a TROP2 CAR construct.
(SEQ ID NO: 7)
MEFGLSWLFLVAILKGVQCSREDIQLTQSPSSLSASVGDRVSITCKASQDVSIAVAWYQQKPGK
APKLLIYSASYRYTGVPDRFSGSGSGTDFTLTISSLQPEDFAVYYCQQHYITPLTFGAGTKVEI
KRLEIKGSTSGSGKPGSGEGSTQVQLQQSGSELKKPGASVKVSCKASGYTFTNYGMNWVKQAPG
QGLKWMGWINTYTGEPTYTDDFKGRFAFSLDTSVSTAYLQISSLKADDTAVYFCARGGFGSSYW
YFDVWGQGSLVTVSSPYAIEVMYPPPYLDNEKSNGTIIHVKGKHLCPSPLFPGPSKPKFWVLVV
VGGVLACYSLLVTVAFIIFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPRG
SEQ ID NO: 8 - Exemplary polynucleotide sequence
comprising a TROP2 CAR scFv.
(SEQ ID NO: 8)
ATGGAGTTTGGGCTGAGCTGGCTTTTTCTTGTGGCTATTTTAAAAGGTGTCCAGTGCTCTAGAG
AGGACATCCAGCTGACCCAGTCTCCATCCTCCCTGTCTGCATCTGTAGGAGACAGAGTCAGCAT
CACCTGCAAGGCCAGTCAGGATGTGAGTATTGCTGTAGCCTGGTATCAGCAGAAACCAGGGAAA
GCCCCTAAGCTCCTGATCTACTCGGCATCCTACCGGTACACTGGAGTCCCTGATAGGTTCAGTG
GCAGTGGATCTGGGACAGATTTCACTCTCACCATCAGCAGTCTGCAACCTGAAGATTTTGCAGT
TTATTACTGTCAGCAACATTATATTACTCCGCTCACGTTCGGTGCTGGGACCAAGGTGGAGATC
AAACGTTTGGAAATAAAGGGCTCTACAAGCGGCTCAGGAAAACCTGGATCAGGCGAAGGGTCTA
CGCAGGTCCAACTGCAGCAATCTGGGTCTGAGTTGAAGAAGCCTGGGGCCTCAGTGAAGGTTTC
CTGCAAGGCTTCTGGATACACCTTCACAAACTATGGAATGAACTGGGTGAAGCAGGCCCCTGGA
CAAGGGCTTAAATGGATGGGCTGGATAAACACCTACACTGGAGAGCCAACATATACTGATGACT
TCAAGGGACGGTTTGCCTTCTCCTTGGACACCTCTGTCAGCACGGCATATCTCCAGATCAGCAG
CCTAAAGGCTGACGACACTGCCGTGTATTTCTGTGCAAGAGGGGGGTTCGGTAGTAGCTACTGG
TACTTCGATGTCTGGGGCCAAGGGTCCCTGGTCACCGTCTCCTCA
SEQ ID NO: 9 - Exemplary amino acid sequence
comprising a TROP2 CAR scFv.
(SEQ ID NO: 9)
MEFGLSWLFLVAILKGVOCSREDIQLTQSPSSLSASVGDRVSITCKASQDVSIAVAWYQQKPGK
APKLLIYSASYRYTGVPDRFSGSGSGTDFTLTISSLOPEDFAVYYCQQHYITPLTFGAGTKVEI
KRLEIKGSTSGSGKPGSGEGSTQVOLQQSGSELKKPGASVKVSCKASGYTFTNYGMNWVKQAPG
QGLKWMGWINTYTGEPTYTDDFKGRFAFSLDTSVSTAYLQISSLKADDTAVYFCARGGFGSSYW
YFDVWGQGSLVTVSS
SEQ ID NO: 10 - Exemplary vector sequence
comprising iCas9, TROP2 CAR, and IL-15.
(SEQ ID NO: 10)
ATGCTCGAGGGAGTGCAGGTGGAAACCATCTCCCCAGGCGACGGGCGCACCTTCCCCAAGCGCG
GCCAGACCTGCGTGGTGCACTACACCGGGATGCTTGAAGATGGAAAGAAAGTTGATTCCTCCCG
GGACAGAAACAAGCCCTTTAAGTTTATGCTAGGCAAGCAGGAGGTGATCCGAGGCTGGGAAGAA
GGGGTTGCCCAGATGAGTGTGGGTCAGAGAGCCAAACTGACTATATCTCCAGATTATGCCTATG
GTGCCACTGGGCACCCAGGCATCATCCCACCACATGCCACTCTCGTCTTCGATGTGGAGCTTCT
AAAACTGGAATCTGGCGGTGGATCCGGAGTCGACGGATTTGGTGATGTCGGTGCTCTTGAGAGT
TTGAGGGGAAATGCAGATTTGGCTTACATCCTGAGCATGGAGCCCTGTGGCCACTGCCTCATTA
TCAACAATGTGAACTTCTGCCGTGAGTCCGGGCTCCGCACCCGCACTGGCTCCAACATCGACTG
TGAGAAGTTGCGGCGTCGCTTCTCCTCGCTGCATTTCATGGTGGAGGTGAAGGGCGACCTGACT
GCCAAGAAAATGGTGCTGGCTTTGCTGGAGCTGGCGCAGCAGGACCACGGTGCTCTGGACTGCT
GCGTGGTGGTCATTCTCTCTCACGGCTGTCAGGCCAGCCACCTGCAGTTCCCAGGGGCTGTCTA
CGGCACAGATGGATGCCCTGTGTCGGTCGAGAAGATTGTGAACATCTTCAATGGGACCAGCTGC
CCCAGCCTGGGAGGGAAGCCCAAGCTCTTTTTCATCCAGGCCTGTGGTGGGGAGCAGAAAGATC
ATGGGTTTGAGGTGGCCTCCACTTCCCCTGAAGACGAGTCCCCTGGCAGTAACCCCGAGCCAGA
TGCCACCCCGTTCCAGGAAGGTTTGAGGACCTTCGACCAGCTGGACGCCATATCTAGTTTGCCC
ACACCCAGTGACATCTTTGTGTCCTACTCTACTTTCCCAGGTTTTGTTTCCTGGAGGGACCCCA
AGAGTGGCTCCTGGTACGTTGAGACCCTGGACGACATCTTTGAGCAGTGGGCTCACTCTGAAGA
CCTGCAGTCCCTCCTGCTTAGGGTCGCTAATGCTGTTTCGGTGAAAGGGATTTATAAACAGATG
CCTGGTTGCTTTAATTTCCTCCGGAAAAAACTTTTCTTTAAAACATCAGCTTCGCGAGCCGAGG
GCAGGGGAAGTCTTCTAACATGCGGGGACGTGGAGGAAAATCCCGGGCCCATGGAGTTTGGGCT
GAGCTGGCTTTTTCTTGTGGCTATTTTAAAAGGTGTCCAGTGCTCTAGAGAGGACATCCAGCTG
ACCCAGTCTCCATCCTCCCTGTCTGCATCTGTAGGAGACAGAGTCAGCATCACCTGCAAGGCCA
GTCAGGATGTGAGTATTGCTGTAGCCTGGTATCAGCAGAAACCAGGGAAAGCCCCTAAGCTCCT
GATCTACTCGGCATCCTACCGGTACACTGGAGTCCCTGATAGGTTCAGTGGCAGTGGATCTGGG
ACAGATTTCACTCTCACCATCAGCAGTCTGCAACCTGAAGATTTTGCAGTTTATTACTGTCAGC
AACATTATATTACTCCGCTCACGTTCGGTGCTGGGACCAAGGTGGAGATCAAACGTTTGGAAAT
AAAGGGCTCTACAAGCGGCTCAGGAAAACCTGGATCAGGCGAAGGGTCTACGCAGGTCCAACTG
CAGCAATCTGGGTCTGAGTTGAAGAAGCCTGGGGCCTCAGTGAAGGTTTCCTGCAAGGCTTCTG
GATACACCTTCACAAACTATGGAATGAACTGGGTGAAGCAGGCCCCTGGACAAGGGCTTAAATG
GATGGGCTGGATAAACACCTACACTGGAGAGCCAACATATACTGATGACTTCAAGGGACGGTTT
GCCTTCTCCTTGGACACCTCTGTCAGCACGGCATATCTCCAGATCAGCAGCCTAAAGGCTGACG
ACACTGCCGTGTATTTCTGTGCAAGAGGGGGGTTCGGTAGTAGCTACTGGTACTTCGATGTCTG
GGGCCAAGGGTCCCTGGTCACCGTCTCCTCACCGTACGCCATTGAAGTTATGTATCCTCCTCCT
TACCTAGACAATGAGAAGAGCAATGGAACCATTATCCATGTGAAAGGGAAACACCTTTGTCCAA
GTCCCCTATTTCCCGGACCTTCTAAGCCCAAATTTTGGGTGCTGGTGGTGGTTGGTGGAGTCCT
GGCTTGCTATAGCTTGCTAGTAACAGTGGCCTTTATTATTTTCTGGGTGAGGAGTAAGAGGAGC
AGGCTCCTGCACAGTGACTACATGAACATGACTCCCCGCCGCCCCGGGCCCACCCGCAAGCATT
ACCAGCCCTATGCCCCACCACGCGACTTCGCAGCCTATCGCTCACGCGTGAAGTTCAGCAGGAG
CGCAGACGCCCCCGCGTACCAGCAGGGCCAGAACCAGCTCTATAACGAGCTCAATCTAGGACGA
AGAGAGGAGTACGATGTTTTGGACAAAAGACGTGGCCGGGACCCTGAGATGGGGGGAAAGCCGA
GAAGGAAGAACCCTCAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCCTCGCGGAC
CGCAGTGTACTAATTATGCTCTCTTGAAATTGGCTGGAGATGTTGAGAGCAATCCCGGGCCCAT
GCGCATTAGCAAGCCCCACCTGCGGAGCATCAGCATCCAGTGCTACCTGTGCCTGCTGCTGAAC
AGCCACTTCCTGACCGAGGCCGGCATCCACGTGTTCATCCTGGGCTGCTTCAGCGCCGGACTGC
CCAAGACCGAGGCCAACTGGGTGAACGTGATCAGCGACCTGAAGAAGATCGAGGACCTGATCCA
GAGCATGCACATCGACGCCACCCTGTACACCGAGAGCGACGTGCACCCCAGCTGCAAGGTGACC
GCCATGAAGTGCTTTCTGCTGGAACTGCAGGTGATCAGCCTGGAAAGCGGCGACGCCAGCATCC
ACGACACCGTGGAGAACCTGATCATCCTGGCCAACAACAGCCTGAGCAGCAACGGCAACGTGAC
CGAGAGCGGCTGCAAAGAGTGCGAGGAACTGGAAGAGAAGAACATCAAAGAGTTTCTGCAGAGC
TTCGTGCACATCGTGCAGATGTTCATCAACACCAGCTGA

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells of any kind as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV. Non-viral vectors include plasmids, transposons, nanoparticles, liposome, lipids, metals, or a combination thereof.

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form. In some embodiments, a TCR and/or CD3 complex is as described in International Application PCT/US2022/074062 filed Jul. 22, 2022, and published as WO2023004425A2 on Jan. 26, 2023, which is hereby incorporated by reference in it's entirety for the purposes described herein.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3εchains, and a homodimer of CD3ζ chains. The CD3γ, CD3δ, and CD3δ chains are highly related cell surface proteins of the immunoglobulin superfamily containing a single immunoglobulin domain. The transmembrane regions of the CD3γ, CD3δ, and CD3δ chains are negatively charged, which is a characteristic that allows these chains to associate with the positively charged T cell receptor chains. The intracellular tails of the CD3γ, CD3δ, and CD3δ chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

The CARs and TCRs of the disclosure target one or more particular antigens. Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, EBNA, HER2, CA-125, TRAIL/DR4, CD20, CD70, CD3δ, trop2, HLA-G, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, β-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6,-10, GAGE-1,-2,-8, GAGE-3,-4,-5,-6,-7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP,-catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, and LRRN1. Examples of sequences for antigens are known in the art, for example, in the GenBank® database: CD19 (Accession No. NG_007275.1), EBNA (Accession No. NG_002392.2), WT1 (Accession No. NG_009272.1), CD123 (Accession No. NC 000023.11:1336785-1382689), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### 2. Suicide Gene

In particular embodiments, a suicide gene is utilized in conjunction with the antibody combination therapy to control its use and allow for termination of the cell therapy at a desired event and/or time. The suicide gene is employed in transduced cells for the purpose of eliciting death for the transduced cells when needed. The cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α, γ-lyase (MET), and Thymidine phosphorylase (TP), as examples.

In particular embodiments, vectors that encode the CAR, or any vector in a NK cell encompassed herein, include one or more suicide genes. The suicide gene may or may not be on the same vector as a CAR. In cases wherein the suicide gene is present on the same vector as the CAR, the suicide gene and the CAR may be separated by an IRES or 2A element, for example.

### 3. Cytokines

In some embodiments, the cells expressing the NK cells are engineered to express one or more heterologous cytokines and/or are engineered to upregulate normal expression of one or more heterologous cytokines. The cells may or may not be transduced or transfected for one or more cytokines on the same vector as other genes.

One or more cytokines may be co-expressed from a vector, including as a separate polypeptide from the engineered antigen receptor and/or suicide gene. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines (e.g., IL-2, IL-12, IL-18, and/or IL-21), chemokines, and other molecules that contribute to the activation and proliferation of cells used for human application. In certain embodiments, NK cells expressing IL-15 are capable of continued supportive cytokine signaling, which is useful for their survival post-infusion. In certain embodiments, NK cells expressing IL-21 are capable of continued supportive cytokine signaling, which is useful for their survival post-infusion. In certain embodiments, a cytokine is expressed as part of a multicistronic construct with one or more functional proteins and/or marker proteins.

In some embodiments, a specific sequence of IL-15 is utilized, such as those that follow (underlining refers to signal peptide sequence, which may be included or omitted):

SEQ ID NO: 11 - Exemplary polynucleotide
sequence comprising IL-15 (signal peptide
underlined).
(SEQ ID NO: 11)
ATGCGCATTAGCAAGCCCCACCTGCGGAGCATCAGCATCCAGTGCTACCT
GTGCCTGCTGCTGAACAGCCACTTCCTGACCGAGGCCGGCATCCACGTGT
TCATCCTGGGCTGCTTCAGCGCCGGACTGCCCAAGACCGAGGCCAACTGG
GTGAACGTGATCAGCGACCTGAAGAAGATCGAGGACCTGATCCAGAGCAT
GCACATCGACGCCACCCTGTACACCGAGAGCGACGTGCACCCCAGCTGCA
AGGTGACCGCCATGAAGTGCTTTCTGCTGGAACTGCAGGTGATCAGCCTG
GAAAGCGGCGACGCCAGCATCCACGACACCGTGGAGAACCTGATCATCCT
GGCCAACAACAGCCTGAGCAGCAACGGCAACGTGACCGAGAGCGGCTGCA
AAGAGTGCGAGGAACTGGAAGAGAAGAACATCAAAGAGTTTCTGCAGAGC
TTCGTGCACATCGTGCAGATGTTCATCAACACCAGC
SEQ ID NO: 12 - Exemplary amino acid
sequence comprising a comprising IL-15
(signal peptide underlined).
(SEQ ID NO: 12)
MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS

In some embodiments, a specific sequence of IL-21 is utilized, such as those that follow (underlining refers to signal peptide sequence, which may be included or omitted):

SEQ ID NO: 13 - Exemplary codon optimized
polynucleotide sequence comprising IL-21
(signal peptide underlined).
(SEQ ID NO: 13)
ATGAGGAGCAGTCCAGGCAATATGGAGCGGATAGTAATTTGTCTCATGGT
AATATTCCTCGGTACTCTGGTACATAAATCTTCCTCTCAAGGTCAGGACC
GCCATATGATTCGAATGCGGCAGCTGATTGACATAGTCGATCAACTGAAG
AACTATGTGAATGATCTTGTGCCCGAGTTTTTGCCAGCCCCTGAAGACGT
AGAAACTAATTGTGAGTGGAGTGCCTTTTCCTGCTTTCAAAAGGCACAGC
TGAAATCCGCCAACACGGGCAATAACGAACGGATAATTAACGTATCCATT
AAGAAGCTGAAGCGGAAGCCGCCCTCAACCAATGCGGGACGGCGGCAAAA
GCATCGCTTGACCTGTCCGTCATGCGACAGCTACGAGAAAAAGCCCCCGA
AGGAGTTCTTGGAACGCTTCAAGAGTCTCCTTCAGAAAATGATTCACCAG
CACCTGTCCTCACGGACGCACGGAAGCGAGGACAGT
SEQ ID NO: 14 - Exemplary codon optimized
amino acid sequence comprising a comprising
IL-21 (signal peptide underlined).
(SEQ ID NO: 14)
MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS
SEQ ID NO: 15 - Exemplary polynucleotide
sequence comprising IL-21.
(SEQ ID NO: 15)
GGCCAGGACCGGCACATGATCCGGATGAGACAGCTGATCGACATCGTGGA
CCAGCTGAAGAACTACGTGAACGACCTGGTGCCCGAGTTCCTGCCTGCCC
CCGAGGACGTGGAAACAAACTGCGAGTGGAGCGCCTTCAGCTGCTTCCAG
AAGGCCCAGCTGAAAAGCGCCAACACCGGCAACAACGAGCGGATCATCAA
CGTGTCCATCAAGAAGCTGAAGCGGAAGCCCCCCAGCACCAACGCCGGAA
GAAGGCAGAAGCACAGACTGACCTGCCCCAGCTGCGACAGCTACGAGAAG
AAGCCCCCTAAAGAGTTCCTGGAACGGTTCAAGAGCCTGCTGCAGAAGAT
GATCCACCAGCACCTGAGCAGCCGGACCCACGGCTCTGAGGACAGC
SEQ ID NO: 16 - Exemplary amino acid
sequence comprising a comprising IL-21.
(SEQ ID NO: 16)
GQDRHMIRMRQLIDIVDQLKNYVNDLVPEFLPAPEDVETNCEWSAFSCFQ
KAQLKSANTGNNERIINVSIKKLKRKPPSTNAGRROKHRLTCPSCDSYEK
KPPKEFLERFKSLLQKMIHQHLSSRTHGSEDS

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene and/or as the CAR. In some embodiments, the present disclosure concerns co-utilization of a CAR with IL-15 and optionally a suicide gene.

In some embodiments, the cells express one or more exogenously provided engineered receptors, wherein the engineered receptor comprises a chemokine receptor and/or a cytokine receptor. In some embodiments, a cytokine receptor is an IL-15 receptor. In some embodiments, a cytokine receptor is a non-naturally occurring variant of a cytokine receptor. In some embodiments, a cytokine receptor is an IL-15, IL-12, IL-2, IL-18, IL-21, IL-23, or GMCSF receptor, or a combination thereof.

### 4. Knockout or Knockdown of Endogenous Genes

The NK cell production process of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous antigen receptors, whereas in other cases the gene editing occurs in NK cells that do not express a heterologous antigen receptor but that ultimately will express one or more heterologous antigen receptors, in at least some cases. In particular embodiments, the NK cells that are gene edited are expanded NK cells.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, GR, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL1OR, TDAG8, CD5, CD7, SLAMF7, CD3δ, LAG3, TCR, beta2-microglobulin, HLA, CD73, CREB, CREM, ICER, and CD39. In specific embodiments, the TGFBR2 gene is knocked out or knocked down in the NK cells. In specific embodiments, the CISH gene is knocked out or knocked down in the NK cells. In specific embodiments, the CD3δ gene is knocked out or knocked down in the NK cells. In specific embodiments, the Glucocorticoid receptor (GR) gene is knocked out or knocked down in the NK cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Methods of utilizing a CRISPR system are well known in the art.

### F. Cryopreservation

In particular cases, NK cells and/or antibodies of the disclosure are preserved in a cryopreservation medium composition comprising at least one cryoprotectant, a serum (human or animal serum) or a non-serum alternative to serum (not human serum or animal serum), and at least one cytokine and/or at least one growth factor. In some cases, the cryoprotectant is dimethyl sulfoxide (DMSO), glycerin, glycerol, hydroxyethyl starch, or a combination thereof. The non-serum alternative may be of any kind, including at least platelet lysate and/or a blood product lysate (for example, human serum albumin). In embodiments of the composition wherein one or more (including two or more) cytokines are utilized, the cytokine may be a natural or a recombinant or a synthetic protein. At least one of the cytokines may be an Food and Drug Administration (FDA)—approved cytokine. Examples of cytokines and growth factors include at least IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, thrombopoietin, erythropoietin, or a combination thereof. For serum embodiments, the serum may be an animal-derived serum, such as human serum (including human AB serum) or bovine serum. DMSO and other cryoprotectants, when utilized may comprise 4-10%, 4-6%, 4-8%, 5-10%, 5-8%, 6-10%, 6-8%, 8-10%, and so forth, of the composition. For embodiments wherein serum is employed, the serum may comprise 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of serum. In specific embodiments, the composition comprises platelet lysate that may be at any concentration in the composition, but in certain embodiments the platelet lysate comprises 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of platelet lysate.

The composition may have certain concentrations of components, including cytokines and/or growth factors. In specific cases, any cytokine, including IL-2, IL-21, and/or IL-15, for example, are present in the composition in a particular concentration. The IL-2 may be present at a concentration of 1-5000, 1-1000, 1-500, 1-100, 100-5000, 100-500, 500-5000, 500-1000, or 1000-5000 U/mL, for example. In a specific case, the IL-2 is present at a concentration in the composition of at least or no more than 100, 200, 300, 400, 500, 600, 700, 800, 900, or 1000 U/mL. In specific embodiments, IL-21 is present in the composition at a concentration of 10-3000, 10-2000, 10-1000, 10-500, 10-100, 100-3000, 100-2000, 100-1000, 500-3000, 500-2000, 500-1000, 1000-3000, 1000-2000, or 2000-3000 ng/ml. The IL-21 may be in a concentration in the composition of at least or nor more than 1, 5, 10, 20, 30, 40, 50, 60, 70, 80, 90, 100, 150, 200, 250, 300, 350, 400, 450, 500, 750, 1000, 1250, 1500, 1750, 2000, 2250, 2500, 2750, or 3000 ng/mL. IL-15 may be present in the composition at a concentration of 1-2000, 1-1000, 1-500, 1-100, 100-2000, 100-1000, 100-500, 500-2000, 500-1000, or 1000-2000 ng/ml. IL-15 may be present in the composition at a concentration of at least or no more than 10, 50, 100, 500, 1000, 1500, or 2000 ng/mL.

Compositions as encompassed herein that comprise at least one cryoprotectant, a serum or a non-serum alternative to serum, and at least one cytokine and/or at least one growth factor may further comprise a plurality of immune cells and/or stem cells, each of any kind. In specific embodiments, the cells are NK cells, T cells, B cells, NKT cells derived from mature bone marrow or peripheral blood cells, cell lines such as tumor cell lines (e.g., NK92 or other NK lines), hematopoietic stem cells, induced pluripotent stem cells, MSCs (a population of cells alternatively called “mesenchymal stem cells” and “mesenchymal stromal cells” in the literature), or a mixture thereof, which can be derived from bone marrow, peripheral blood, skin, adipose tissue, or a combination thereof. In embodiments wherein NK cells are utilized, the NK cells may or may not be expanded NK cells. Embodiments of the disclosure also encompass pharmaceutical compositions that comprise any composition of the disclosure and a suitable pharmaceutically acceptable carrier.

In certain embodiments, cells and/or antibodies are treated with one or more deactivating agents (e.g., a kinase inhibitor, e.g., Dasatinib, Nilotinib, Rapamycin, etc.) pre-cryopreservation.

In some embodiments, technologies described herein comprise deactivating a NK cell, comprising treating an NK cell with an effective amount of one or more deactivating agents under conditions to produce a deactivated NK cell. In some embodiments, a deactivating agent is a kinase inhibitor. In some embodiments, a deactivating agent is a mechanistic target of rapamycin (mTOR) inhibitor. In some embodiments, the mTOR inhibitor is rapamycin, everolimus, and/or temsirolimus. In some embodiments, the mTOR inhibitor is rapamycin. In some embodiments, the deactivating agent is a tyrosine kinase (TK) inhibitor. In some embodiments, the TK inhibitor is Lorlatinib, Brigatinib, Ceritinib, Alectinib, Crizotinib, Bosutinib, Ponatinib, Nilotinib, Dasatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib. In some embodiments, the TK inhibitor is a BCR-Abl inhibitor. In some embodiments, the TK inhibitor is Bosutinib, Ponatinib, Nilotinib, Dasatinib, and/or Imatinib. In some embodiments, the TK inhibitor is Dasatinib and/or Nilotinib. In some embodiments, the TK inhibitor is Dasatinib.

In some embodiments, treatment with a deactivating agent is at any point during culturing of the NK cell. In some embodiments, the treatment is for about 24 to about 96 hours, about 36 to about 84 hours, or about 48 to about 72 hours. In some embodiments, the treatment is for about 24 hours, about 48 hours, or about 72 hours. In some embodiments, the NK cell is treated with the deactivating agent at a concentration of about 1 to about 1000 nM. In some embodiments, the NK cell is treated with the deactivating agent at a concentration of about 5 to about 500 nM. In some embodiments, the NK cell is treated with the deactivating agent at a concentration of about 20 to about 200 nM. In some embodiments, the NK cell is treated with the deactivating agent at a concentration of about 30 to about 100 nM. In some embodiments, the deactivated NK cell has an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell.

In some embodiments, technologies described herein comprise methods of maintaining the viability of a population of cells over at least 50% percent following cryopreservation of the population, comprising the step of subjecting the population to an effective amount of one or more deactivating agents (e.g., a tyrosine kinase inhibitor) to deactivate the cells prior to cryopreservation, cryopreserving the cells, and thawing the population, wherein upon thawing the viability of the population is over at least 50%. In some cases, upon thawing of the cells the viability of the population of cells is over at least 55, 60, 65, 70, 75, 80, 85, 90, 91, 92, 93, 94, 95, 96, 97, 98, or 99% following cryopreservation of the population.

In specific embodiments, the cells of the disclosure at any step are preserved in the following particular formulations for cryopreservation media (see e.g., Table 1 and/or Table 2).

TABLE 1
Exemplary cryopreservation media

50% RPMI; 25% dextran; 20% human albumin, 5% DMSO
50% RPMI; 25% dextran; 20% human albumin, 5% DMSO + IL-2/IL-15
35% RPMI; 40% dextran; 20% human albumin, 5% DMSO
35% RPMI; 40% dextran; 20% human albumin, 5% DMSO + IL-2/IL-15
32.5% RPMI; 40% dextran; 20% human albumin, 7.5% DMSO
32.5% RPMI; 40% dextran; 20% human albumin, 7.5% DMSO + IL-2/IL-15
50% PlasmaLyte; 25% dextran; 20% human albumin, 5% DMSO
50% PlasmaLyte; 25% dextran; 20% human albumin, 5% DMSO + IL-2/IL-15
35% PlasmaLyte; 40% dextran; 20% human albumin, 5% DMSO
35% PlasmaLyte; 40% dextran; 20% human albumin, 5% DMSO + IL-2/IL-15
32.5% PlasmaLyte; 40% dextran; 20% human albumin, 7.5% DMSO
32.5% PlasmaLyte; 40% dextran; 20% human albumin, 7.5% DMSO + IL-2/IL-15
70% PlasmaLyte; 25% dextran; 5% DMSO + IL-2/IL-15
90% PLT Lys + 10% DMSO + IL-2/IL-15
50% PLT lys + 25% dextran + 20% human albumin + 5% DMSO + IL-2/IL-15
50% AB serum + 25% dextran + 20% human albumin + 5% DMSO + IL-2/IL-15
50% PLT lys + 25% dextran + 20% human albumin + 5% DMSO + IL-2/IL-21
50% RPMI + 25% dextran + 20% human albumin + 5% DMSO + IL-2/IL-21
50% PlasmaLyte + 25% dextran + 20% human albumin + 5% DMSO + IL-2/IL-21
50% AB serum + 25% dextran + 20% human albumin + 5% DMSO + IL-2/IL-21
50% PLT Lys; 25% Dextran in NACL; 20% human albumin; 5% DMSO + IL-2/IL-15
50% PLT Lys; 25% Dextran in Dextrose; 20% human albumin; 5% DMSO + IL-2/IL-15
25% PLT Lys; 50% Dextran in NACL; 20% human albumin; 5% DMSO + IL-2/IL-15
25% PLT Lys; 50% Dextran in Dextrose; 20% human albumin; 5% DMSO + IL-2/IL-15
25% Dextran in NACL; 70% human albumin; 5% DMSO + IL-2/IL-15
25% Dextran in Dextrose; 70% human albumin; 5% DMSO + IL-2/IL-15
50% Dextran in NACL; 45% human albumin; 5% DMSO + IL-2/IL-15
50% Dextran in Dextrose; 45% human albumin; 5% DMSO + IL-2/IL-15
50% PlasmaLyte; 45% human albumin; 5% DMSO + IL-2/IL-15
25% PlasmaLyte; 70% human albumin; 5% DMSO + IL-2/IL-15
Crystor Cell preservation Media CS10 (1% to 100%)
Crystor Cell preservation Media CS10 (1% to 100%) + IL-2/15

TABLE 2
Exemplary cryopreservation media with particular concentrations

50% Platelet lysate; 25% Dextran in NaCL; 20% human albumin; 5% DMSO; plus 10-200iu
of interleukin 2 and/or 1-10 ng/ml of interleukin 15
50% Platelet lysate; 25% Dextran in Dextrose; 20% human albumin; 5% DMSO; plus 10-
200iu of interleukin 2 and/or 1-10 ng/ml of interleukin 15
25% Platelet lysate; 50% Dextran in NaCL; 20% human albumin; 5% DMSO; plus 10-200iu
of interleukin 2 and/or 1-10 ng/ml of interleukin 15
25% Platelet lysate; 50% Dextran in Dextrose; 20% human albumin; 5% DMSO; plus 10-
200iu of interleukin 2 and/or 1-10 ng/ml of interleukin 15
25% Dextran in NaCL; 70% human albumin; 5% DMSO; plus 10-200iu of interleukin 2
and/or 1-10 ng/ml of interleukin 15
25% Dextran in Dextrose; 70% human albumin; 5% DMSO; plus 10-200iu of interleukin 2
and/or 1-10 ng/ml of interleukin 15
50% Dextran in NaCL; 45% human albumin; 5% DMSO; plus 10-200iu of interleukin 2
and/or 1-10 ng/ml of interleukin 15
50% Dextran in Dextrose; 45% human albumin; 5% DMSO; plus 10-200iu of interleukin 2
and/or 1-10 ng/ml of interleukin 15
50% PlasmaLyte; 45% human albumin; 5% DMSO; plus 10-200iu of interleukin 2 and/or 1-
10 ng/ml of interleukin 15
25% PlasmaLyte; 70% human albumin; 5% DMSO; plus 10-200iu of interleukin 2 and/or 1-
10 ng/ml of interleukin 15
90% Platelet lysate, 10% DMSO
90% Platelet lysate, 10% DMSO plus 10-200iu of interleukin 2 and/or 1-10 ng/ml of
interleukin 15
Crystor Cell preservation Media CS10 (1% to 100%)
Crystor Cell preservation Media CS10 (1% to 100%) + IL-2

### II. Methods of Use

Embodiments of the present disclosure concern methods for the use of the compositions comprising NK cells (e.g., engineered NK cells) and antibodies provided herein for treating or preventing a medical disease or disorder. The method includes administering to the subject a therapeutically effective amount of the loaded, optionally pre-activated, and optionally expanded NK cells with the antibodies, thereby treating or preventing the disease in the subject, including reducing the risk of, reducing the severity of, and/or delaying the onset of the disease. In certain embodiments of the present disclosure, cancer or infection is treated by transfer of a composition comprising the NK cell population and antibodies. In at least some cases, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy.

Cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

In certain embodiments, cancers for which the present compositions and methods described herein are useful for treatment, prevention, and/or amelioration of symptoms include at least EGFR, EGFR2, and/or c-MET expressing cancers. In certain embodiments, cancers for which the present compositions and methods described herein are useful for treatment, prevention, and/or amelioration of symptoms include at least CD70, and/or TROP2 expressing cancers. In certain embodiments, compositions and methods described herein are utilized for treatment, prevention, and/or amelioration of symptoms associated with PDAC. In certain embodiments, compositions and methods described herein are utilized for treatment, prevention, and/or amelioration of symptoms associated with CRC. In certain embodiments, compositions and methods described herein are utilized for treatment, prevention, and/or amelioration of symptoms associated with kidney cancer. In certain embodiments, compositions and methods described herein are utilized for treatment, prevention, and/or amelioration of symptoms associated with glioblastoma. In certain embodiments, compositions and methods described herein are utilized for treatment, prevention, and/or amelioration of symptoms associated with breast cancer. In certain embodiments, compositions and methods described herein are utilized for treatment, prevention, and/or amelioration of symptoms associated with RCC. In certain embodiments, compositions and methods described herein are utilized for treatment, prevention, and/or amelioration of symptoms associated with myeloma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; β-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

Acute leukemia is characterized by the rapid proliferation of immature blood cells. This crowding makes the bone marrow unable to produce healthy blood cells. Acute forms of leukemia can occur in children and young adults. In fact, it is a more common cause of death for children in the U.S. than any other type of malignant disease. Immediate treatment is required in acute leukemia due to the rapid progression and accumulation of the malignant cells, which then spill over into the bloodstream and spread to other organs of the body. Central nervous system (CNS) involvement is uncommon, although the disease can occasionally cause cranial nerve palsies. Chronic leukemia is distinguished by the excessive buildup of relatively mature, but still abnormal, blood cells. Typically taking months to years to progress, the cells are produced at a much higher rate than normal cells, resulting in many abnormal white blood cells in the blood. Chronic leukemia mostly occurs in older people, but can theoretically occur in any age group. Whereas acute leukemia must be treated immediately, chronic forms are sometimes monitored for some time before treatment to ensure maximum effectiveness of therapy.

Furthermore, the diseases are classified into lymphocytic or lymphoblastic, which indicate that the cancerous change took place in a type of marrow cell that normally goes on to form lymphocytes, and myelogenous or myeloid, which indicate that the cancerous change took place in a type of marrow cell that normally goes on to form red cells, some types of white cells, and platelets (see lymphoid cells vs. myeloid cells).

Acute lymphocytic leukemia (also known as acute lymphoblastic leukemia, or ALL) is the most common type of leukemia in young children. This disease also affects adults, especially those aged 65 and older. Chronic lymphocytic leukemia (CLL) most often affects adults over the age of 55. It sometimes occurs in younger adults, but it almost never affects children. Acute myelogenous leukemia (also known as acute myeloid leukemia, or AML) occurs more commonly in adults than in children. This type of leukemia was previously called “acute nonlymphocytic leukemia.” Chronic myelogenous leukemia (CML) occurs mainly in adults. A very small number of children also develop this disease.

Lymphoma is a type of cancer that originates in lymphocytes (a type of white blood cell in the vertebrate immune system). There are many types of lymphoma. According to the U.S. National Institutes of Health, lymphomas account for about five percent of all cases of cancer in the United States, and Hodgkin's lymphoma in particular accounts for less than one percent of all cases of cancer in the United States. Because the lymphatic system is part of the body's immune system, patients with a weakened immune system, such as from HIV infection or from certain drugs or medication, also have a higher incidence of lymphoma.

In certain embodiments of the disclosure, compositions comprising the NK cells and antibodies are delivered to an individual in need thereof, such as an individual that has cancer or an infection. In at least some cases, the cells can enhance the individual's immune system to attack the respective cancer or pathogenic cells. In some cases, the individual is provided with one or more doses of the compositions comprising the NK cells and antibodies. In cases where the individual is provided with two or more doses of the NK cells/antibodies, the duration between the administrations should be sufficient to allow time for propagation in the individual, and in specific embodiments the duration between doses is 1, 2, 3, 4, 5, 6, 7, or more days.

The source of NK cells that are pre-activated (optionally) and expanded may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹² cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹² cells within several weeks of initiating an intra-patient dose escalation scheme. Suitable modes of administration include intravenous, subcutaneous, intracavitary (for example by reservoir-access device), intraperitoneal, and direct injection into a tumor mass.

The compositions comprising the NK cells and antibodies generated according to the present methods have many potential uses, including experimental and therapeutic uses. In particular, it is envisaged that such cell populations are useful in suppressing undesirable or inappropriate immune responses. In such methods, a small number of NK cells are removed from a patient and then manipulated and expanded ex vivo before reinfusing them into the patient. Examples of diseases which may be treated in this way are autoimmune diseases and conditions in which suppressed immune activity is desirable, e.g., for allo-transplantation tolerance. A therapeutic method could comprise obtaining NK cells from a mammal; expanding the NK cells ex vivo in accordance with the methods of the present methods as described herein; exposing under sufficient conditions the NK cells to the antibodies; and administering the compositions comprising the expanded NK cells/antibodies to the mammal to be treated.

A pharmaceutical composition of the present disclosure can be used alone or in combination with other well-established agents useful for treating cancer. Whether delivered alone or in combination with other agents, the pharmaceutical composition of the present disclosure can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and expanded NK cells/antibodies of the disclosure are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of NK cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. NK cells, such as immunosuppressive CD19+ cells, can be administered along with the antibodies prior to transplantation, concurrently with transplantation, and/or following transplantation. In some embodiments, the compositions comprising the NK cells and antibodies are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of the compositions comprising the NK cells and antibodies occurs 3-5 days prior to transplantation.

NK cells/antibodies administered to a patient that is receiving a transplant can be sensitized with antigens specific to the transplanted material prior to administration. According to this embodiment, the transplant recipient will have a decreased immune/inflammatory response to the transplanted material and, as such, the likelihood of rejection of the transplanted tissue is minimized. Similarly, with regard to the treatment of graft versus host disease, the NK cells can be sensitized with antigens specific to the host. According to this embodiment, the recipient will have a decreased immune/inflammatory response to self-antigens.

In a further embodiment, administration of a therapeutically effective amount of compositions comprising NK cells and antibodies to a subject treats or inhibits inflammation in the subject. Thus, the method includes administering a therapeutically effective amount of compositions comprising NK cells and antibodies to the subject to inhibit the inflammatory process. Examples of inflammatory disorders include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections. The methods disclosed herein can also be used to treat allergic disorders.

Administration of compositions comprising NK cells and antibodies can be utilized whenever immunosuppression or inhibition of inflammation is desired, for example, at the first sign or symptoms of a disease or inflammation. These may be general, such as pain, edema, elevated temperature, or may be specific signs or symptoms related to dysfunction of affected organ(s). For example, in renal transplant rejection there may be an elevated serum creatinine level, whereas in GVHD, there may be a rash, and in asthma, there may be shortness of breath and wheezing.

Administration of compositions comprising NK cells and antibodies can also be utilized to prevent immune-mediated disease in a subject of interest. For example, compositions comprising NK cells and antibodies can be administered to a subject that will be a transplant recipient prior to the transplantation. In another example, compositions comprising NK cells and antibodies are administered to a subject receiving allogeneic bone marrow transplants without T cell depletion. In a further example, compositions comprising NK cells and antibodies can be administered to a subject with a family history of diabetes. In other example, compositions comprising NK cells and antibodies are administered to a subject with asthma in order to prevent an asthma attack. In some embodiments, a therapeutically effective amount of compositions comprising NK cells and antibodies is administered to the subject in advance of a symptom. The administration of the compositions comprising NK cells and antibodies results in decreased incidence or severity of subsequent immunological event or symptom (such as an asthma attack), or improved patient survival, compared to patients who received other therapy not including compositions comprising NK cells and antibodies.

In certain embodiments, the compositions comprising NK cells and antibodies are administered in combination with a second therapeutic agent. For example, the second therapeutic agent may comprise T cells, an immunomodulatory agent, a monoclonal antibody, a chemotherapeutic agent, hormone(s), drugs of any kind, surgery, radiation, etc. In non-limiting examples, the immunomodulatory agent is lenalidomide, the monoclonal antibody is rituximab, ofatumumab, or lumiliximab, and the chemotherapeutic agent is fludarabine or cyclophosphamide.

A composition of the present disclosure can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present disclosure, alone or in combination with other active agents, calculated in an amount sufficient to produce the desired effect, in association with a pharmaceutically acceptable diluent, carrier, or vehicle, where appropriate. The specifications for the unit dosage forms of the present disclosure depend on the particular pharmacodynamics associated with the pharmaceutical composition in the particular subject.

Desirably an effective amount or sufficient number of the isolated transduced NK cells is present in the composition and introduced into the subject such that long-term, specific, anti-tumor responses are established to reduce the size of a tumor or eliminate tumor growth or regrowth than would otherwise result in the absence of such treatment. Desirably, the amount of NK cells reintroduced into the subject causes a 10%, 20%, 30%, 40%, 50%, 60%, 70%, 80%, 90%, 95%, 98%, or 100% decrease in tumor size when compared to otherwise same conditions wherein the NK cells are not present.

Accordingly, the amount of compositions comprising NK cells and antibodies administered should take into account the route of administration and should be such that a sufficient number of the compositions comprising NK cells and antibodies will be introduced so as to achieve the desired therapeutic response. Furthermore, the amounts of each active agent included in the compositions described herein (e.g., the amount per each cell to be contacted or the amount per certain body weight) can vary in different applications. In general, the concentration of NK cells desirably should be sufficient to provide in the subject being treated at least from about 1×10⁶ to about 1× 10¹² NK cells, even more desirably, from about 1×10⁷ to about 5×10¹⁰ NK cells, although any suitable amount can be utilized either above, e.g., greater than 5×10⁸ cells, or below, e.g., less than 1×10⁷ cells. The dosing schedule can be based on well-established cell-based therapies (see, e.g., U.S. Pat. No. 4,690,915), or an alternate continuous infusion strategy can be employed.

These values provide general guidance of the range of compositions comprising NK cells and antibodies to be utilized by the practitioner upon optimizing the method of the present disclosure for practice of the present methods. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### III. Antibodies

Aspects of the disclosure relate to use of antibodies or functional fragments thereof in a composition also comprising certain NK cells. The term “antibody” refers to an intact immunoglobulin of any isotype, or a fragment thereof that can compete with the intact antibody for specific binding to the target antigen, such as through the antigen binding domain, and includes chimeric, humanized, fully human, monospecific, and multispecific (including at least bispecific and trispecific, and more) antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity. In specific embodiments, the antibody comprises an scFv.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4:302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)₂, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

Functional antibody fragments and antigen-binding fragments may be utilized. Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)₂ fragment means a bivalent antigen-binding fragment of a monoclonal antibody comprising two Fab′ fragments linked by a disulfide bridge at the hinge region. An F(ab′)₂ fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)₂ means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)₂ fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

In some cases, fragment crystallizable regions, Fc, are utilized. An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included. In some embodiments, antibodies that incorporate cytokines (including those referred to herein) may be utilized (such as with TRIKEs).

In some embodiments, an antibody is an antibody-drug conjugate. In some embodiments, an antibody may specifically be one or more of the following, although an antibody is not limited to these: [fam]-trastuzumab deruxtecan, Abciximab, Adalimumab, Ado-trastuzumab emtansine, Aducanumab, Alemtuzumab, Alirocumab, Amivantamab, Anifrolumab, Ansuvimab, Atezolizumab, Atoltivimab with Maftivimab and Odesivimab-ebgn (aka Inmazeb), Avelumab, Basiliximab, Belantamab mafodotin, Belimumab, Benralizumab, Bevacizumab, Bezlotoxumab, Bimekizumab, Blinatumomab, Brentuximab vedotin, Brodalumab, Brolucizumab, Burosumab, Canakinumab, Caplacizumab, Casirivimab+imdevimab, Catumaxomab, Cemiplimab, Certolizumab pegol, Cetuximab, Crizanlizumab, Daclizumab, Daratumumab, Denosumab, Dinutuximab, Donanemab, Dostarlimab, Dupilumab, Durvalumab, Eculizumab, Edrecolomab, Efalizumab, Elotuzumab, Emapalumab, Emicizumab, Enfortumab vedotin, Eptinezumab, Erenumab, Evinacumab, Evolocumab, Faricimab, Fremanezumab, Galcanezumab, Gemtuzumab, Golimumab, Guselkumab, Ibalizumab, Ibritumomab tiuxetan, Idarucizumab, Inebilizumab, Infliximab, Inolimomab, Inotuzumab, Ipilimumab, Isatuximab, Ixekizumab, Lanadelumab, Lecanemab, Loncastuximab tesirine, Margetuximab, Mepolizumab, Mirvetuximab soravtansine, Mogamulizumab, Mosunetuzumab, Moxetumomab pasudotox, Muromonab-CD3, Narsoplimab, Natalizumab, Naxitamab, Nebacumab, Necitumumab, Nirsevimab, Nivolumab, Obiltoxaximab, Obinutuzumab, Ocrelizumab, Ofatumumab, Olaratumab, Omalizumab, Omburtamab, Oportuzumab monatox, Palivizumab, Panitumumab, Pembrolizumab, Penpulimab, Pertuzumab, Polatuzumab vedotin, Ramucirumab, Ranibizumab, Ravulizumab, Raxibacumab, Regdanvimab, Relatlimab, Reslizumab, Retifanlimab, Risankizumab, Rituximab, Romosozumab, Sacituzumab govitecan, Sarilumab, Satralizumab, Secukinumab, Siltuximab, Sintilimab, Sotrovimab, Spesolimab, Sutimlimab, Tafasitamab, Tebentafusp, Teclistamab, Teplizumab, Teprotumumab, Tezepelumab, Tildrakizumab, Tislelizumab, Tisotumab vedotin, Tixagevimab, cilgavimab, Tocilizumab, Toripalimab, Tositumomab-I131, Tralokinumab, Trastuzumab, Tremelimumab, Ublituximab, Ustekinumab, Vedolizumab, or any combination thereof. In some embodiments, an antibody is Imgatuzumab. In some embodiments, an antibody is Margetuximab. In some embodiments, an antibody is Amivantamab. In some embodiments, an antibody is Obinutuzumab. In some embodiments, an antibody is IPH61 (also known as IPH6101 or SAR443579).

### IV. Pharmaceutical Compositions

Pharmaceutical compositions of the present disclosure comprise an effective amount of compositions comprising NK cells and antibodies dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises the compositions will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18^{th} Ed. Mack Printing Company, 1990, incorporated herein by reference).

The compositions comprising the NK cells and antibodies may be formulated into a composition in a free base, neutral or salt form. Pharmaceutically acceptable salts, where appropriate include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include compositions comprising the NK cells and antibodies and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a nanoparticle or in a lipid vehicle. For example, the compositions comprising the NK cells and antibodies may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The therapeutic compositions comprising the NK cells and antibodies of the disclosure may be administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice, in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

### A. Alimentary Compositions and Formulations

In particular embodiments of the present disclosure, the compositions comprising the NK cells and antibodies are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intravenously, intrathecally, intraventricularly, intra-tumorally, subcutaneously, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15^{th} Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other particular embodiments of the disclosure, the active compound compositions comprising the NK cells and antibodies may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### V. Combination Therapies

In certain embodiments, the compositions and methods of the present embodiments involve a cancer therapy that is additional to the compositions comprising the NK cells and antibodies. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor(s) or anti-metastatic agent(s). In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the compositions of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies (outside of the disclosed cell therapy) may be used in combination or in conjunction with methods of the embodiments.

In certain embodiments, additional immunotherapies comprise administration of one or more antibodies to a subject. In certain embodiments, the additional one or more antibodies that are administered to a subject may be the same one or more antibodies loaded onto an NK cell. In some embodiments, the additional one or more antibodies that are administered to a subject may target one or more of the same antigens as the one or more antibodies loaded onto an NK cell.

In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGF-beta R2.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Kits of the Disclosure

In some embodiments, a kit that can include, for example, NK cells, optionally one or more media and components for the production of NK cells, one or more antibodies or reagent(s) to produce the antibodies, and so forth are provided. In some embodiments, formulations may comprise a cocktail of factors, including in a form suitable for combining with NK cells. The reagent system or any kit component may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

In specific embodiments, the kit may comprise one or more cytokines, including at least IL-12, IL-15, IL-18, and/or IL-2, including in particular concentrations as described elsewhere herein. The kit may comprise any type of media, any component of a cryopreservation media, as described elsewhere herein. The kit may comprise cord blood (including pooled cord blood), antigen presenting cells of any kind, beads for depletion of particular NK cells (as described herein), vectors encoding one or more proteins as described herein, NK cells, antibodies or reagents to generate antibodies, etc.

Individual components may also be provided in a kit in concentrated amounts; in some embodiments, a component is provided individually in the same concentration as it would be in a solution with other components. Concentrations of components may be provided as 1x, 2x, 5x, 10x, or 20x or more.

### VII. Aspects

The following aspects describe certain inventions disclosed herein.
Aspect 1. A composition, comprising: (i) one or more engineered natural killer (NK) cells comprising one or more transgenes; and (ii) one or more one antibody molecules, wherein: (a) the antibody is monospecific, wherein an Fc region of the monospecific antibody binds the engineered NK cell and an antigen binding domain of the monospecific antibody is capable of binding a target antigen; or (b) the antibody is multispecific and one or more antigen binding domains of the antibody binds a target antigen and another antigen binding domain or domains binds another target antigen, or another antigen binding domain or domains of the antibody is capable of binding an NK cell surface antigen.
Aspect 2. The composition of aspect 1, wherein the engineered NK cell is or is not expanded.
Aspect 3. The composition of aspect 1 or 2, wherein the engineered NK cell is or is not pre-activated.
Aspect 4. The composition of any one of aspects 1-3, wherein the multispecific antibody is bispecific, trispecific, or multi-specific.
Aspect 5. The composition of any one of aspects 1-4, wherein in (a) the composition is further defined as a complex between the engineered NK cell and the monospecific antibody, through binding of the Fc region of the monospecific antibody to the engineered NK cell.
Aspect 6. The composition of any one of aspects 1-5, wherein the complex further comprises the antigen binding domain of the monospecific antibody bound to its target antigen.
Aspect 7. The composition of any one of aspects 1-4, wherein in (b) the composition is further defined as a complex between the engineered NK cell and the multispecific antibody, through binding of the antigen binding domain or domains of the multispecific antibody that binds the surface antigen or antigens on the engineered NK cell.
Aspect 8. The composition of any one of aspects 1˜4 and 7, wherein the one or more antigen binding domain or domains of the antibody is bound to its target antigen.
Aspect 9. The composition of any one of aspects 1-6, wherein the target antigen is a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, Epidermal Growth Factor receptor (EGFR), epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.
Aspect 10. The composition of any one of aspects 1-9, wherein the engineered NK cell surface antigen is CD16, CD32, CS1, CD56, CD64, NKG2D, NKG2C, DNAM, 2B4, CD2, an NCR, NKp30, NKp44, NKp46, or KIR.
Aspect 11. The composition of any one of aspects 1-9, wherein the NK cells are sourced from cord blood, and wherein the source of the cord blood is cord blood from 1 donor or pooled from 2 or more individual cord blood units.
Aspect 12. The composition of aspect 11, wherein the CB is pooled from 3, 4, 5, 6, 7, or 8 individual cord blood units.
Aspect 13. The composition of any one of aspects 1-12, wherein the engineered NK cells are derived from cord blood mononuclear cells, from cord blood hematopoietic stem cells, from iPSCs, from peripheral blood NK cells, or from NK cell lines such as NK-92.
Aspect 14. The composition of any one of aspects 1-13, wherein the engineered NK cells are CD56+, CD3−, or both.
Aspect 15. The composition of any one of aspects 1-14, wherein said composition is used fresh or was cryopreserved.
Aspect 16. The composition of any one of aspects 1-15, wherein a source of the engineered NK cells is a fresh source or cryopreserved repository.
Aspect 17. The composition of aspect 15 or 16, wherein when the engineered NK cells are sourced from cryopreservation, the engineered NK cells were cryopreserved in a medium comprising at least one cryoprotectant, at least one serum or non-serum alternative to serum, and optionally at least one cytokine and/or at least one growth factor.
Aspect 18. The composition of aspect 17, wherein the cryoprotectant is dimethyl sulfoxide (DMSO), glycerin, glycerol, hydroxyethyl starch, dextran trehalose, or a combination thereof.
Aspect 19. The composition of aspect 17 or 18, wherein the non-serum alternative comprises platelet lysate and/or a blood product lysate or human or animal serum albumin.
Aspect 20. The composition of any one of aspects 17-19, wherein the at least one cytokine is a natural protein, a recombinant protein, a synthetic protein, or a mixture thereof.
Aspect 21. The composition of any one of aspects 17-20, wherein the at least one cytokine is interleukin (IL)-1, IL-2, IL-3, IL-4, IL-6, IL-7, IL-9, IL-10, IL-12, IL-13, IL-15, IL-17, IL-18, IL-21, IL-22, IL-23, interferon, tumor necrosis factor, stem cell factor, FLT3-ligand, APRIL, thrombopoietin, erythropoietin, or a combination thereof.
Aspect 22. The composition of any one of aspects 1-21, wherein the engineered NK cells comprise one or more transgenes encoding one or more engineered receptors.
Aspect 23. The composition of aspect 22, wherein the one or more engineered receptor is a chimeric antigen receptor, a T cell receptor, receptors recognizing the FC portion of Ig (e.g., CD16, CD32, CD64, etc.) chemokine receptor, homing receptor, cytokine gene, chimeric cytokine receptor, or any combination thereof.
Aspect 24. The composition of any one of aspects 1-23, wherein the engineered NK cells express one or more transgenes encoding one or more heterologous cytokines and/or cytokine receptors.
Aspect 25. The composition of aspect 24, wherein the one or more heterologous cytokine is IL-2, IL-4, IL-7, IL-12, IL-15, IL-18, IL-21 and/or IL-23.
Aspect 26. The composition of any one of aspects 1-25, wherein the engineered NK cells express one or more transgenes encoding one or more receptors to enhance their binding to an antibody.
Aspect 27. The composition of aspect 26, wherein the one or more receptor comprises or is an Fc receptor.
Aspect 28. The composition of aspect 26, wherein the receptor is CD16, CD32, CD64, or a combination thereof.
Aspect 29. The composition of any one of aspects 1-28, wherein the engineered NK cells express one or more transgenes encoding one or more suicide genes.
Aspect 30. The composition of any one of aspects 1-29, wherein the composition is comprised in a solution or solid comprising one or more cryoprotectants.
Aspect 31. The composition of any one of aspects 1-30, wherein the composition is comprised in a pharmaceutically acceptable carrier.
Aspect 32. A method of producing the composition of any one of aspects 1-31, comprising the steps of: (i) optionally expanding engineered NK cells in a culture comprising an effective amount of: (a) a cytokine selected from the group consisting of IL-2, IL-15, IL-18, IL-21 and a combination thereof; and (b) antigen presenting cells/feeders, fragments of antigen presenting cells/feeders, or NK cell-activating beads; and (ii) providing the antibody molecules to the engineered NK cells, and when expanding providing the antibody molecules to the engineered NK cells before and/or after expanding.
Aspect 33. The method of aspect 32, wherein the method comprises a pre-activating step prior to and/or after the expanding step, wherein the engineered NK cells are pre-activated in a culture comprising an effective concentration of one or more of IL-2, IL-12, IL-15, and IL-18.
Aspect 34. The method of aspect 33, wherein the culture comprises an effective concentration of two or more of IL-2, IL-12, IL-15, and IL-18.
Aspect 35. The method of aspect 33 or 34, wherein the culture comprises an effective concentration of three or more of IL-2, IL-12, IL-15, and IL-18.
Aspect 36. The method of any one of aspects 32-35, wherein the culture comprises an effective concentration of IL-12, IL-15, and IL-18.
Aspect 37. The method of any one of aspects 32-36, wherein IL-12 is utilized in lieu of IL-15 in the culture.
Aspect 38. The method of any one of aspects 32-37, wherein the providing step is further defined as culturing the engineered NK cells with the antibody molecules for a specific duration of time or combining the engineered NK cells and the antibody molecules just prior to infusion.
Aspect 39. The method of aspect 38, wherein the duration of time is about 5 minutes to about 24 hours or more.
Aspect 40. The method of aspect 38 or 39, wherein the culture comprises Plasma-Lyte A and/or human serum albumin.
Aspect 41. The method of any one of aspects 32-40, wherein following culture, the compositions are infused into a recipient subject without washing first.
Aspect 42. The method of any one of aspects 32-40, wherein following culture, the compositions are infused into a recipient subject following one or more washes.
Aspect 43. The method of any one of aspects 32-42, wherein the engineered NK cells are depleted of CD3+, CD14+ and/or CD19+ cells.
Aspect 44. The method of aspect 43, wherein the depleting step occurs prior to the pre-activation step, and/or prior to expansion with feeder cells and/or NK cell-activating beads, and/or prior to culture with one or more cytokines, and/or prior to infusion.
Aspect 45. The method of any one of aspects 32-44, further comprising the step of obtaining the NK cells from cord blood, wherein the cord blood does not comprise cord tissue.
Aspect 46. The method of any one of aspects 32-45, wherein the antigen presenting cells are artificial (aAPCs).
Aspect 47. The method of aspect 46, wherein the aAPCs express CD137 ligand.
Aspect 48. The method of aspect 46 or 47, wherein the aAPCs further express a membrane-bound cytokine.
Aspect 49. The method of aspect 48, wherein the membrane-bound cytokine is membrane-bound IL-21 (mIL-21) or membrane-bound IL-15 (mIL-15).
Aspect 50. The method of any one of aspects 46-49, wherein the aAPCs have essentially no expression of endogenous HLA class I, II, or CD1d molecules.
Aspect 51. The method of any one of aspects 46-50, wherein the aAPCs express ICAM-1 (CD54) and/or LFA-3 (CD58) or CD48.
Aspect 52. The method of any one of aspects 46-51, wherein the aAPCs are further defined as leukemia cell-derived aAPCs.
Aspect 53. The method of aspect 52, wherein the leukemia-cell derived aAPCs are K562 cells engineered to express CD137 ligand and/or mIL-21.
Aspect 54. The method of aspect 53, wherein the K562 cells are engineered to express CD137 ligand and mIL-21.
Aspect 55. The method of any one of aspects 46-54, wherein the aAPCs have been engineered by retroviral transduction.
Aspect 56. The method of any one of aspects 46-55, wherein the aAPCs are irradiated. Aspect 57. The method of any one of aspects 32-56, wherein the pre-activating step is

for 10-20 hours.
Aspect 58. The method of any one of aspects 32-57, wherein the pre-activating step is for 14-18 hours.
Aspect 59. The method of any one of aspects 32-58, wherein the pre-activating step is for 16 hours.
Aspect 60. The method of any one of aspects 32-59, wherein the culture for the pre-activating step comprises IL-18 and/or IL-15 at a concentration of 1-1000 ng/ml.
Aspect 61. The method of any one of aspects 32-60, wherein the culture for the pre-activating step comprises IL-18 and/or IL-15 at a concentration of 1-1000 ng/ml.
Aspect 62. The method of any one of aspects 32-61, wherein the culture for the pre-activating step comprises IL-18 and/or Il-15 at a concentration of 1-1000 ng/mL.
Aspect 63. The method of any one of aspects 32-62, wherein the culture for the pre-activating step comprises IL-12 at a concentration of 0.1-1000 ng/ml.
Aspect 64. The method of any one of aspects 32-63, wherein the culture for the pre-activating step comprises IL-12 at a concentration of 1-1000 ng/ml.
Aspect 65. The method of any one of aspects 32-64, wherein the culture for the pre-activating step comprises IL-12 at a concentration of 10 ng/ml.
Aspect 66. The method of any one of aspects 32-65, further comprising washing the pre-activated engineered NK cells prior to and/or after the expanding step.
Aspect 67. The method of any one of aspects 32-66, wherein engineered NK cells are activated at least twice or more during the expansion step with IL-12, IL-15, IL-18, IL-2 or any combination thereof.
Aspect 68. The method of aspect 67, wherein washing is performed multiple times.
Aspect 69. The method of any one of aspects 34-68, wherein expanding is for 5-60 days.
Aspect 70. The method of any one of aspects 32-69, wherein the expanding is for 12-16 days.
Aspect 71. The method of any one of aspects 32-69, wherein the expanding is for 18-24 days.
Aspect 72. The method of any one of aspects 32-71, wherein the pre-activated engineered NK cells and aAPCs are present in the expansion culture at a ratio of 3:1 to 1:3.
Aspect 73. The method of aspect 72, wherein the pre-activated engineered NK cells and aAPCs are present in the expansion culture at a ratio 1:2.
Aspect 74. The method of any one of aspects 32-73, wherein the expansion culture further comprises IL-2.
Aspect 75. The method of aspect 74, wherein the IL-2 is present at a concentration of 10-500 U/mL.
Aspect 76. The method of aspect 75, wherein the IL-2 is present at a concentration of 100-300 U/mL.
Aspect 77. The method of aspect 76, wherein the IL-2 is present at a concentration of 200 U/mL.
Aspect 78. The method of any one of aspects 32-77, wherein the IL-12, IL-18, IL-15, and/or IL-2 are recombinant.
Aspect 79. The method of any one of aspects 32-78, wherein the IL-2 is replenished in the expansion culture every 2-3 days.
Aspect 80. The method of any one of aspects 32-79, wherein the APCs are added to the expansion culture at least a second time.
Aspect 81. The method of any one of aspects 32-80, wherein one or more steps of the method are performed in serum-free media.
Aspect 82. A method of treating a disease or disorder in a subject, comprising administering a therapeutically effective amount of the compositions of any one of aspects 1-31 to the subject.
Aspect 83. The method of aspect 82, wherein the disease or disorder is cancer, inflammation, graft versus host disease, transplant rejection, an autoimmune disorder, an immunodeficiency disease, a B cell malignancy, or an infection.
Aspect 84. The method of aspect 82 or 83, wherein the cancer is a hematological cancer or a solid tumor.
Aspect 85. The method of aspect 84, wherein the hematological cancer is a leukemia selected from the group consisting of an acute lymphoblastic leukemia (ALL), chronic lymphocytic leukemia (CLL), multiple myeloma, acute myelogenous leukemia (AML), and a chronic myelogenous leukemia (CML).
Aspect 86. The method of any one of aspects 82-85, wherein the engineered NK cells are allogeneic with respect to the subject.
Aspect 87. The method of any one of aspects 82-86, wherein the engineered NK cells are autologous with respect to the subject.
Aspect 88. The method of aspect 82, wherein the disorder is graft versus host disease (GVHD).
Aspect 89. The method of aspect 82, wherein the disorder is multiple sclerosis, inflammatory bowel disease, rheumatoid arthritis, type I diabetes, systemic lupus erythematosus, contact hypersensitivity, asthma or Sjogren's syndrome.
Aspect 90. The method of any one of aspects 82-89, wherein the subject is a human.
Aspect 91. The method of any one of aspects 82-90, further comprising administering at least a second therapeutic agent to the subject.
Aspect 92. The method of aspect 91, wherein the at least a second therapeutic agent is a therapeutically effective amount of one or more anti-cancer agents, one or more immunomodulatory agents, and/or one or more immunosuppressive agents.
Aspect 93. The method of aspect 92, wherein the anti-cancer agent is chemotherapy, radiotherapy, gene therapy, surgery, hormonal therapy, anti-angiogenic therapy or immunotherapy.
Aspect 94. The method of aspect 92, wherein the immunosuppressive agent is a calcineurin inhibitor, an mTOR inhibitor, an antibody, a chemotherapeutic agent irradiation, a chemokine, an interleukins or an inhibitor of a chemokine or an interleukin.
Aspect 95. The method of any one of aspects 91-94, wherein the composition and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.
Aspect 96. The method of any one of aspects 91-95, wherein the second therapeutic agent is an antibody.
Aspect 97. The method of aspect 96, wherein the antibody if a monoclonal, bispecific, or multi-specific antibody.
Aspect 98. A method of treating a subject with cancer with an engineered NK cell, the method comprising: a) optionally stimulating a population of engineered NK cells by pre-activation with a cytokine cocktail; b) optionally expanding the engineered NK cells; and ci) loading the engineered NK cells ex vivo with an antibody and then administering the loaded engineered NK cells to the subject, or cii) loading the engineered NK cells in vivo with antibody by administering the engineered NK cells within 1 hour of administration of an antibody; wherein the engineered NK cell comprises one or more transgenes, and wherein the loading comprises exposure of engineered NK cells to the antibody for an amount of time effective for binding of the antibody to the engineered NK cell surface.
Aspect 99. The method of aspect 98, wherein loading the engineered NK cells comprises exposure of the engineered NK cells to the antibody for greater than or equal to about 1 hour, or less than or equal to about 1 hour.
Aspect 100. The method of aspect 98 or 99, wherein a plurality of the engineered NK cells remain loaded with the antibody for equal to or greater than 5 days.
Aspect 101. The method of any one of aspects 98-100, wherein the antibody is bound to the engineered NK cell through interaction with a CD16 surface protein.
Aspect 102. The method of any one of aspects 98-101, wherein the engineered NK cells are derived from cord blood.
Aspect 103. The method of any one of aspects 98-102, wherein the engineered NK cells are activated ex-vivo.
Aspect 104. The method of any one of aspects 98-103, wherein the engineered NK cells are pre-activated with IL-18, IL-15, and IL-12.
Aspect 105. The method of any one of aspects 98-104, wherein the antibody is an anti-EGFR antibody.
Aspect 106. The method of any one of aspects 98-104, wherein the antibody is an anti-EGFR2 antibody.
Aspect 107. The method of any one of aspects 98-104, wherein the antibody is a monospecific antibody.
Aspect 108. The method of aspect 105, wherein the antibody is Imgatuzumab.
Aspect 109. The method of aspect 106, wherein the antibody is Margetuximab.
Aspect 110. The method of any one of aspects 98-105, wherein the antibody is a bispecific antibody.
Aspect 111. The method of aspect 110, wherein the antibody is Amivantamab.
Aspect 112. The method of any one of aspects 98-104, wherein the antibody is an anti-CD123 antibody.
Aspect 113. The method of any one of aspects 98-104, wherein the antibody is a bi-specific antibody.
Aspect 114. The method of any one of aspects 98-104, wherein the antibody comprises an antigen binding domain that targets CD123, and an antigen binding domain that targets NKp46.
Aspect 115. The method of aspect 105, wherein the antibody is IPH61.
Aspect 116. The method of any one of aspects 98-115, wherein the one or more transgenes comprise an engineered receptor.
Aspect 117. The method of aspect 116, wherein the engineered receptor comprises a chimeric antigen receptor (CAR), a T cell receptor, chemokine receptor, chimeric cytokine receptor, or any combination thereof.
Aspect 118. The method of aspect 116 or 117, wherein the engineered receptor comprises a CAR.
Aspect 119. The method of any one of aspects 98-118, wherein the one or more transgenes comprise a cytokine.
Aspect 120. The method of aspect 119, wherein the cytokine is IL-2, IL-12, IL-15, IL-18, and/or IL-21.
Aspect 121. The method of any one of aspects 98-120, wherein the engineered NK cells comprise an engineered mutation in an endogenous gene.
Aspect 122. The method of aspect 121, wherein the engineered mutation in an endogenous gene is in a CISH, CD3δ, Glucocorticoid Receptor, and/or TGFBR2 gene.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1-Preparation and Use of Compositions

Compositions comprising particular engineered NK cells and also antibodies that are able to bind the engineered NK cells are prepared and, in particular cases, are given in an effective amount to an individual in need of treatment. In particular embodiments, engineered NK cells that are autologous or allogeneic with respect to the individual are administered in an effective amount to the individual, and the engineered NK cells are particularly cord blood cells. The engineered NK cells may or may not be sourced from a cryopreservation repository and in some cases were cryopreserved in a particular cryopreservation medium. The engineered NK cells are optionally pre-activated and optionally expanded prior to exposing an effective amount of the engineered NK cells to the antibodies under conditions that allow the antibodies to bind the engineered NK cells through the appropriate antigen binding fragment and/or Fc domain. Following this, an effective amount of the compositions are provided to the individual in need of them.

### Example 2-Monoclonal Antibodies and Nk Cells

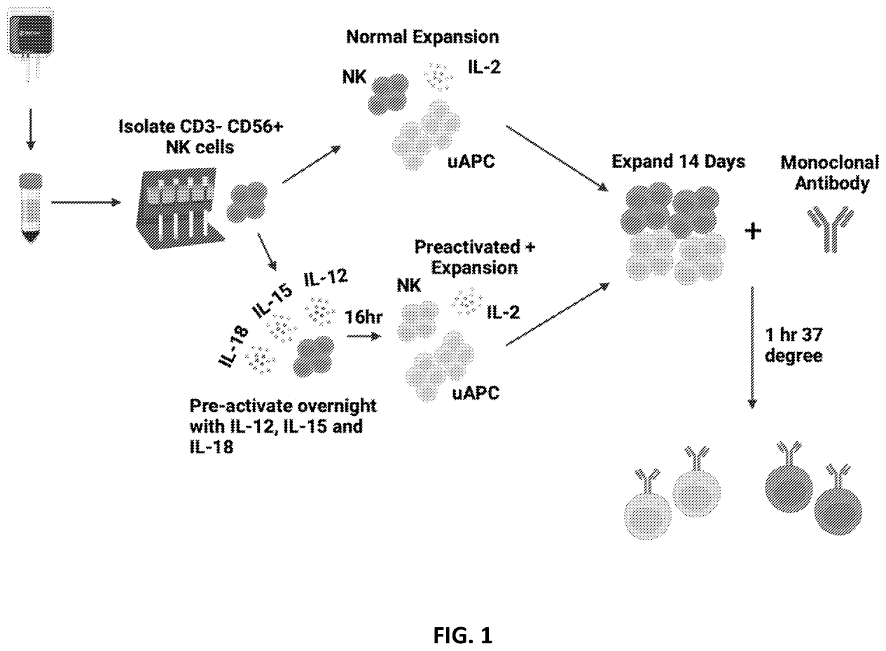

The present example encompasses preparation and use of NK cells preloaded with antibodies. FIG. 1 shows one example of an experimental procedure for NK cell expansion and antibody loading. NK cells may be obtained from blood that has been processed, and in specific embodiments certain NK cells are isolated, such as those that are CD3−CD56+, in specific embodiments. Optionally the NK cells are expanded, for example in the presence of IL-2 and universal antigen presenting cells or other feeder cells, and the expansion step may be of any suitable duration, such as up to about 14 days, in specific cases. In another optional embodiment, the NK cells are pre-activated prior to expansion. Although any pre-activation step may be utilized, in specific embodiments the pre-activation step encompasses exposure to one or more cytokines, such as one or more of IL-12, IL-15, and/or IL-18. The pre-activation step may take any suitable length of duration, but in specific embodiments it is up to about 16 hours. Following this step, the NK cells optionally may be expanded, including in the presence of universal antigen presenting cells and IL-2, for example. The cultured cell may be activated again with IL2, IL-18 or IL-15 or any combination for these cytokines during expansion. Following expansion, the NK cells are exposed to a suitable amount of antibody under sufficient conditions such that the antibody becomes bound to the outside of the NK cells though a particular antigen to which the antibody is directed. In specific cases, the antibody is a monoclonal antibody. The conditions for loading of the antibodies onto the NK cells may be under particular temperatures for a specific duration, such as one hour at 37° C.

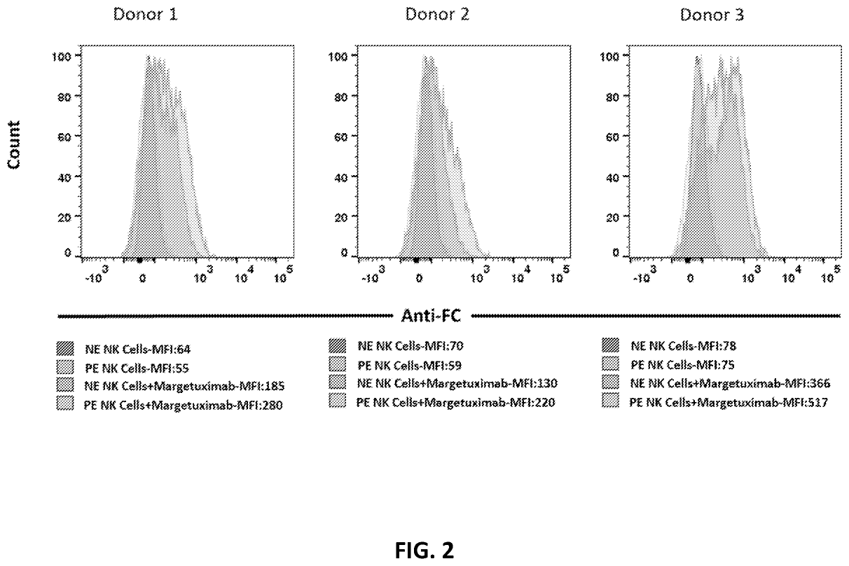

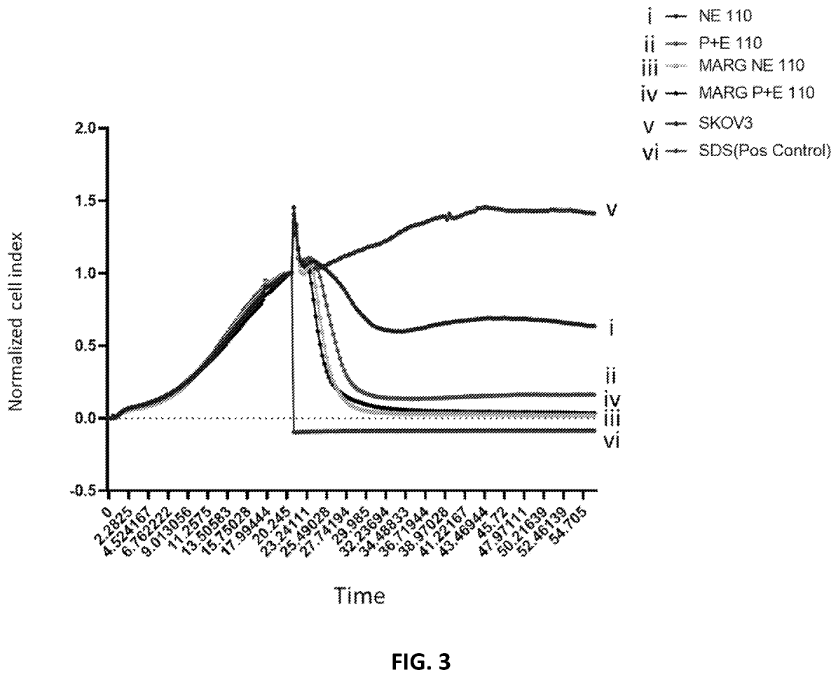

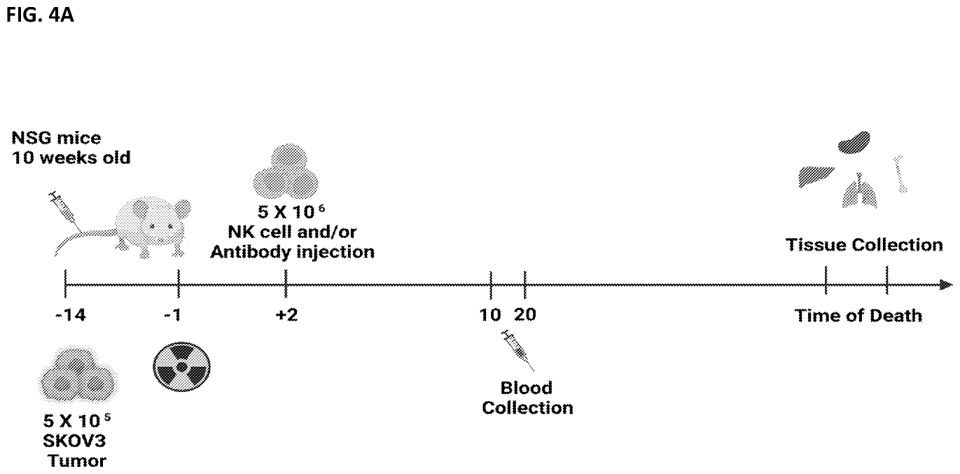

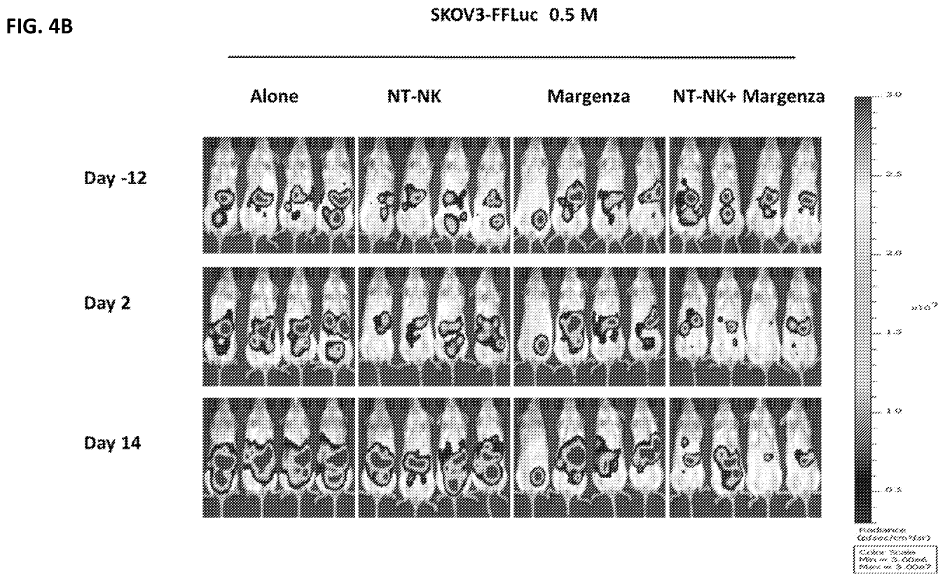

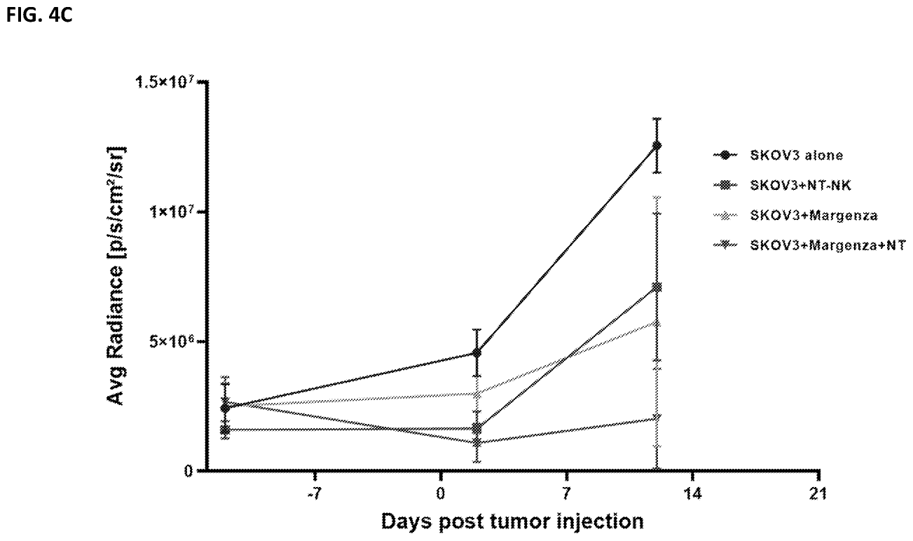

FIG. 2 provides validation of Margetuximab (anti-HER2 monoclonal antibody) binding to NK cells. NK cells were derived from cord blood and were either normally expanded (NE) with irradiated (100 Gy) uAPC feeder cells (2:1 feeder cell:NK ratio) and recombinant human IL-2 (200 U/ml) in 50:50 Click's/RPMI media or pre-activated with IL-12 (10 ng/ml), IL-15 (50 ng/ml), and IL-18 (50 ng/ml) for 16 hrs and then washed and expanded (PE) with irradiated uAPC and IL-2 (200 U/ml). NK cells were loaded with Margetuximab 1 μg/ml for one hour at 37° C. in Click's/RPMI media and were washed before validating the binding of Margetuximab to NK cells with flow cytometry. Non-loaded NE and PE NK cells were used as negative control. Margetuximab binding was detected by flow cytometry staining using Alexa-Fluor 647 affinity-purified F(ab′)₂ fragment goat anti-human IgG (H+L) antibody. The graphs show that PENK cells show higher level of Margetuximab binding compared to NE NK cells. Histograms provide data from 3 different cord blood NK cell donors. Margetuximab-loaded NK cells show enhanced cytotoxicity against HER2+ tumor cells (FIG. 3). Provided therein is an Xcelligence cytotoxicity assay showing normalized cell index of HER2+SKOV3 (ovarian cancer) cultured alone, or with NK cells at 2:1 effector to target (E:T) ratio. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Margetuximab 1 μg/ml for one hour at 37° C. in Click's/RPMI media and then washed before the assay. Data indicate that Margetuximab loading enhances the cytotoxicity of NE and PE NK cells against tumor cells. Margetuximab-loaded NK cells led to enhanced tumor control in an NSG mouse model of HER2+ovarian cancer (SKOV3) (FIG. 4). In FIG. 4A, there is one example of a schema of experimental plan for SKOV3 mouse model. In FIG. 4B, bioluminescent imaging shows tumor growth over time in mice engrafted with SKOV3 transduced with Fireflyluciferase (FFluc) and either left untreated or treated with NK cells alone, Margetuximab alone, or NK cells pre-loaded with Margetuximab. FIG. 4C provides a graph showing average radiance over time of the four groups of mice described in panel 4A.

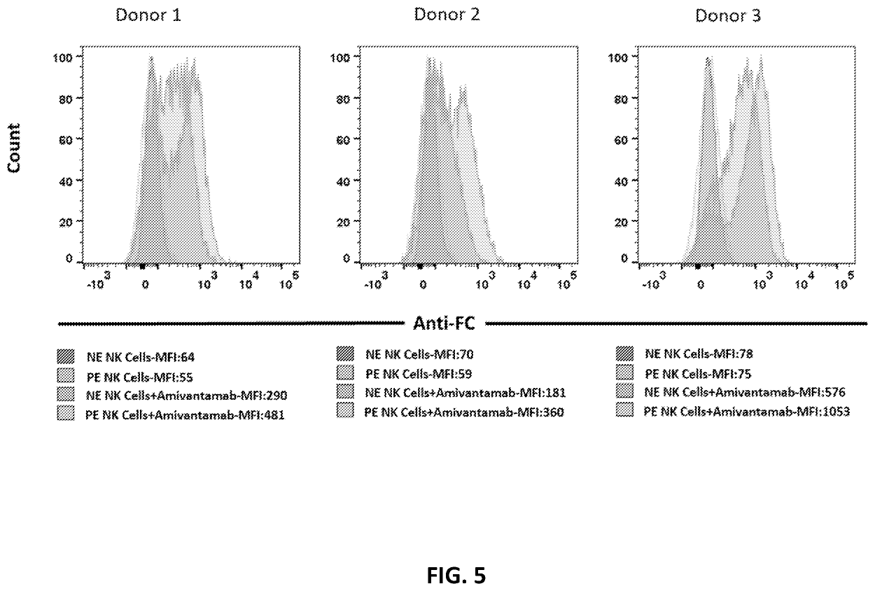

In FIG. 5, validation of Amivantamab (EGFR-MET bispecific antibody) binding to the NK cells is demonstrated. NK cells were derived from cord blood and were either normally expanded (NE) with irradiated (100 Gy) uAPC feeder cells (2:1 feeder cell:NK ratio) and recombinant human IL-2 (200 U/ml) in 50:50 Click's/RPMI media or pre-activated with IL-12 (10 ng/ml), IL-15 (50 ng/ml), and IL-18 (50 ng/ml) for 16 hrs and then washed and expanded (PE) with irradiated uAPC and IL-2 (200 U/ml). NK cells were loaded with Amivantamab 1 μg/ml for one hour at 37° C. in Click's/RPMI media and were washed before validating the binding of Amivantamab to NK cells with flow cytometry. Non-loaded NE and PE NK cells were used as negative control. Amivantamab binding was detected by flow cytometry staining using Alexa-Fluor647 affinity-purified F(ab′)₂ fragment goat anti-human IgG (H+L) antibody. The graphs show that PE NK cells show higher level of Amivantamab binding compared to NE NK cells. Histograms show data from 3 different cord blood NK cell donors.

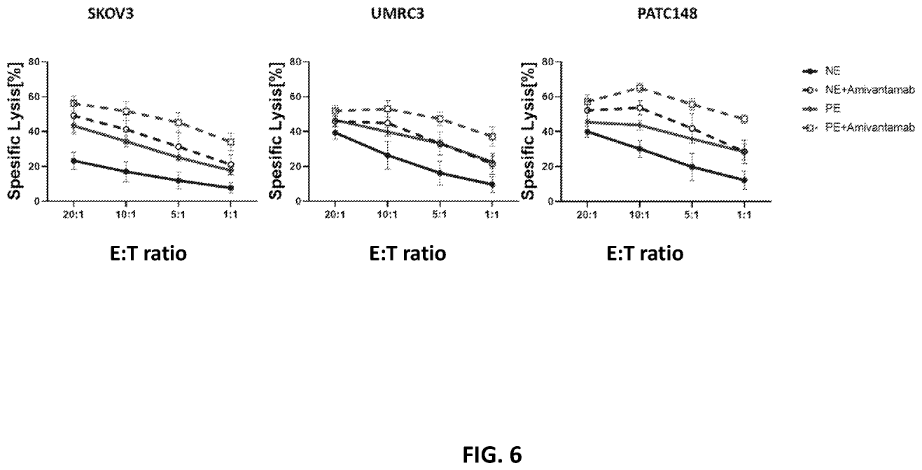

Amivantamab-loaded NK cells show enhanced cytotoxicity against EGFR+/cMET+tumor cells (FIG. 6). Chromium release assay showing specific lysis of 3 EGFR+/c-MET+ cancer cells lines SKOV3 (ovarian cancer), UMRC3 (renal cell carcinoma), PATC-148 (pancreatic ductal adenocarcinoma) using 3 different cord blood NK cells (N=3 per assay) at various effector to target (E:T) ratios (20:1, 10:1, 5:1, 1:1) is provided. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Amivantamab 1 μg/ml for one hour at 37° C. in Click's/RPMI media and then washed before the assay. Amivantamab loading enhances the cytotoxicity of NE and PE NK cells against tumor cells.

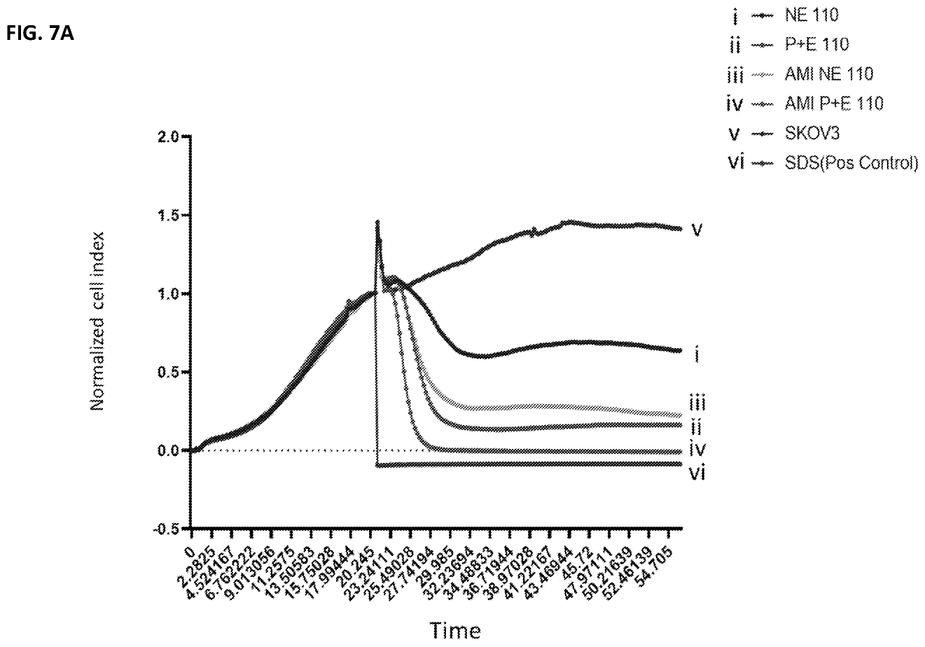

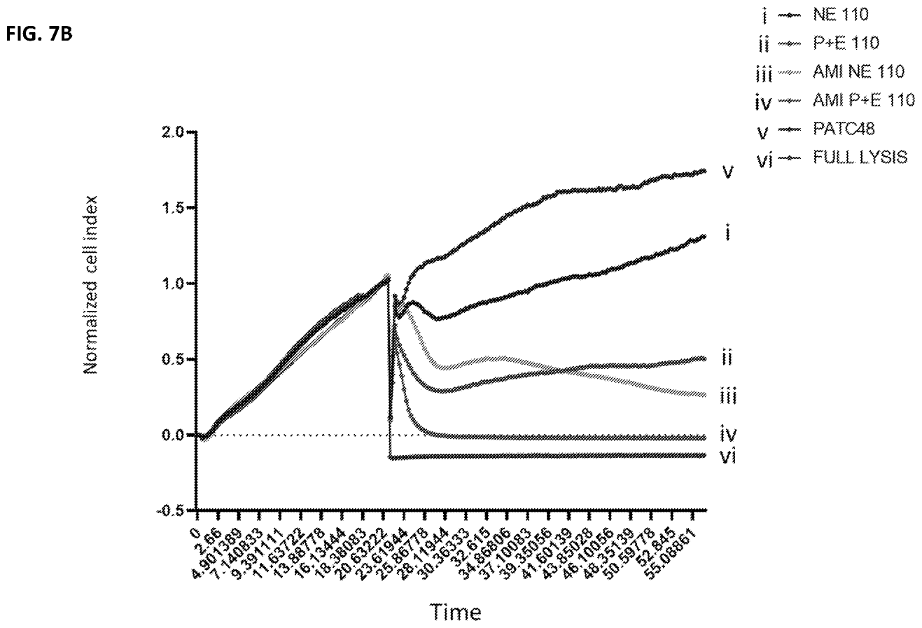

In FIGS. 7A and 7B, Amivantamab-loaded NK cells show enhanced cytotoxicity against EGFR+/cMET+ tumor cells. Xcelligence cytotoxicity assay showing normalized cell index of 2 EGFR+/c-MET+ cancer cells lines SKOV3 (ovarian cancer; FIG. 7A) and PATC-148 (pancreatic ductal adenocarcinoma; FIG. 7B) using 3 different cord blood NK cells (N=3 per assay) at 2:1 effector to target (E:T) ratio. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Amivantamab 1 μg/ml for one hour at 37° C. in Click's/RPMI media and then washed before the assay. Amivantamab loading enhances the cytotoxicity of NE and PE NK cells against tumor cells.

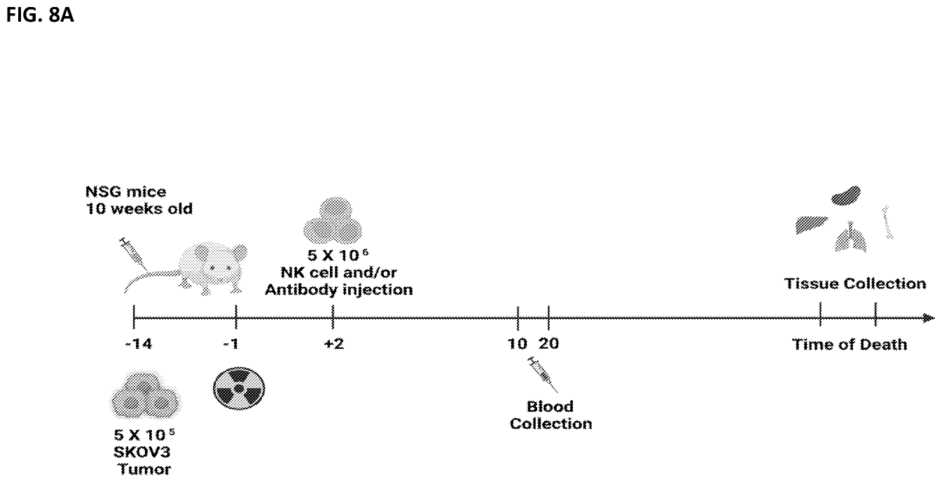

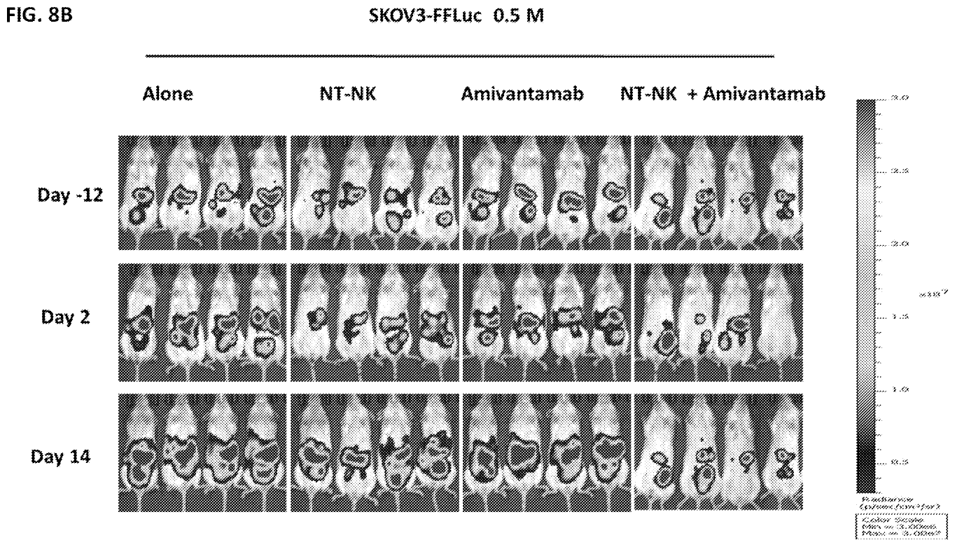

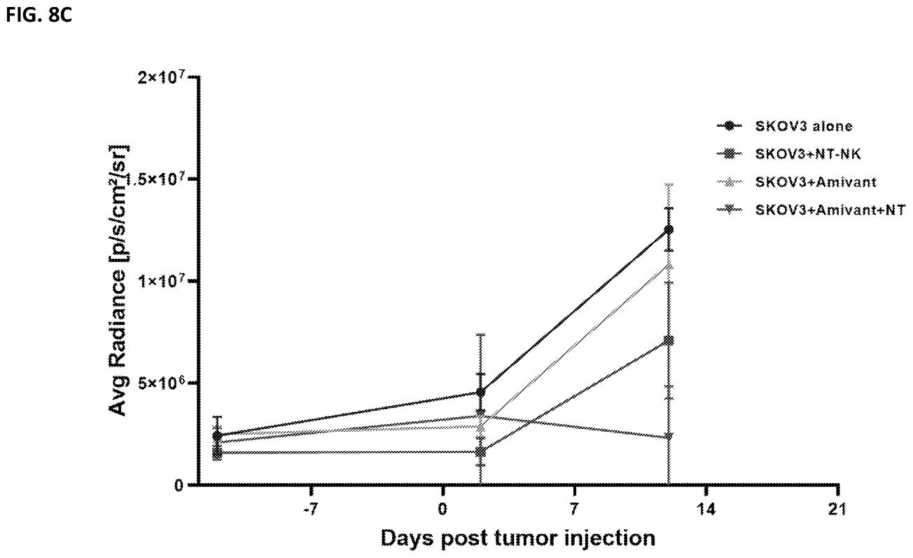

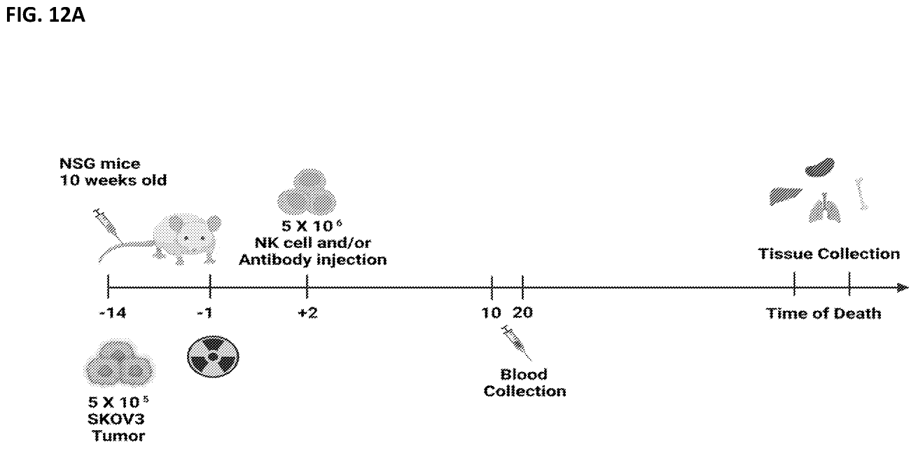

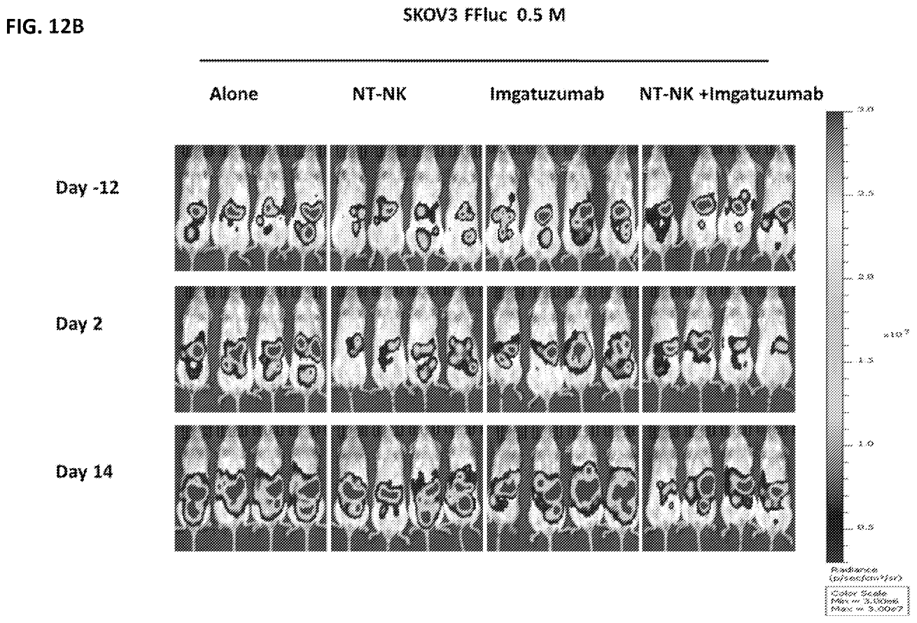

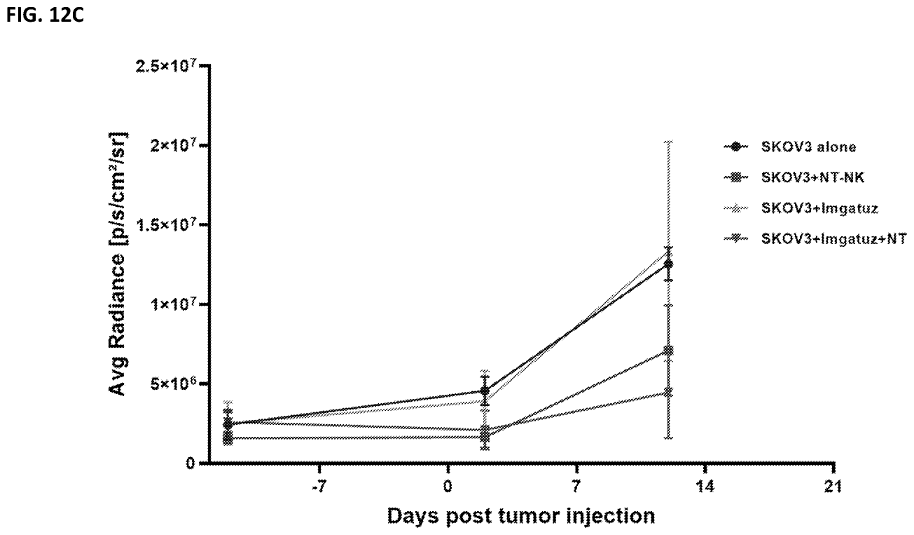

Amivantamab-loaded NK cells led to enhanced tumor control in an NSG mouse model of EGFR+/c-MET+ovarian cancer (SKOV3) (FIG. 8). An example of a schema of experimental plan for SKOV3 mouse model is provided in FIG. 12A. In FIG. 8B, bioluminescent imaging shows tumor growth over time in mice engrafted with SKOV3 transduced with Fireflyluciferase (FFluc) and either left untreated or treated with NK cells alone, Amivantamab alone, or NK cells pre-loaded with Amivantamab. FIG. 8C provides a graph showing average radiance over time of the four groups of mice described in FIG. 8A.

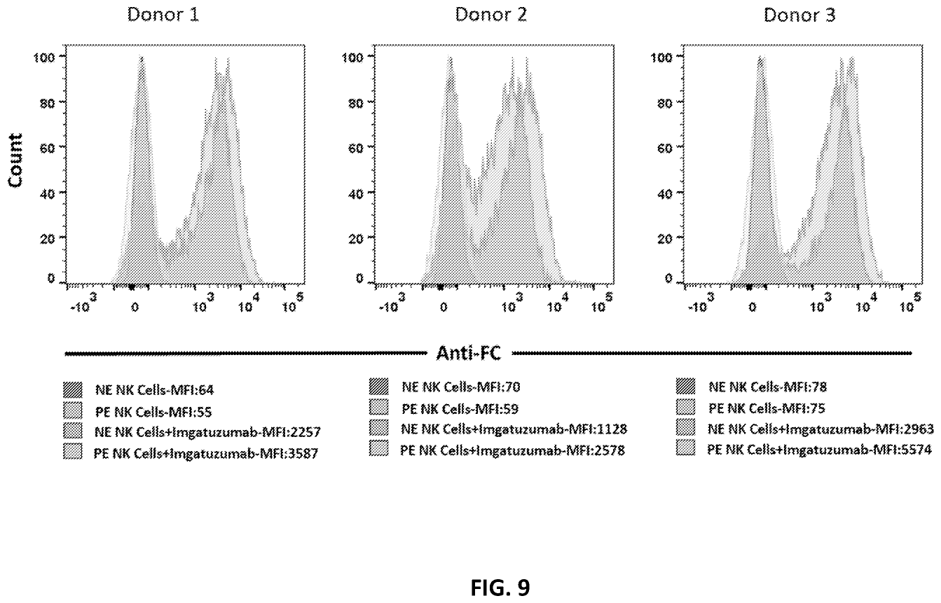

NK cells were loaded with one example of an antibody (FIG. 9), Imgatuzumab (an anti-EGFR monoclonal antibody). NK cells were derived from cord blood and were either normally expanded (NE) with irradiated (100 Gy) uAPC feeder cells (2:1 feeder cell:NK ratio) and recombinant human IL-2 (200 U/ml) in 50:50 Click's/RPMI media or pre-activated with IL-12 (10 ng/ml), IL-15 (50 ng/ml), and IL-18 (50 ng/ml) for 16 hrs and then washed and expanded (PE) with irradiated uAPC and IL-2 (200 U/ml). NK cells were loaded with Imgatuzumab at 1 μg/ml for one hour at 37° C. in Click's/RPMI media and were washed before validating the binding of Imgatuzumab to NK cells with flow cytometry. Non-loaded NE and PE NK cells were used as negative control. Imgatuzumab binding was detected by flow cytometry staining using Alexa-Fluor647 affinity-purified F(ab′)₂ fragment goat anti-human IgG (H+L) antibody. The graphs show that PE NK cells show higher level of Imgatuzumab binding compared to NE NK cells. Histograms show data from 3 different cord blood NK cell donors.

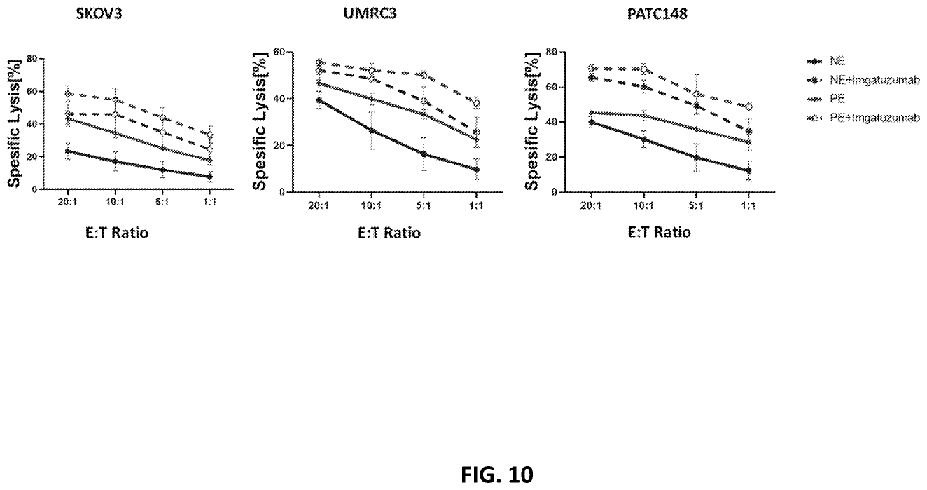

Imgatuzumab-loaded NK cells show enhanced cytotoxicity against tumor cells (FIG. 10). Chromium release assay showing specific lysis of 3 EGFR+ cancer cells lines (SKOV3 (ovarian cancer), UMRC3 (renal cell carcinoma), PATC-148 (pancreatic ductal adenocarcinoma)) using 3 different cord blood NK cells (N=3 per assay) at various effector to target (E:T) ratios (20:1, 10:1, 5:1, 1:1). NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Imgatuzumab 1 μg/ml for one hour at 37° C. in Click's/RPMI media and then washed before the assay. Data show that Imgatuzumab loading enhances the cytotoxicity of NE and PE NK cells against tumor cells.

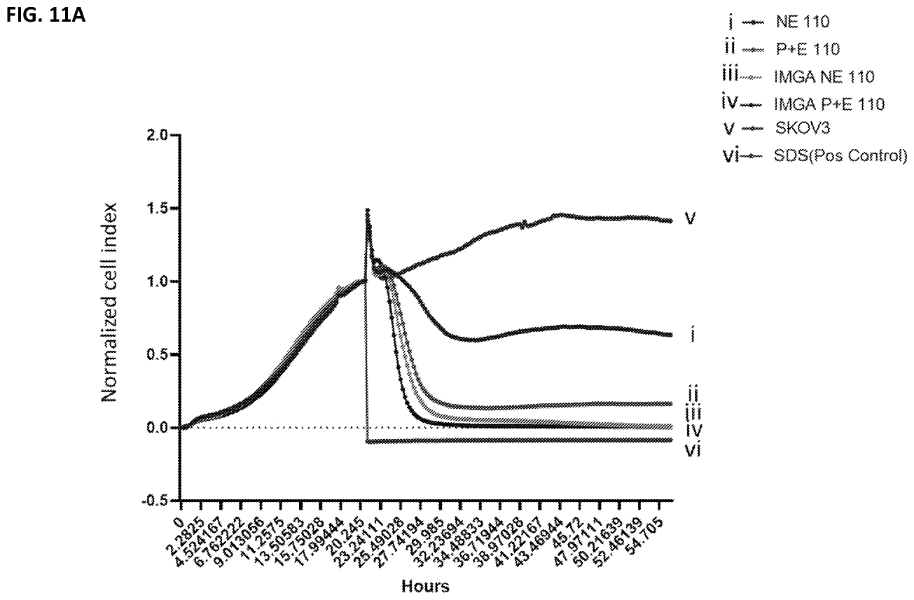

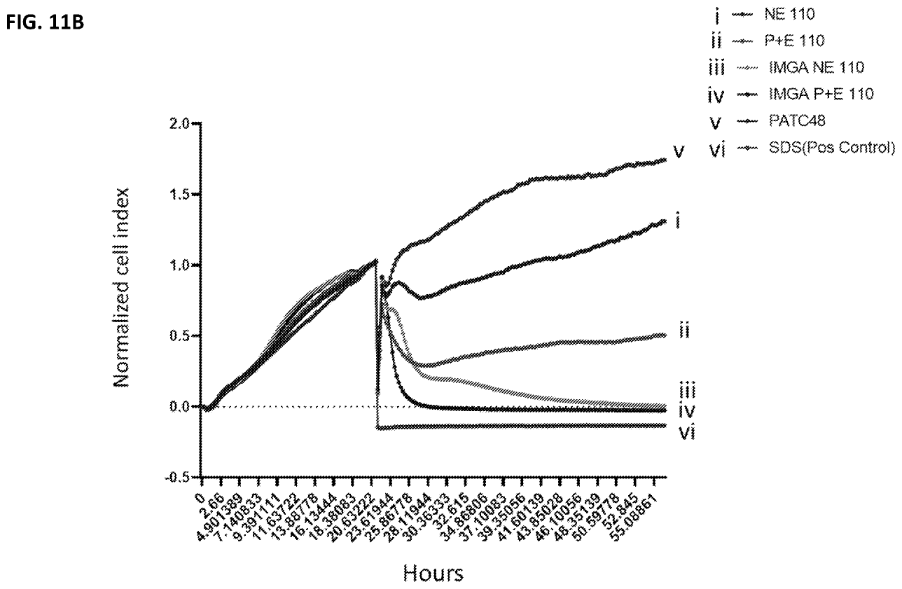

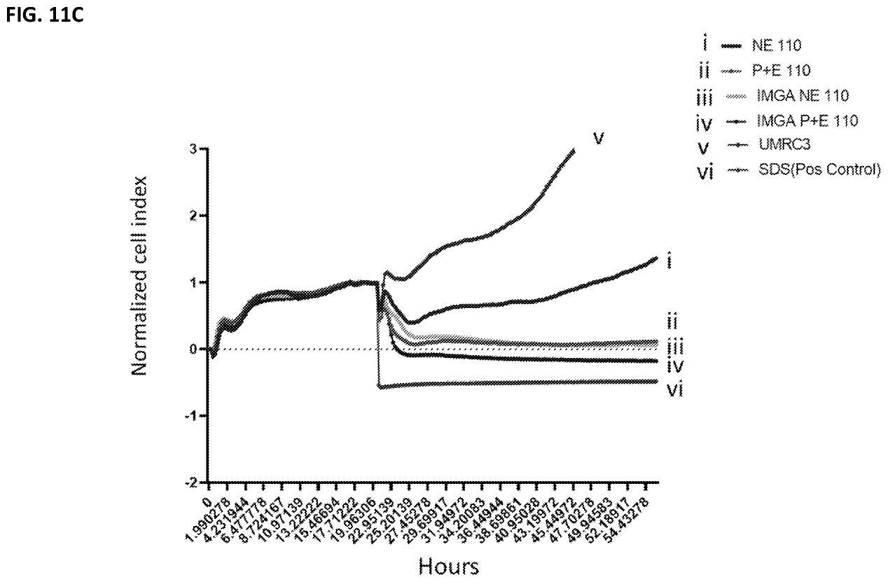

In FIGS. 11A-11C, Imgatuzumab-loaded NK cells show enhanced cytotoxicity against EGFR+ tumor cells. The figure shows an Xcelligence cytotoxicity assay demonstrating normalized cell index of 3 EGFR+ cancer cells lines SKOV3 (ovarian cancer; FIG. 11A), PATC-148 (pancreatic ductal adenocarcinoma; FIG. 11B), and UMRC3 (renal cell carcinoma; FIG. 11C) using 3 different cord blood NK cells (N=3 per assay) at 2:1 effector to target (E:T) ratio. NK cells were either normally expanded (NE) or preactivated and expanded (P+E). NE NK cells were cultured with irradiated (100 Gy) uAPC feeder cells (2:1 feeder cell:NK ratio) and recombinant human IL-2 (200 U/ml) in 50:50 Click's/RPMI media and PE cells were preactivated with IL-12 (10 ng/ml), IL-15 (50 ng/ml), and IL-18 (50 ng/ml) for 16 hrs and then washed and expanded with irradiated uAPC and IL-2 (200 U/ml). NK cells were either left unloaded or loaded with Imgatuzumab 1 μg/ml for one hour at 37° C. in Click's/RPMI media and then washed before the assay. Data indicate that Imgatuzumab loading enhances the cytotoxicity of NE and PE NK cells against tumor cells.

Imgatuzumab-loaded NK cells led to enhanced tumor control in an NSG mouse model of EGFR+ovarian cancer (SKOV3) (FIG. 12). In FIG. 12A, there is one example of a schema of experimental plan for SKOV3 mouse model. FIG. 12B provides bioluminescent imaging showing tumor growth over time in mice engrafted with SKOV3 transduced with Fireflyluciferase (FFluc) and either left untreated or treated with NK cells alone, Imgatuzumab alone or NK cells pre-loaded with Imgatuzumab. In FIG. 12C there is a graph showing average radiance over time of the four groups of mice described in FIG. 12A.

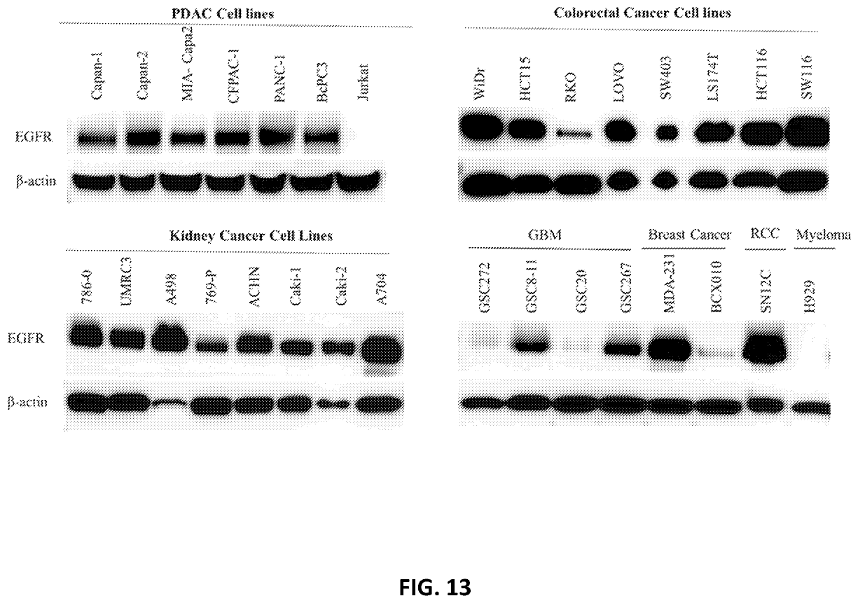

To gauge the potential applicability of treatments described herein that comprise anti-EGFR antibodies (e.g., Imgatuzumab, Amivantamab, etc.), the EGFR expression status was determined for a number of cancer cell lines. The results of these experiments are shown in FIG. 13, where western blot analyses were utilized to determine expression of EGFR (approximately 180 kDa) for various pancreatic adenocarcinoma (PDAC) cell lines, colorectal cancer cell lines, kidney cancer cell lines, GBM cell lines, breast cancer cell lines, renal cancer cell lines, and a myeloma cell line (β-actin loading control). As shown in FIG. 13, EGFR is widely expressed across numerous cancer types, and embodiments of technologies disclosed herein that target EGFR antigens may be widely applicable across cancer types.

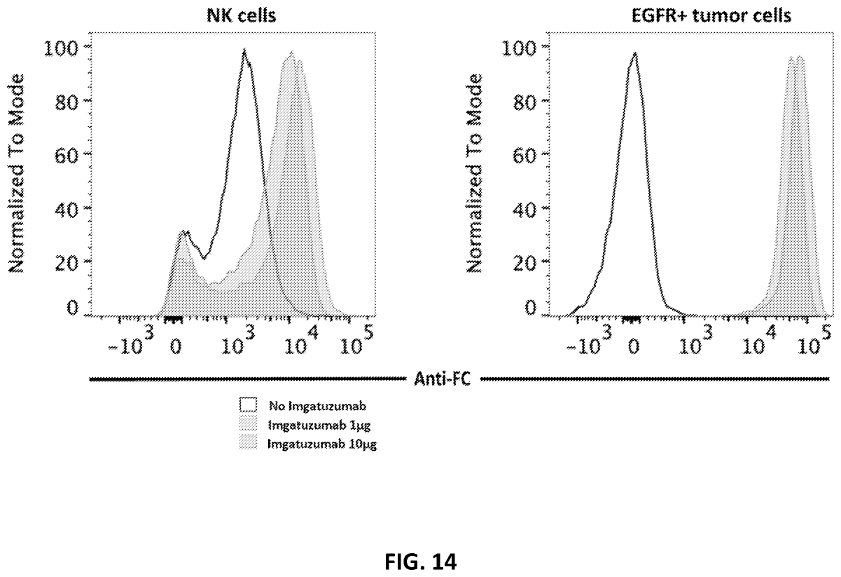

As displayed in FIG. 14, the ability for Imgatuzumab to bind to NK cells and/or EGFR positive tumor cells was determined. In brief, NK cells were derived from cord blood and were expanded with irradiated (100 Gy) UAPC feeder cells (2:1 feeder cell:NK ratio) and recombinant human IL-2 (200 U/ml) in complete serum-free stem cell growth medium (SCGM). As shown in FIG. 14, NK cells (left panel) and tumor cells (right panel) were loaded with Imgatuzumab 1 μg or 10 μg for one hour at 37° C. in SCGM media, and were washed before validating the binding of Imgatuzumab with flow cytometry. NK cells and tumor cells were stained with Alexa-Fluor647 affinity-purified F(ab′)₂ fragment goat anti-human IgG (H+L) antibody and were analyzed using flow cytometry which showed that Imgatuzumab was able to bind to the NK cells and EGFR+tumor cells.

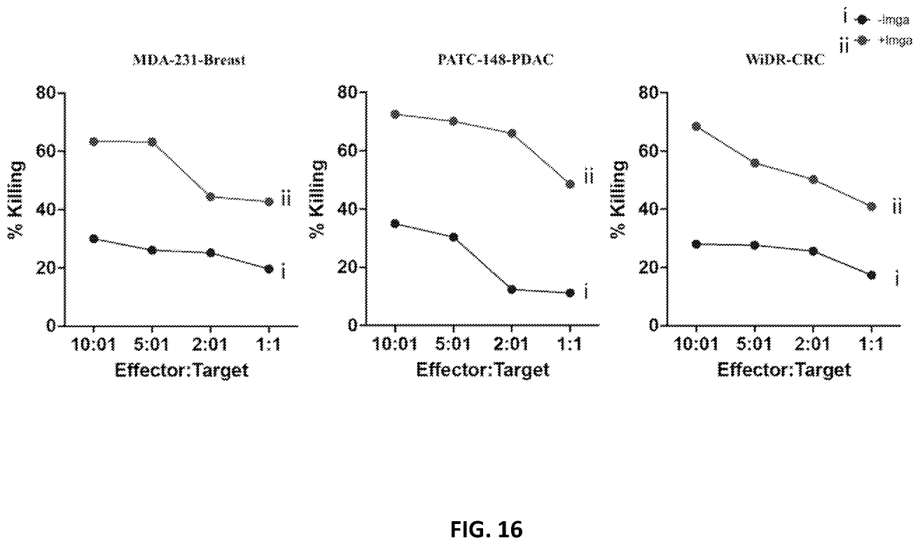

As shown in FIG. 16, Imgatuzumab-loaded NK cells showed enhanced killing of breast, pancreatic (PDAC) and colorectal (CRC) cell lines in vitro when compared to non-loaded NK cells. In brief, NK cells were derived from cord blood were loaded with Imgatuzumab (1 μg) for one hour at 37° C. in SCGM media and washed before co-culturing with tumor cells at various effector to target ratios (10:1, 5:1, 2:1, and 1:1 respectively). Compared to unloaded NK cells, Imgatuzumab loaded NK cells showed increased cytotoxicity against various tumor cells, suggesting that Imgatuzumab loading increased the cytotoxicity activity of NK cells. These results showed that Imgatuzumab loaded NK cells had enhanced cytotoxicity against various cells compared to non-loaded NK cells.

### Example 3-Monoclonal Antibodies and Engineered Nk Cells

The present example encompasses preparation and use of engineered NK cells preloaded with antibodies.

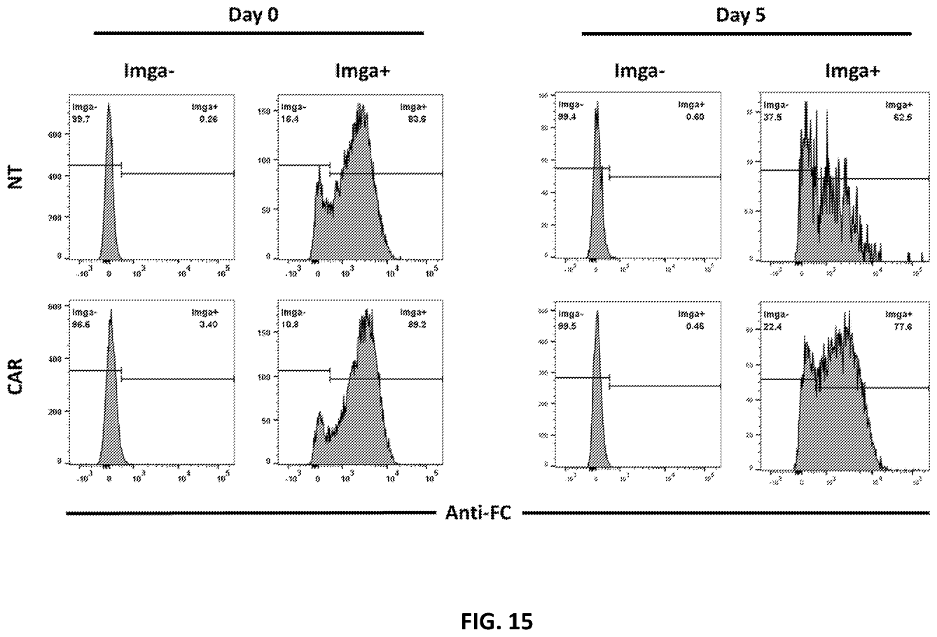

As shown in FIG. 15, Imgatuzumab was found to be retained on NK cells. In brief, NK cells were derived from cord blood and were expanded with irradiated (100 Gy) UAPC feeder cells (2:1 feeder cell:NK ratio) and recombinant human IL-2 (200 U/ml) in complete serum-free SCGM. NK cells were either non-transduced (NT) or transduced with a chimeric antigen receptor (CAR) and were loaded with Imgatuzumab (1 μg) for one hour at 37° C. in SCGM media. Cells were subsequently washed before validating the binding of Imgatuzumab by flow cytometry. NK cells and tumor cells were stained with Alexa-Fluor647 affinity-purified F(ab′)₂ fragment goat anti-human IgG (H+L) antibody at day 0 and day 5 post loading, and were analyzed by flow cytometry. The results showed that Imgatuzumab was bound to the NK cells and was retained on the NK cell surfaces (both engineered CAR transduced NK cells and NT NK cells) for at least 5 days.

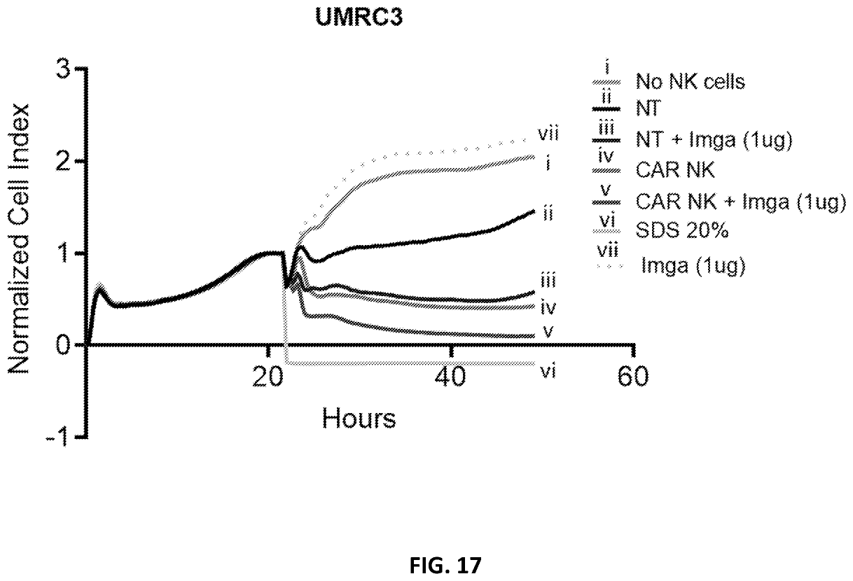

As shown in FIG. 17, Imgatuzumab loaded engineered NK cells (CAR NK+Imga (1 μg)) showed enhanced cytotoxicity against UMRC3 (RCC) tumor cells when compared to non-transduced NK cells (NT), loaded non-engineered NK cells (NT+Imga 1 μg), or non-loaded engineered NK cells (CAR NK). NK cells that were either non-transduced (NT) or transduced (e.g., engineered) with a chimeric antigen receptor (CAR) (e.g., comprising a CD27 CAR targeting CD70, e.g., comprising a CAR according to SEQ ID NO: 2) were loaded with Imgatuzumab (1 μg) for one hour at 37° C. in Click media and then washed before co-culturing with tumor cells at an E:T ratio of 1:1. Compared to non-transduced NK cells (NT), loaded non-engineered NK cells (NT+Imga 1 μg), or non-loaded engineered NK cells (CAR NK), Imgatuzumab loaded engineered NK cells (CAR NK+Imga (1 μg)) showed increased cytotoxicity/tumor cell killing. These results suggested that Imgatuzumab loading coupled with NK cell engineering resulted in a synergistic increase in the cytotoxicity activity of engineered and loaded NK cells relative to either Imgatuzumab treatment alone, non-engineered NK cells, engineered NK cells, or loaded NK cells respectively.

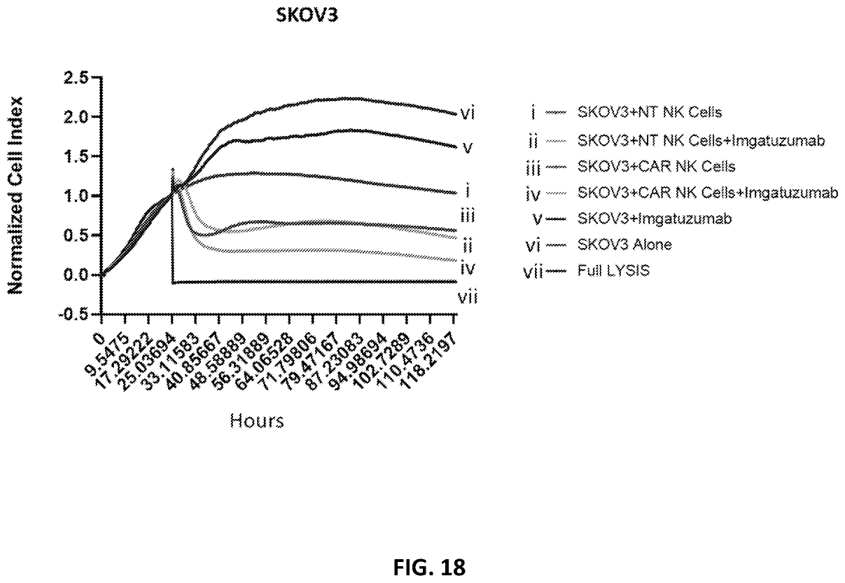

As shown in FIG. 18, Imgatuzumab loaded engineered NK cells (CAR NK+Imga (1 μg)) showed enhanced cytotoxicity against SKOV3 (ovarian Cancer) tumor cells when compared to non-transduced NK cells (NT), loaded non-engineered NK cells (NT+Imga 1 μg), or non-loaded engineered NK cells (CAR NK). NK cells that were either non-transduced (NT) or transduced (e.g., engineered) with a chimeric antigen receptor (CAR) (e.g., comprising a CD27 CAR targeting CD70, e.g., comprising a CAR according to SEQ ID NO: 2) were loaded with Imgatuzumab (1 μg) for one hour at 37° C. in Click's media and then washed before co-culturing with tumor cells at an E:T ratio of 1:1. Compared to non-transduced NK cells (NT), loaded non-engineered NK cells (NT+Imga 1 μg), or non-loaded engineered NK cells (CAR NK), Imgatuzumab loaded engineered NK cells (CAR NK+Imga (1 μg)) showed increased cytotoxicity/tumor cell killing. These results suggested that Imgatuzumab loading coupled with NK cell engineering resulted in a synergistic increase in the cytotoxicity activity of engineered and loaded NK cells relative to either Imgatuzumab treatment alone, non-engineered NK cells, engineered NK cells, or loaded NK cells respectively.

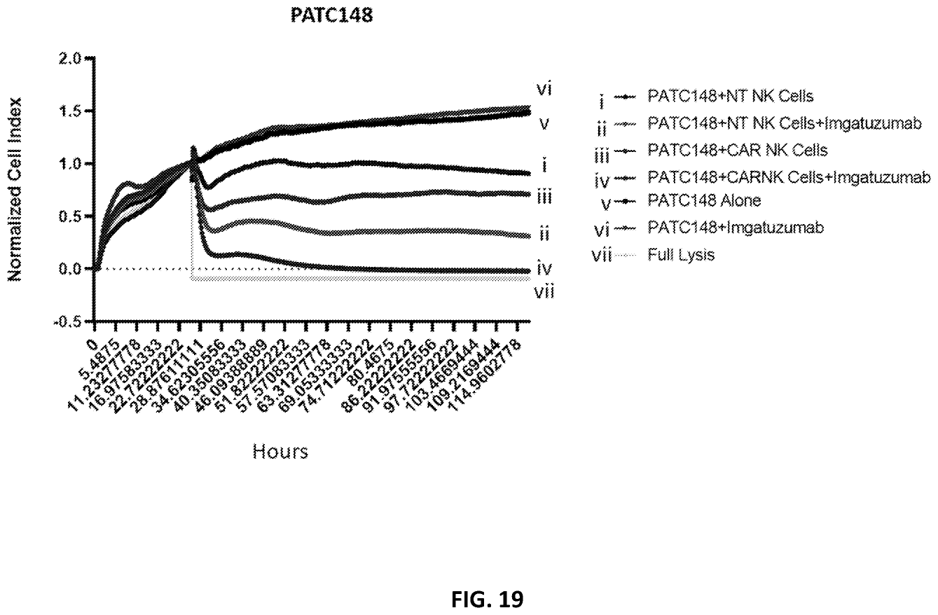

As shown in FIG. 19, Imgatuzumab loaded engineered NK cells (CAR NK+Imga (1 μg)) showed enhanced cytotoxicity against PATC-148 (PDAC) tumor cells when compared to non-transduced NK cells (NT), loaded non-engineered NK cells (NT+Imga 1 μg), or non-loaded engineered NK cells (CAR NK). NK cells that were either non-transduced (NT) or transduced (e.g., engineered) with a chimeric antigen receptor (CAR) (e.g., comprising a CD27 CAR targeting CD70, e.g., comprising a CAR according to SEQ ID NO: 2) were loaded with Imgatuzumab (1 μg) for one hour at 37° C. in Click's media and then washed before co-culturing with tumor cells at an E:T ratio of 1:1. Compared to non-transduced NK cells (NT), loaded non-engineered NK cells (NT+Imga 1 μg), or non-loaded engineered NK cells (CAR NK), Imgatuzumab loaded engineered NK cells (CAR NK+Imga (1 μg)) showed increased cytotoxicity/tumor cell killing. These results suggested that Imgatuzumab loading coupled with NK cell engineering resulted in a synergistic increase in the cytotoxicity activity of engineered and loaded NK cells relative to either Imgatuzumab treatment alone, non-engineered NK cells, engineered NK cells, or loaded NK cells respectively.

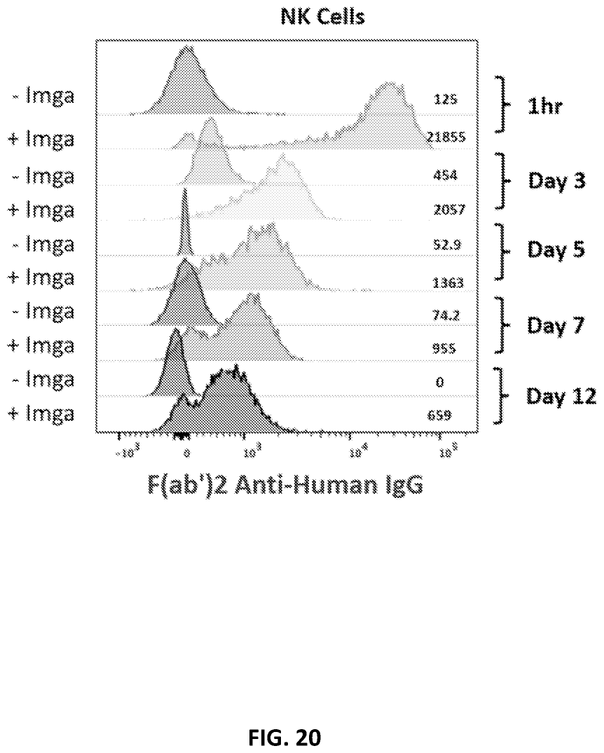

As shown in FIG. 20, antibodies (e.g., Imgatuzumab) were retained on the surface of NK cells for at least 12 days. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI). NK cells were loaded with Imgatuzumab (100 μg/ml final concentration) for one hour at 37° C. in Click's/RPMI media and were washed twice with PBS (5 minutes for each wash) before validating the binding of Imgatuzumab with flow cytometry. NK cells were stained with Alexa-Fluor647 affinity-purified F(ab′)₂ fragment goat anti-human IgG (H+L) antibody at different time points (e.g., 1 hour, 3 days, 4 days, 7 days, and 12 days) and analyzed by flow cytometry. The data showed that Imgatuzumab was retained on surface of the NK cells for at least 12 days following antibody loading.

As shown in FIGS. 21A-21B, Imgatuzumab alone did not cause cytotoxicity against ovarian cancer or PDAC cell lines. Xcelligence assays were performed to assess the cytotoxicity of Imgatuzumab alone against ovarian cell lines (e.g., SKOV3, FIG. 21A) and pancreatic ductal adenocarcinoma (PDAC) cell lines (e.g., PATC148, FIG. 21B). The associated graphs showed normalized cell index (Y axis) plotted over time (X axis, hours). Cell lines cultured alone served as negative controls, and cell lines lysed with SDS served as positive control. The results showed that Imgatuzumab alone failed to impede expansion of SKOV3 or PATC148 cell expansion relative to controls.

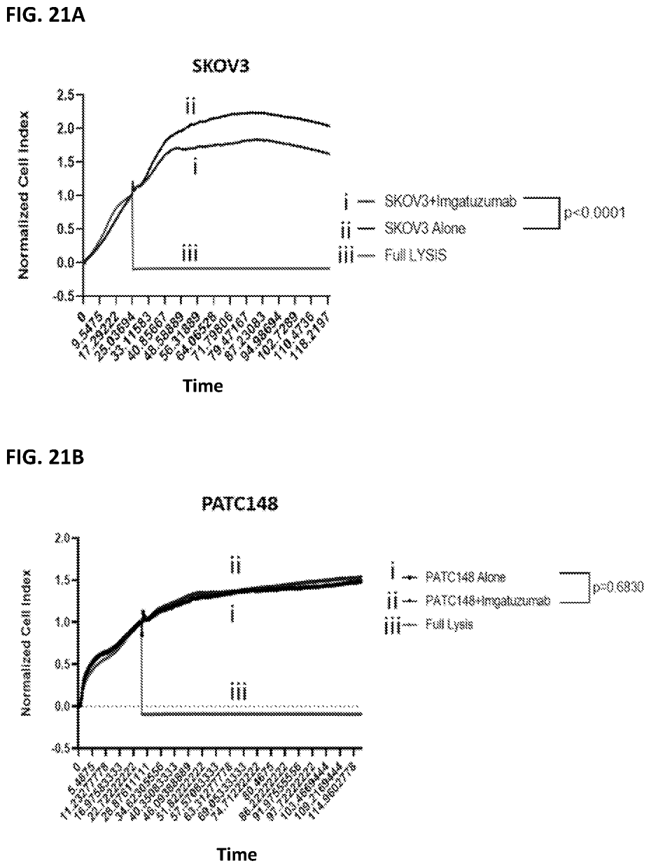

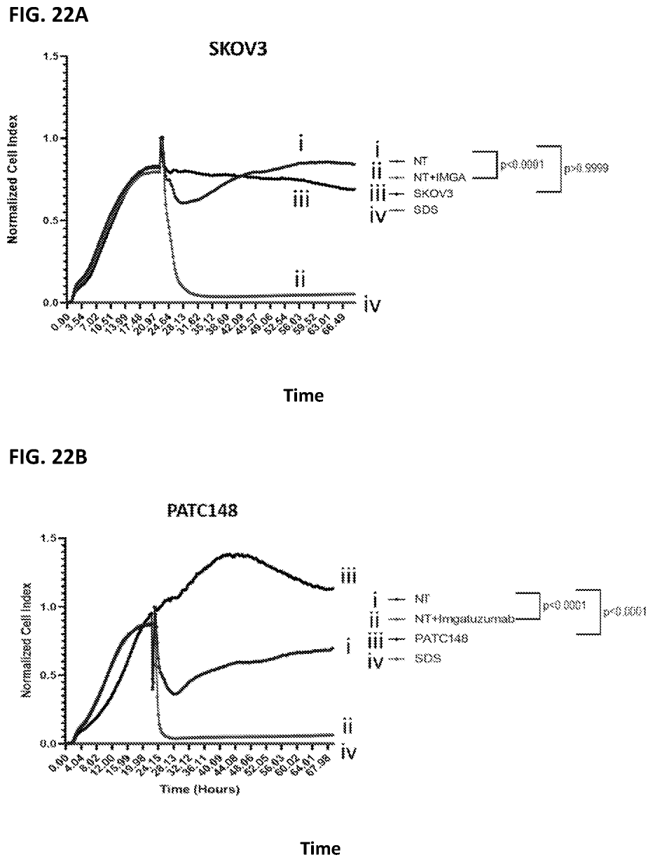

As shown in FIGS. 22A-22B, Imgatuzumab loaded NK cells displayed enhanced killing of ovarian cancer (e.g., SKOV3) cell lines and PDAC (e.g., PATC148) cells when compared to non-loaded NK cells in long-term Xcelligence cytotoxicity assays. NK cells were loaded with Imgatuzumab (100 μg/ml final concentration, e.g., 100 μg of Imgatuzumab in 1 ml of NK cells, e.g., 4 μl of 25 mg/ml stock Imgatuzumab) for one hour at 37° C. in Click's/RPMI media and then washed twice with PBS (5 minutes for each wash) before co-culturing with tumor cells at 1:1 effector to target ratios. When compared to non-loaded NK cells, Imgatuzumab loaded NK cells showed increased cytotoxic activity against SKOV3 (FIG. 22A) and PATC148 (FIG. 22B) cells. Cell lines cultured alone served as negative control and cell line lysed with SDS served as positive control.

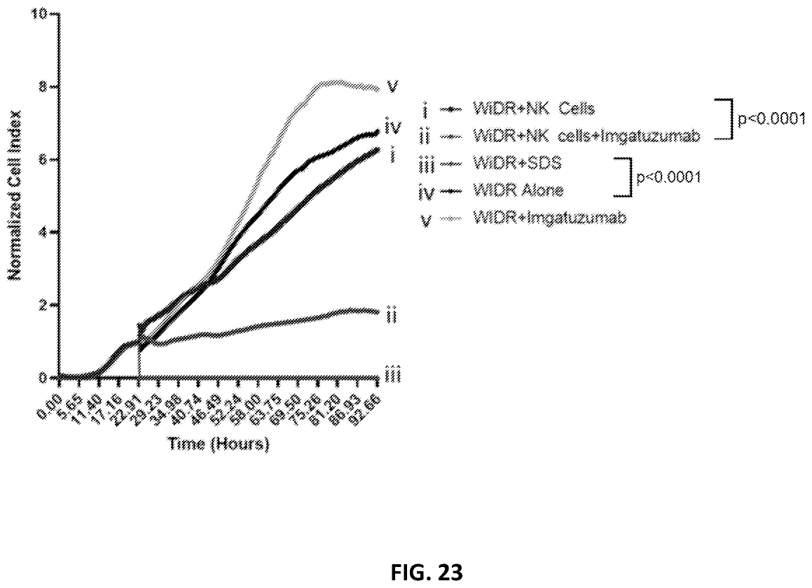

As shown in FIG. 23, Imgatuzumab loaded NK cells displayed enhanced killing of colorectal cancer (CRC) cells (e.g., WiDR) when compared to non-loaded NK cells in long-term Xcelligence cytotoxicity assays. NK cells were loaded with Imgatuzumab (100 μg/ml final concentration) for one hour at 37° C. in Click's/RPMI media and then washed twice with PBS (5 minutes for each wash) before co-culturing with tumor cells at 1:1 effector to target ratio. Compared to non-loaded NK cells or Imgatuzumab alone, Imgatuzumab loaded NK cells showed increased cytotoxic activity, showing that Imgatuzumab loading enhanced the cytotoxic activity of NK cells.

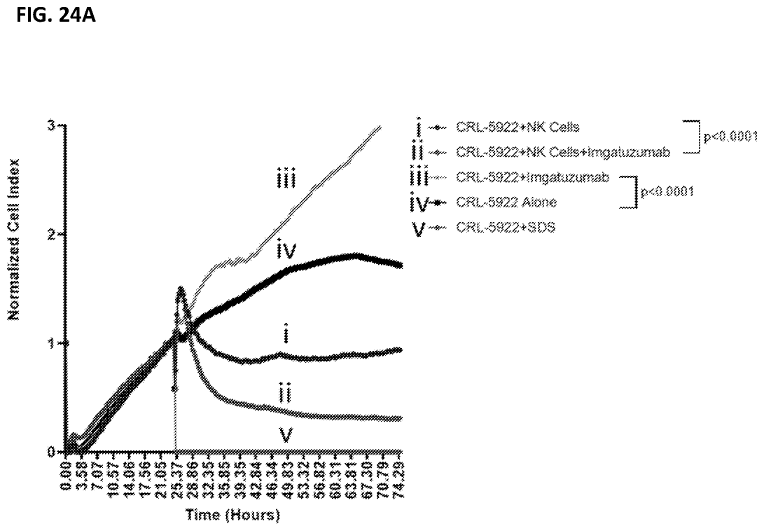

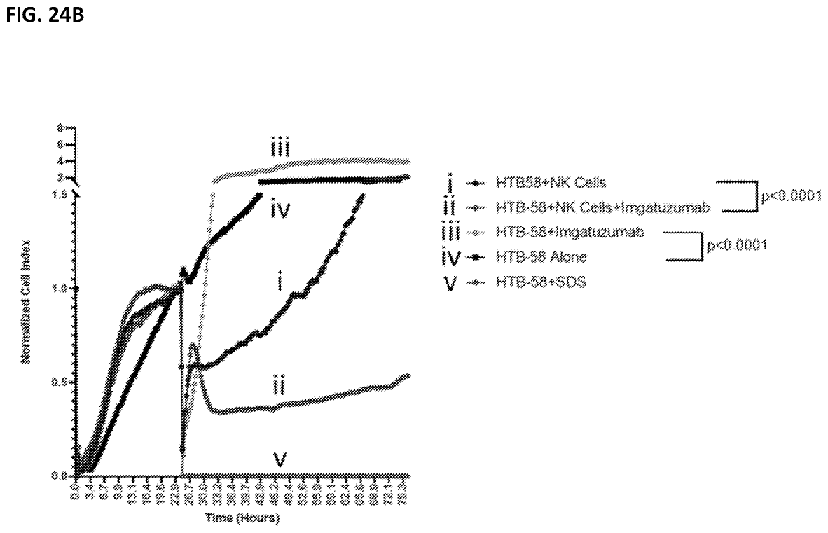

As shown in FIGS. 24A-24B, Imgatuzumab loaded NK cells displayed enhanced killing of lung cancer cells (e.g., CRL-5922 and HTB-58) when compared to non-loaded NK cells in Xcelligence long-term cytotoxicity assays. NK cells were loaded with Imgatuzumab (100 μg/ml final concentration) for one hour at 37° C. in Click's/RPMI media and then washed twice with PBS (5 minutes for each wash) before co-culturing with tumor cells at 1:1 effector to target ratios. Compared to non-loaded NK cells, Imgatuzumab loaded NK cells showed increased cytotoxic activity against CRL-5922 (FIG. 24A) and HTB-58 (FIG. 24B) lung cancer cell lines. Cell lines cultured alone served as negative control and cell line lysed with SDS served as positive control.

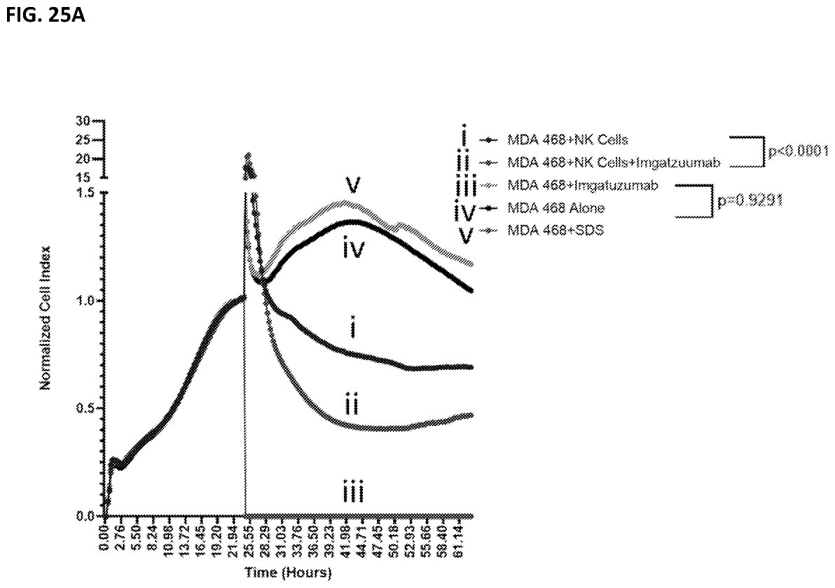

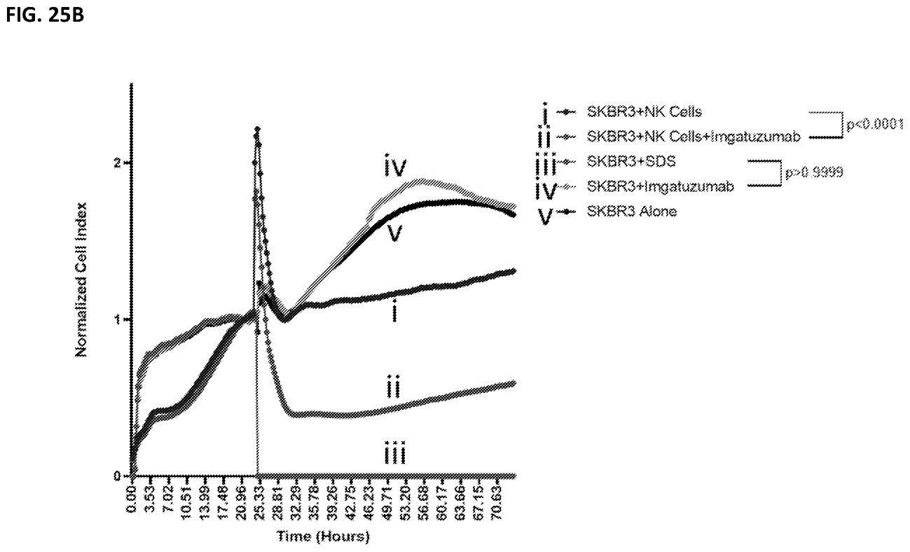

As shown in FIGS. 25A-25B, Imgatuzumab loaded NK cells displayed enhanced killing of breast cancer cells (e.g., MDA 468 and SKBR3) when compared to non-loaded NK cells in Xcelligence long-term cytotoxicity assays. NK cells were loaded with Imgatuzumab (100 μg/ml final concentration) for one hour at 37° C. in Click's/RPMI media and then washed twice with PBS (5 minutes for each wash) before co-culturing with tumor cells at 1:1 effector to target ratios. Compared to non-loaded NK cells, Imgatuzumab loaded NK cells showed increased cytotoxic activity against CRL-5922 (FIG. 25A) and HTB-58 (FIG. 25B) lung cancer cell lines. Cell lines cultured alone served as negative control and cell line lysed with SDS served as positive control.

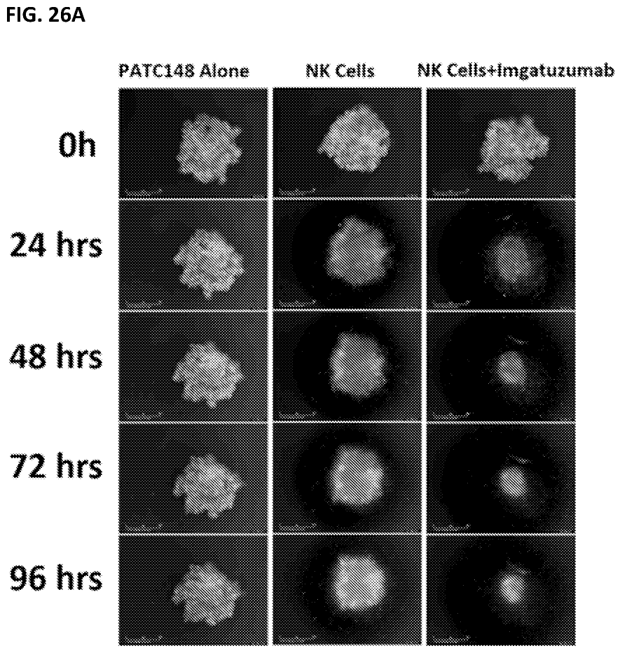

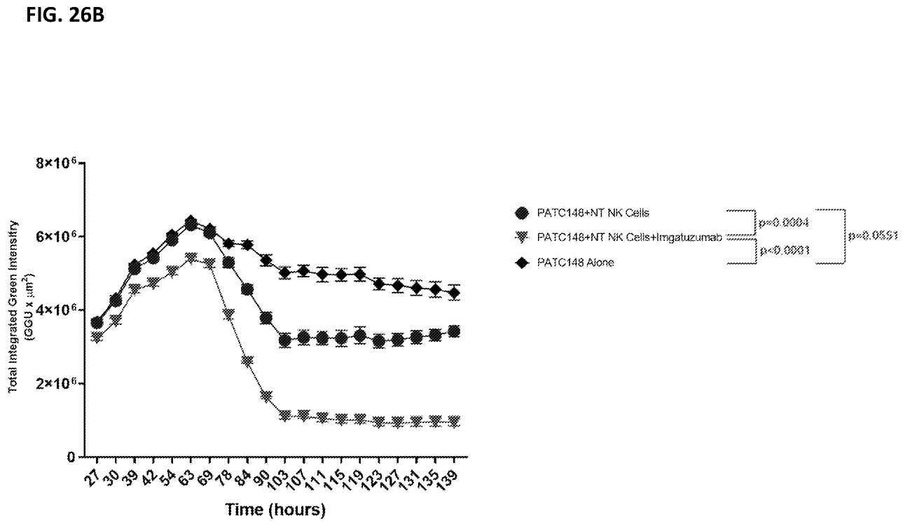

As shown in FIGS. 26A-26B, Imgatuzumab loaded NK cells showed enhanced killing of PDAC (e.g., PATC148) tumor spheroids when compared to non-loaded NK cells in Incucyte live cell imaging assays. FIG. 26A shows representative images of PATC148 spheroids (20,000 tumor cells transduced with GFP and allowed to form spheroids for 48 hours) at different time points, where the tumor cells were left alone or treated with either 50,000 non-loaded NK cells or 50,000 NK cells preloaded with Imgatuzumab (100 μg/ml final concentration). The data showed significant decreases in spheroid size in wells treated with Imgatuzumab loaded NK cells relative to controls. FIG. 26B is a quantification of total integrated green intensity over time, and the data showed significant decreases in total integrated green intensity when spheroids were treated with NK cells loaded with Imgatuzumab when compared to non-loaded NK cells.

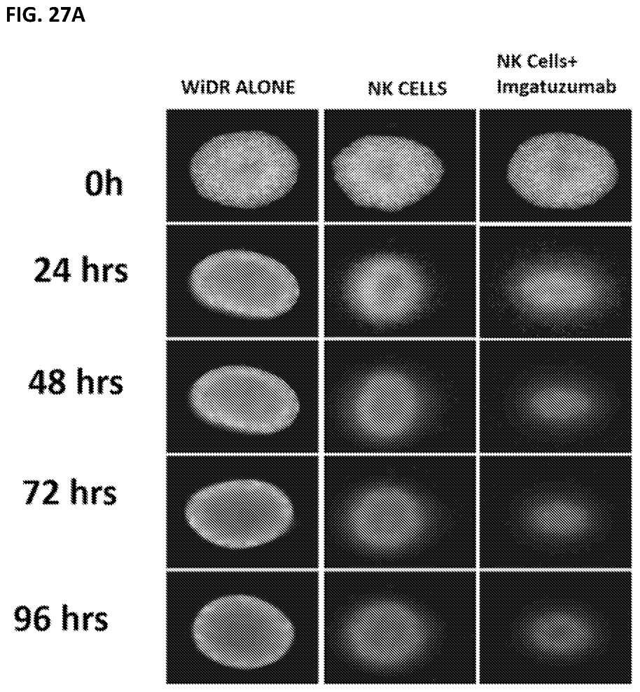

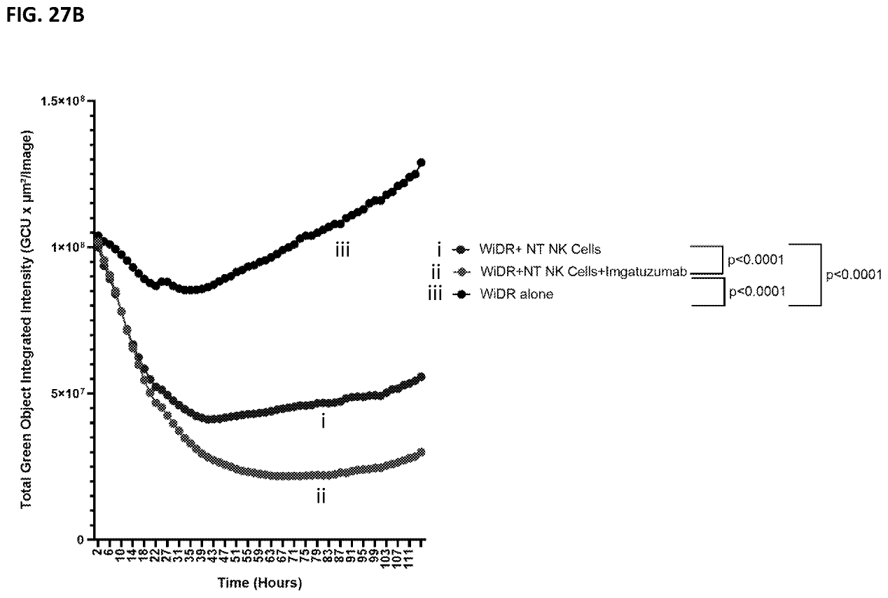

As shown in FIGS. 27A-27B, Imgatuzumab loaded NK cells showed enhanced cytotoxicity against CRC (e.g., WiDR) tumor spheroids when compared to non-loaded NK cells in an Incucyte live cell imaging assay. FIG. 27A shows representative images of WiDR spheroids (20,000 tumor cells transduced with GFP and allowed to form spheroids for 48 hours) left alone or treated with either 50,000 non-loaded NK cells or 50,000 NK cells preloaded with Imgatuzumab (100 μg/ml final concentration) at different time points. The data showed significant decreases in spheroid size in wells treated with Imgatuzumab loaded NK cells. FIG. 27B quantification of total integrated green intensity over time showed significant decreases in total integrated green intensity when spheroids were treated with NK cells loaded with Imgatuzumab when compared to non-loaded NK cells.

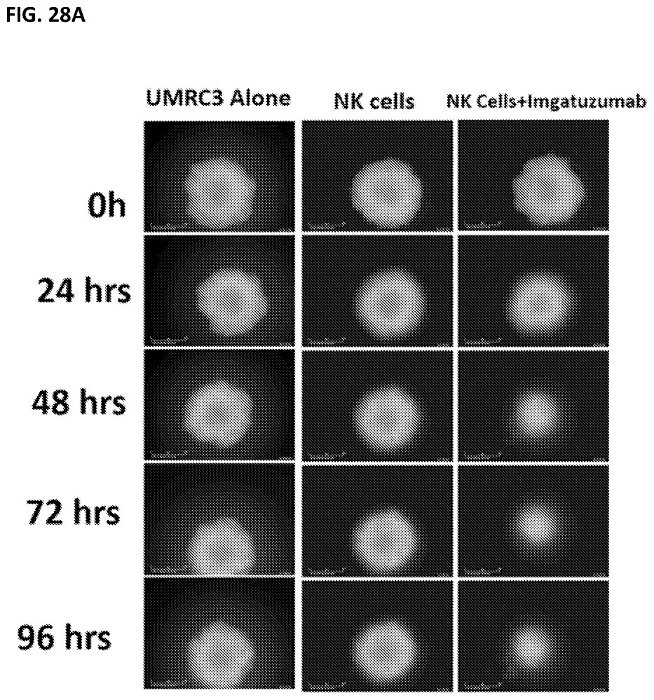

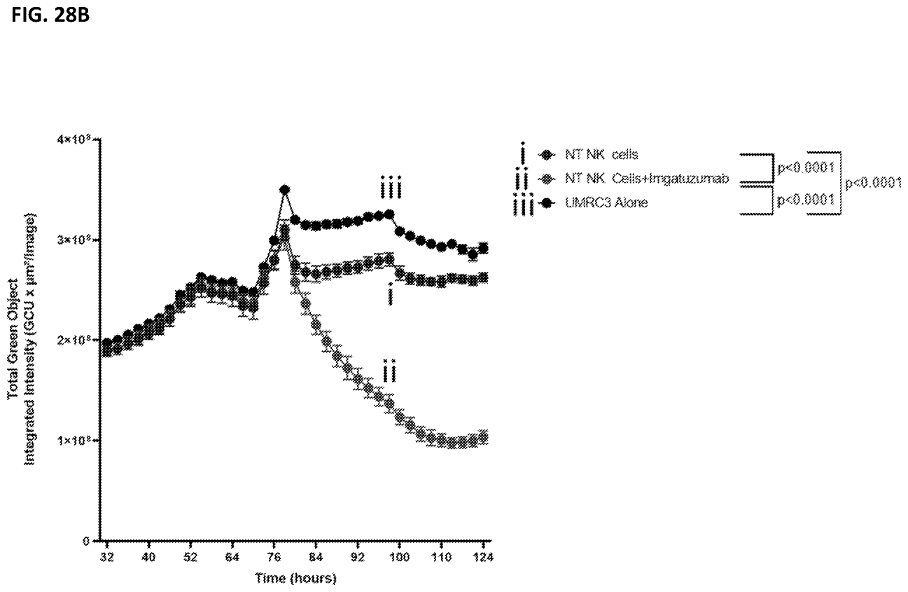

As shown in FIGS. 28A-28B, Imgatuzumab loaded NK cells displayed enhanced cytotoxicity against UMRC3 (RCC) tumor spheroids compared to non-loaded NK cells in an Incucyte live cell imaging assay. FIG. 28A shows a representative images of UMRC3 spheroids (20,000 tumor cells transduced with GFP and allowed to form spheroids for 48 hours) left alone or treated with either 50,000 non-loaded NK cells or 50,000 NK cells preloaded with Imgatuzumab (100 μg/ml final concentration) at different time points. The data showed significant decreases in spheroid size in wells treated with Imgatuzumab loaded NK cells. FIG. 28B quantification of total integrated green intensity over time showed significant decreases in total integrated green intensity when spheroids were treated with NK cells loaded with Imgatuzumab when compared to non-loaded NK cells.

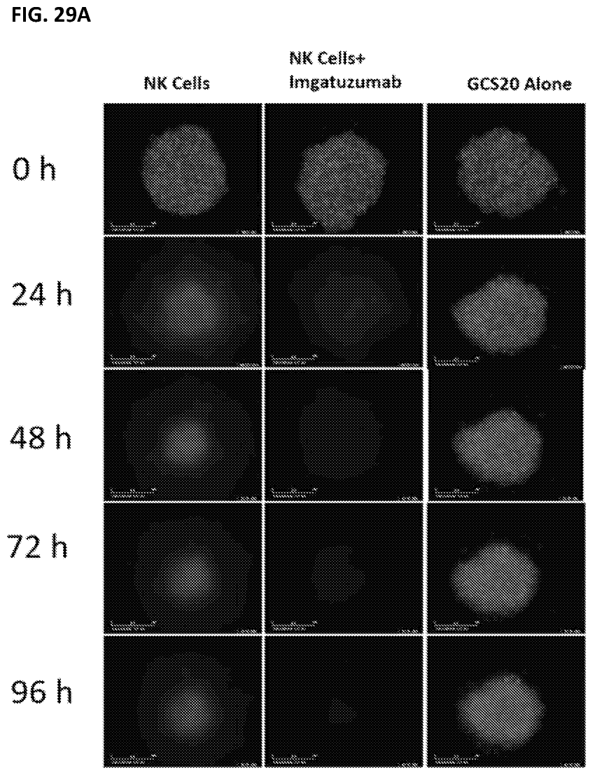

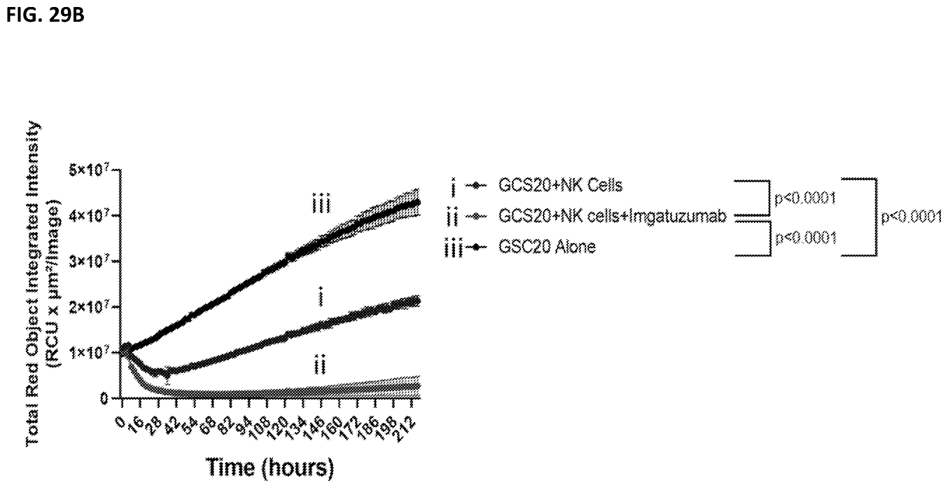

As shown in FIGS. 29A-29B, Imgatuzumab loaded NK cells displayed enhanced cytotoxicity against GBM (e.g., GSC20) tumor spheroids compared to non-loaded NK cells in an Incucyte live cell imaging assay. FIG. 29A shows representative images of GSC20 spheroids (20,000 tumor cells transduced with mCherry and allowed to form spheroids for 48 hours) left alone or treated with either 50,000 non-loaded NK cells or 50,000 NK cells preloaded with Imgatuzumab (100 μg/ml final concentration) at different time points. The data showed significant decreases in spheroid size in wells treated with Imgatuzumab loaded NK cells when compared to non-loaded NK cells. FIG. 29B is quantification of total integrated red intensity over time, and showed significant decreases in total integrated red intensity when spheroids were treated with NK cells loaded with Imgatuzumab when compared to non-loaded NK cells.

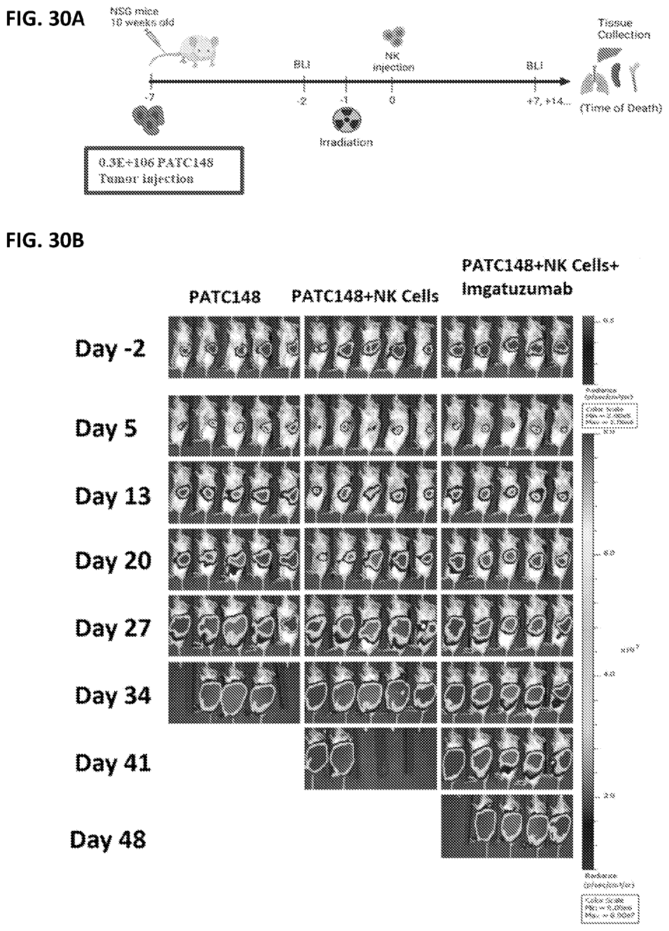

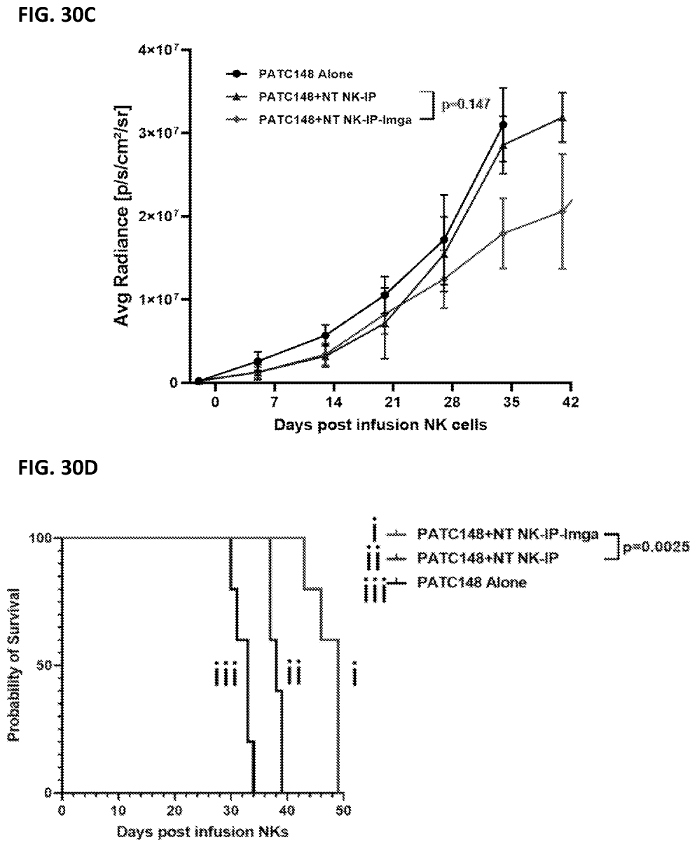

As shown in FIGS. 30A-30D, Imgatuzumab loaded NK cells led to enhanced tumor control in an NSG mouse model of EGFR+PDAC (e.g., PATC148). FIG. 30A is a schema of the experimental plan for a PATC148 mouse model. On day-7 mice 10 week old female NSG mice were injected intravenously (IV) with 0.3×10⁶ PATC148 cells transduced with Fireflyluciferase (FFluc), on day-2 BLI was performed to document engraftment, on day-1 mice were irradiated with 300 cGy, and on day zero, 10⁷ fresh NK cells alone or 10⁷ fresh NK cells pre-loaded with Imgatuzumab (100 μg/ml final concentration), washed twice with saline (5 minutes each wash) and then suspended in saline were injected IV into each mouse. Test mice were then injected with 5 mg/kg Imgatuzumab weekly over three weeks. FIG. 30B shows bioluminescent imaging showing tumor growth over time in mice engrafted with PATC148 transduced with Fireflyluciferase (FFluc) and either left untreated or treated with NK cells alone, or NK cells pre-loaded with Imgatuzumab. FIG. 30C is a graph showing average radiance over time of the 3 groups of mice described in FIG. 30C. FIG. 30D displays survival curves of the 3 groups of mice described in FIG. 30B. Mice treated with NK cells loaded with Imgatuzumab survived significantly longer than the control groups.

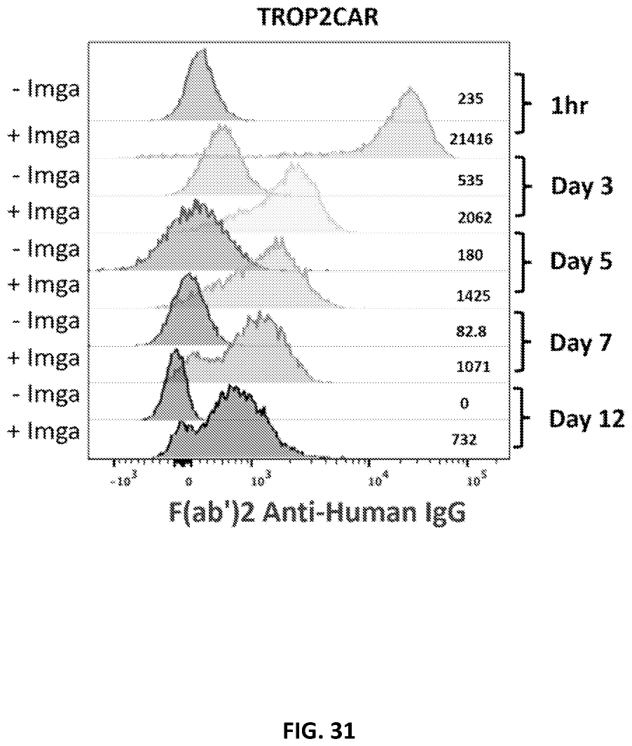

As shown in FIG. 31, Imgatuzumab loaded NT NK cells (as shown in FIG. 20) and TROP2 CAR-NK cells retained antibody on their cell surface for at least 12 days. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI) and either left unmodified non-transduced (NT) or transduced with TROP2 CAR and IL-15 (TROP2) on day 6. NT NK cells or TROP2 CAR NK cells were loaded with Imgatuzumab (100 μg/ml final concentration) for one hour at 37° C. in Click's/RPMI media and were washed twice with PBS (5 minutes for each wash) before validating the binding of Imgatuzumab with flow cytometry. NK cells were stained with Alexa-Fluor647 affinity-purified F(ab′)₂ fragment goat anti-human IgG (H+L) antibody at different time points (1 hour, 3 days, 4 days, 7 days and 12 days) and analyzed by flow cytometry. The results showed that Imgatuzumab was retained on surface of NT NK cells (FIG. 20) and TROP2 CAR NK cells (FIG. 31) for at least 12 days.

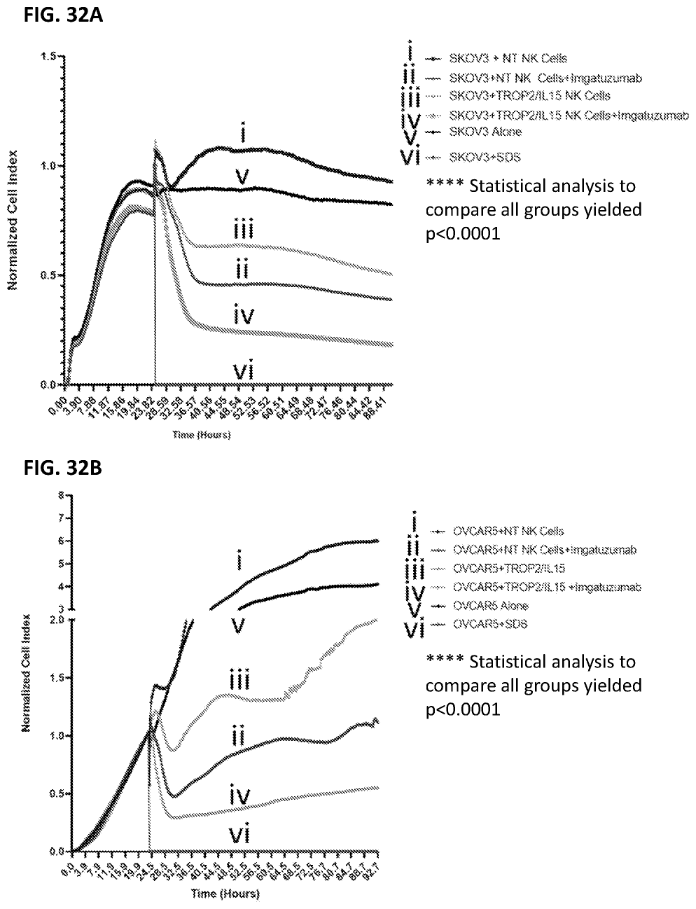

As shown in FIGS. 32A-32B, Imgatuzumab loaded TROP2 CAR-NK cells displayed enhanced killing of ovarian cancer (e.g., SKOV3) cells and gastrointestinal stromal tumor (GIST) (e.g., OVCAR5) cells when compared to non-loaded NK cells in Xcelligence long-term cytotoxicity assays. NK cells either non-transduced (NT) or transduced with TROP2 CAR and IL-15 (TROP2/IL15) were loaded with Imgatuzumab (100 μg/ml final concentration) for one hour at 37° C. in click media and then washed twice with PBS (5 minutes for each wash) before co-culturing with tumor cells at 1:1 effector to target ratio. Compared to non-loaded NK cells, Imgatuzumab loaded NK cells showed increased cytotoxic activity against the target cells. The data showed that Imgatuzumab loading increased the cytotoxic activity of NK cells in both NT NK cells and CAR-NK cells (e.g., TROP2/IL15 CAR NK cells).

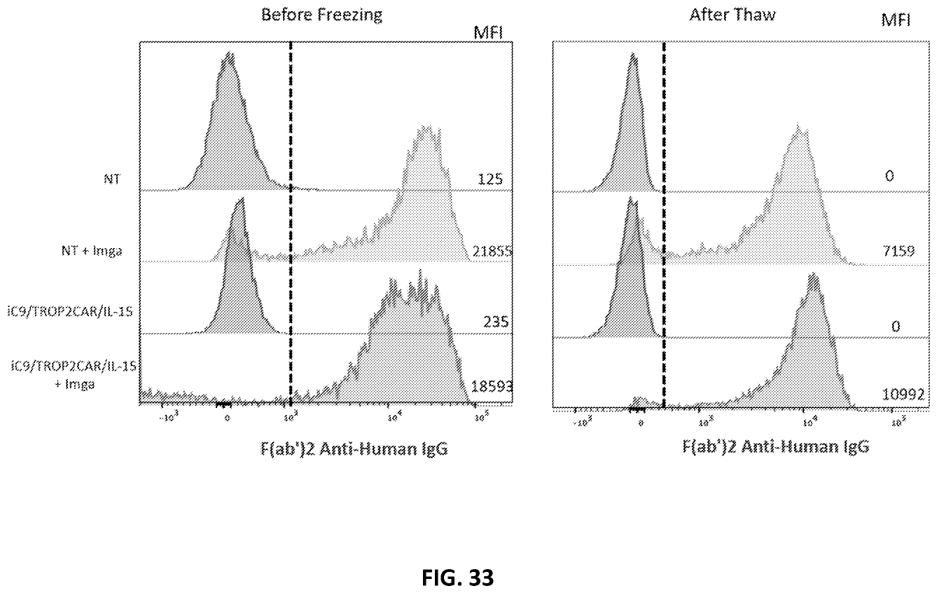

As shown in FIG. 33, Imgatuzumab loaded NK cells maintained antibody on their surface after being frozen and thawed. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI) and either left unmodified non-transduced (NT) or transduced with TROP2 CAR and IL-15 (TROP2CAR/IL-15) on day 6. NT NK cells or TROP2CAR/IL-15 cells were loaded with Imgatuzumab (100 μg/ml final concentration) for one hour at 37° C. in Click's/RPMI media and were washed twice with PBS (5 minutes for each wash) and deactivated with Dasatinib before being frozen. NK cells were then thawed and stained with Alexa-Fluor647 affinity-purified F(ab′)₂ fragment goat anti-human IgG (H+L) antibody and analyzed by flow cytometry. The data showed that Imgatuzumab was retained on surface of the NK cells following freezing and thawing.

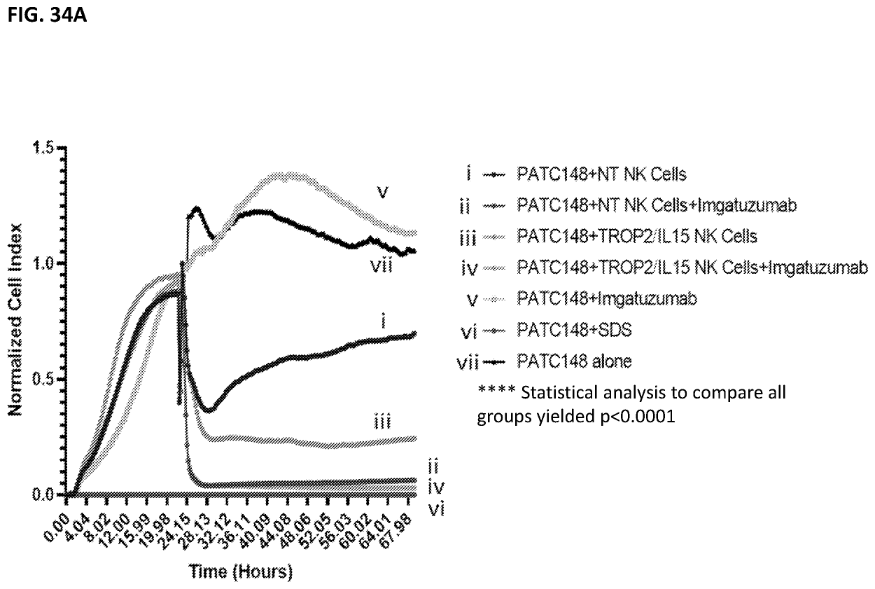

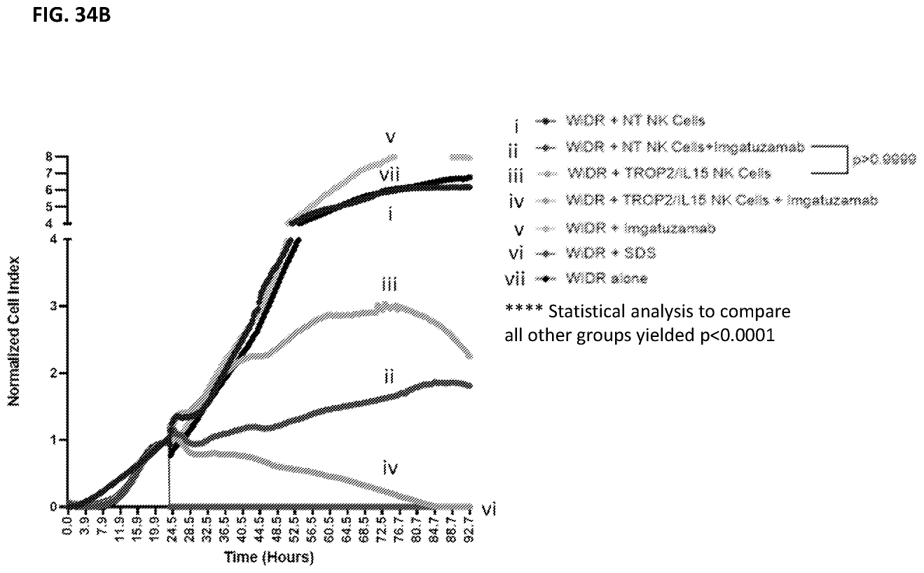

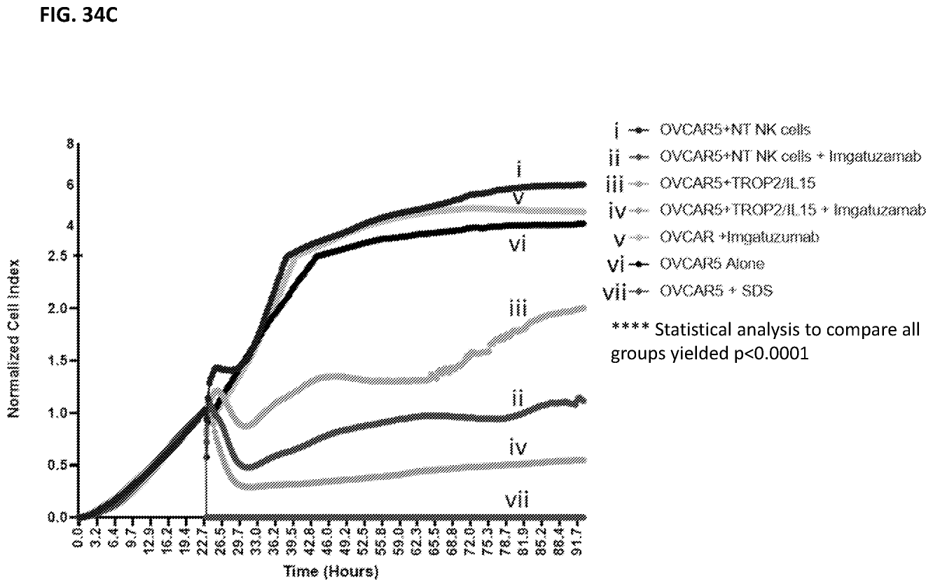

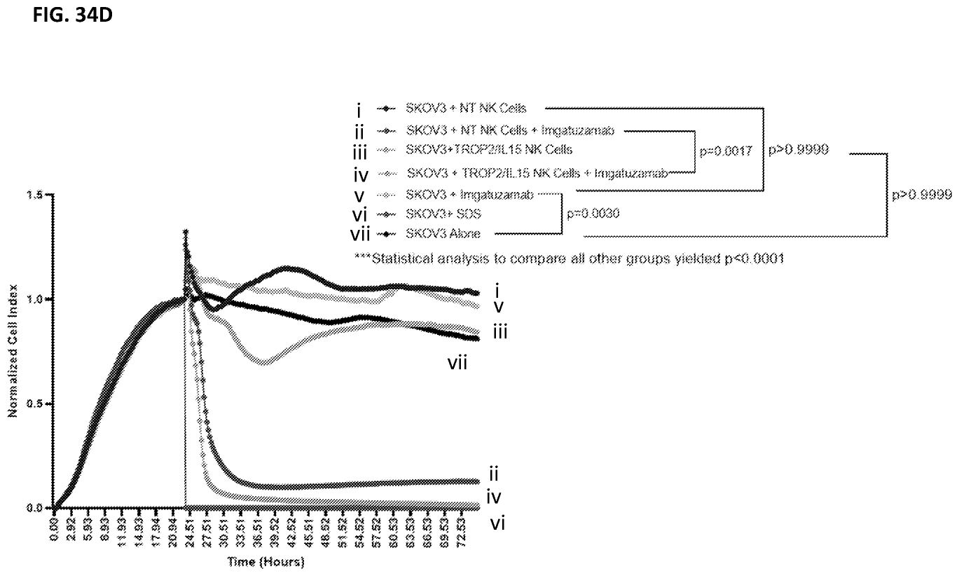

As shown in FIGS. 34A-34D, Imgatuzumab loaded NT and TROP2 CAR-NK cells displayed enhanced cytotoxicity against multiple cancer cell lines when compared to non-loaded NK cells after freeze/thaw. NK cells either non-transduced (NT) or transduced with chimeric antigen receptor TROP2 CAR and IL-15 (TROP2/IL15) were loaded with Imgatuzumab (100 μg/ml final concentration) for one hour at 37° C. in Click's/RPMI media and then washed twice with PBS (5 minutes for each wash), deactivated with Dasatinib, and frozen. Cells were then thawed and used in Xcelligence cytotoxicity assays against multiple cancer cell lines (e.g., PDAC tumor cell line PATC148 (FIG. 34A), CRC cell line WiDR (FIG. 34B), GIST cell line OVCAR5 (FIG. 34C), and ovarian cancer cell line SKOV3 (FIG. 34D)). Compared to non-loaded NK cells, Imgatuzumab loaded NT NK cells and TROP2/IL15 NK cells showed increased cytotoxic activity following freeze/thaw. The data showed that Imgatuzumab loading increased the cytotoxic activity of NK cells in both NT NK cells and TROP2 CAR-NK cells and this enhanced activity was maintained through freeze/thaw.

Engineered CAR NK cells are loaded with Amivantamab (e.g., about 1 to about 10 μg/ml final concentration, or about 10 to 100 μg/ml final concentration, or more), and show enhanced cytotoxicity against tumor cells (e.g., UMRC3, SKOV3, PATC-148, etc.) when engineered NK cells. NK cells are either non-transduced or transduced with a CAR and are loaded with Amivantamab (e.g., about 1 to about 10 μg/ml final concentration, or about 10 to 100 μg/ml final concentration, or more) for about one hour at 37° C. in Click's/RPMI media and are washed (e.g., twice with PBS, 5 minutes for each wash) before co-culturing with tumor cells (e.g., at an E:T ratio of 1:1). Compared to non-transduced NK cells, loaded non-engineered NK cells, and/or non-loaded engineered NK cells, Amivantamab loaded engineered NK cells show an increase in cytotoxicity/tumor cell killing.

Engineered CAR NK cells are loaded with Obinutuzumab (e.g., about 1 to about 10 μg/ml final concentration, or about 10 to 100 μg/ml final concentration, or more), and show enhanced cytotoxicity against tumor cells (e.g., UMRC3, SKOV3, PATC-148, etc.) when compared to non-transduced NK cells, loaded non-engineered NK cells, and/or non-loaded engineered NK cells. NK cells are either non-transduced or transduced with a CAR and are loaded with Obinutuzumab (e.g., about 1 to about 10 μg/ml final concentration, or about 10 to 100 μg/ml final concentration, or more) for about one hour at 37° C. in Click's/RPMI media and are washed (e.g., twice with PBS, 5 minutes for each wash) before co-culturing with tumor cells (e.g., at an E:T ratio of 1:1). Compared to non-transduced NK cells, loaded non-engineered NK cells, and/or non-loaded engineered NK cells, Obinutuzumab loaded engineered NK cells show an increase in cytotoxicity/tumor cell killing.

Engineered CAR NK cells are loaded with Margetuximab (e.g., about 1 to about 10 μg/ml final concentration, or about 10 to 100 μg/ml final concentration, or more), and show enhanced cytotoxicity against tumor cells (e.g., UMRC3, SKOV3, PATC-148, etc.) when compared to non-transduced NK cells, loaded non-engineered NK cells, and/or non-loaded engineered NK cells. NK cells are either non-transduced or transduced with a CAR and are loaded with Margetuximab (e.g., about 1 to about 10 μg/ml final concentration, or about 10 to 100 g/ml final concentration, or more) for about one hour at 37° C. in Click's/RPMI media and are washed (e.g., twice with PBS, 5 minutes for each wash) before co-culturing with tumor cells (e.g., at an E:T ratio of 1:1). Compared to non-transduced NK cells, loaded non-engineered NK cells, and/or non-loaded engineered NK cells, Margetuximab loaded engineered NK cells show an increase in cytotoxicity/tumor cell killing.

Engineered CAR NK cells are loaded with an antibody (e.g., about 1 to about 10 μg/ml final concentration, or about 10 to 100 μg/ml final concentration, or more) and stored for a period of time (e.g., 1, 2, 3, 4, 5, 6, 7, 8, 9, 10, 11, 12, or longer than 12 days), and upon use, show enhanced cytotoxicity against tumor cells (e.g., UMRC3, SKOV3, PATC-148, etc.) when engineered NK cells. NK cells are either non-transduced or transduced with a CAR and are loaded with an antibody (e.g., about 1 to about 10 μg/ml final concentration, or about 10 to 100 μg/ml final concentration, or more) for about one hour at 37° C. in Click's/RPMI media and are washed (e.g., twice with PBS, 5 minutes for each wash) before storage (e.g., 1, 2, 3, 4, 5, 6, 7, 8, 9, 10, 11, 12, or longer than 12 days) and subsequent co-culturing with tumor cells (e.g., at an E:T ratio of 1:1). Compared to stored non-transduced NK cells, loaded non-engineered NK cells, and/or non-loaded engineered NK cells, stored antibody loaded engineered NK cells showed an increase in cytotoxicity/tumor cell killing.

Engineered CAR NK cells are loaded with antibody IPH61 (e.g., about 1 to about 10 μg/ml final concentration, or about 10 to 100 μg/ml final concentration, or more) and stored for a period of time (e.g., 1, 2, 3, 4, 5, 6, 7, 8, 9, 10, 11, 12, or longer than 12 days), and upon use, show enhanced cytotoxicity against CD123 expressing tumor cells when compared to non-transduced NK cells, loaded non-engineered NK cells, and/or non-loaded engineered NK cells. NK cells are either non-transduced or transduced with a CAR and are loaded with an antibody (e.g., about 1 to about 10 μg/ml final concentration, or about 10 to 100 μg/ml final concentration, or more) for about one hour at 37° C. in Click's/RPMI media and are washed (e.g., twice with PBS, 5 minutes for each wash) before storage (e.g., 1, 2, 3, 4, 5, 6, 7, 8, 9, 10, 11, 12, or longer than 12 days) and subsequent co-culturing with CD123 expressing tumor cells (e.g., at an ET ratio of 1:1). Compared to stored non-transduced NK cells, loaded non-engineered NK cells, and/or non-loaded engineered NK cells, stored IPH61 loaded engineered NK cells showed an increase in cytotoxicity/tumor cell killing.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A composition, comprising:
(i) one or more engineered natural killer (NK) cells comprising one or more transgenes; and
(ii) one or more antibody molecules comprising Amivantamab, Margetuximab, and/or Imgatuzumab.

2. The composition of claim 1, wherein the engineered NK cell comes from a population that is or is not expanded.

3. The composition of claim 1 or 2, wherein the engineered NK cell comes from a population that is or is not pre-activated.

4. The composition of any one of claims 1-3, wherein in (i) the composition is further defined as a complex between the engineered NK cell and Margetuximab, Amivantamab, and/or Imgatuzumab, through binding of the Fc region of the antibody to the engineered NK cell.

5. The composition of any one of claims 1-4, wherein the complex further comprises one or more antigen binding domain(s) of the one or more antibody bound to its target antigen.

6. The composition of any one of claims 1-5, wherein the one or more antibody target antigens are Epidermal Growth Factor Receptor (EGFR), Epidermal Growth Factor Receptor-2 (HER2/EGFR-2), and/or tyrosine-protein kinase Met (c-Met).

7. The composition of any one of claims 1-6, wherein the engineered NK cells are derived from NK cells or NK cell precursors obtained from cord blood (CB) mononuclear cells, CB hematopoietic stem cells, peripheral blood (PB) NK cells, bone marrow, stem cells, iPSCs, NK cell lines, or a combination thereof.

8. The composition of any one of claims 1-7, wherein the engineered NK cells are derived from primary NK cells, and are not derived from stem cells and/or induced pluripotent stem cells (iPSCs).

9. The composition of any one of claims 1-8, wherein the NK cells are sourced from cord blood, and wherein the source of the CB mononuclear cells and/or CB hematopoietic stem cells is CB from 1 donor or pooled from 2 or more individual CB units.

10. The composition of any one of claims 1-9, wherein the engineered NK cell is modified to comprise one or more transgenes for expression of one or more heterologous proteins.

11. The composition of claim 10, wherein the one or more heterologous proteins comprises one or more engineered receptors, and/or one or more cytokines.

12. The composition of claim 11, wherein the one or more engineered receptor is a chimeric antigen receptor (CAR), a T cell receptor (TCR), receptors recognizing the FC portion of Ig (FC recognition receptor), a chemokine receptor, a homing receptor, a chimeric cytokine receptor, or any combination thereof.

13. The composition of claim 12, wherein the FC recognition receptor comprises CD16, CD32, and/or CD64, and/or wherein the engineered NK cells express one or more transgenes encoding one or more receptors to enhance their binding to an antibody.

14. The composition of any one of claims 11-13, wherein the engineered receptor comprises a TCR and/or a CAR.

15. The composition of any one of claims 11-14, wherein the engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

16. The composition of any one of claims 11-15, wherein the engineered antigen receptor targets CD70 and/or TROP2.

17. The composition of claim 16, wherein the antigen receptor targeting CD70 comprises a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5.

18. The composition of claim 16 or 17, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4.

19. The composition of any one of claims 16-18, wherein the antigen receptor targeting TROP2 comprises a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9.

20. The composition of any one of claims 16-19, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10.

21. The composition of any one of claims 11-20, wherein the one or more heterologous cytokine comprises IL-2, IL-4, IL-7, IL-12, IL-15, IL-18, IL-21 and/or IL-23.

22. The composition of claim 21, wherein a cytokine comprises IL-15 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12.

23. The composition of claim 21 or 22, wherein a cytokine comprises IL-21 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16.

24. The composition of any one of claims 1-23, wherein the engineered NK cells express one or more transgenes encoding one or more suicide genes.

25. The composition of any one of claims 1-24, wherein the engineered NK cells comprise one or more engineered mutations in an endogenous gene.

26. The composition of claim 25, wherein the engineered mutation comprises a CISH, CD3δ, Glucocorticoid Receptor, and/or TGFBR2 gene mutation.

27. The composition of any one of claims 4-26, wherein complexing of the one or more antibody molecules and NK cells comprises incubation of the NK cells and one or more antibody for at least or exactly about 1 hour prior to washing of the composition to remove unbound antibodies.

28. The composition of any one of claims 4-27, wherein the one or more antibody molecules are incubated with NK cells for formation of a complex at a concentration of about 1 to about 1000 μg/ml final concentration, about 1 μg/ml final concentration, about 10 μg/ml final concentration, about 50 μg/ml final concentration, or about 100 μg/ml final concentration.

29. The composition of claim 27 or 28, wherein the antibody and NK cells are incubated in Click's/RPMI media.

30. The composition of any one of claims 27-29, wherein incubating and washing conditions for complexing of the engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing.

31. The composition of claim 30, wherein relative to the amount of antibodies complexed to the engineered NK cells measured about 1 hour after incubation and washing, at least about 9% of the antibodies are bound to the NK cell surface at least 3 days after incubation and washing.

32. The composition of claim 30, wherein relative to the amount of antibodies complexed to the engineered NK cells measured about 1 hour after incubation and washing, at least about 6% of the antibodies are bound to the NK cell surface at least 5 days after incubation and washing.

33. The composition of claim 30, wherein relative to the amount of antibodies complexed to the engineered NK cells measured about 1 hour after incubation and washing, at least about 4% of the antibodies are bound to the NK cell surface at least 7 days after incubation and washing.

34. The composition of claim 30, wherein relative to the amount of antibodies complexed to the engineered NK cells measured about 1 hour after incubation and washing, at least about 3% of the antibodies are bound to the NK cell surface at least 12 days after incubation and washing.

35. The composition of any one of claims 1-34, wherein the composition has not been frozen, is cryopreserved, or was thawed from cryopreservation.

36. The composition of claim 35, wherein the composition is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

37. The composition of claim 35 or 36, wherein relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

38. The composition of any one of claims 35-36, wherein the engineered NK cells are deactivated prior to cryopreservation.

39. The composition of claim 38, wherein the deactivating agent comprises a kinase inhibitor.

40. The composition of claim 39, wherein the deactivating agent comprises Dasatinib.

41. The composition of any one of claims 1-26, wherein the composition is produced in vivo.

42. The composition of claim 41, wherein production of the composition in vivo comprises loading the engineered NK cells in vivo with antibody by administering the engineered NK cells within about 1 hour of administration of an antibody.

43. The composition of any one of claims 1-42, wherein the one or more antibody comprises or consists of Amivantamab.

44. The composition of any one of claims 1-42, wherein the one or more antibody comprises or consists of Margetuximab.

45. The composition of any one of claims 1-42, wherein the one or more antibody comprises or consists of Imgatuzumab.

46. A method of producing the composition of any one of claims 1-45, comprising the steps of:
(i) optionally expanding engineered NK cells in a culture comprising an effective amount of:
(a) a cytokine selected from the group consisting of IL-2, IL-15, IL-18, IL-21 and a combination thereof; and
(b) antigen presenting cells/feeders, fragments of antigen presenting cells/feeders, or NK cell-activating beads; and
(ii) providing one or more antibody molecules comprising Amivantamab, Margetuximab, and/or Imgatuzumab to the engineered NK cells.

47. The method of claim 46, wherein the method comprises a pre-activating step prior to and/or after the expanding step, wherein the engineered NK cells are pre-activated in a culture comprising an effective concentration of one or more of IL-2, IL-12, IL-15, and IL-18.

48. The method of claim 47, wherein the culture comprises an effective concentration of IL-12, IL-15, and IL-18.

49. The method of any one of claims 46-48, wherein the providing step is further defined as incubating the engineered NK cells with the antibody molecules for a specific duration of time, combining the engineered NK cells and the antibody molecules just prior to infusion, or combining the engineered NK cells and the antibody in vivo in a subject through temporally coordinated administration.

50. The method of claim 49, wherein the duration of time is about 5 minutes to about 24 hours or more.

51. The method of claim 50, wherein the duration of time is about 1 hour.

52. The method of any one of claims 46-51, wherein the culture for the pre-activating step comprises IL-18 and/or IL-15 at a concentration of 0.1-1000 ng/ml, 1-1000 ng/ml, or about 10 ng/mL.

53. The method of any one of claims 46-52, wherein the culture for the pre-activating step comprises IL-12 at a concentration of 0.1-1000 ng/mL, 1-1000 ng/ml, or about 10 ng/mL.

54. The method of any one of claims 46-53, further comprising one or more washing steps of the pre-activated engineered NK cells prior to and/or after the expanding step.

55. The method of any one of claims 46-54, wherein engineered NK cells are activated at least twice or more during the expansion step with IL-12, IL-15, IL-18, IL-2 or any combination thereof.

56. The method of any one of claims 46-55, wherein expanding is for 5-60 days, 12-16 days, or 18-24 days.

57. The method of any one of claims 46-56, wherein the expansion culture further comprises IL-2.

58. The method of claim 57, wherein the IL-2 is present at a concentration of 10-500 U/mL, 100-300 U/mL, or about 200 U/mL.

59. The method of any one of claims 46-58, wherein the IL-2 is replenished in the expansion culture every 2-3 days.

60. The method of any one of claims 46-59, wherein incubating conditions for complexing of the engineered NK cells and one or more antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing.

61. The method of any one of claims 46-60, wherein relative to the amount of antibodies complexed to the engineered NK cells measured about 1 hour after incubation and washing, at least about 9% of the antibodies are bound to the NK cell surface at least 3 days after incubation and washing.

62. The method of any one of claims 46-61, wherein relative to the amount of antibodies complexed to the engineered NK cells measured about 1 hour after incubation and washing, at least about 6% of the antibodies are bound to the NK cell surface at least 5 days after incubation and washing.

63. The method of any one of claims 46-62, wherein relative to the amount of antibodies complexed to the engineered NK cells measured about 1 hour after incubation and washing, at least about 4% of the antibodies are bound to the NK cell surface at least 7 days after incubation and washing.

64. The method of any one of claims 46-63, wherein relative to the amount of antibodies complexed to the engineered NK cells measured about 1 hour after incubation and washing, at least about 3% of the antibodies are bound to the NK cell surface at least 12 days after incubation and washing.

65. The method of any one of claims 46-64, wherein the NK cells provided with one or more antibodies are not frozen, are cryopreserved, or are thawed from cryopreservation.

66. The method of claim 65, wherein the NK cells provided with one or more antibodies are thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

67. The method of claim 65 or 66, wherein relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

68. A method of treating a disease or disorder in a subject, comprising administering a therapeutically effective amount of the compositions of any one of claims 1-45 to the subject.

69. The method of claim 68, wherein the disease or disorder is cancer, inflammation, graft versus host disease, transplant rejection, an autoimmune disorder, an immunodeficiency disease, a B cell malignancy, or an infection.

70. The method of claim 68 or 69, wherein the cancer comprises a hematological cancer or a solid tumor.

71. The method of claim 70, wherein the cancer comprises pancreatic cancer, colorectal cancer, kidney cancer, brain cancer, breast cancer, renal cancer, and/or myeloma.

72. The method of any one of claims 68-71, wherein the engineered NK cells are allogeneic with respect to the subject.

73. The method of any one of claims 68-71, wherein the engineered NK cells are autologous with respect to the subject.

74. The method of any one of claims 68-73, wherein the subject is a human.

75. The method of any one of claims 68-74, further comprising administering at least a second therapeutic agent to the subject.

76. The method of claim 75, wherein the at least a second therapeutic agent is a therapeutically effective amount of one or more anti-cancer agents, one or more immunomodulatory agents, and/or one or more immunosuppressive agents.

77. The method of claim 76, wherein the anti-cancer agent is chemotherapy, radiotherapy, gene therapy, surgery, hormonal therapy, anti-angiogenic therapy or immunotherapy.

78. The method of claim 76, wherein the immunosuppressive agent is a calcineurin inhibitor, an mTOR inhibitor, an antibody, a chemotherapeutic agent irradiation, a chemokine, an interleukins or an inhibitor of a chemokine or an interleukin.

79. The method of any one of claims 75-78, wherein the composition and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

80. The method of any one of claims 75-79, wherein the second therapeutic agent is an antibody.

81. A method of treating a subject with cancer with an engineered NK cell, the method comprising:
a) optionally stimulating a population of engineered NK cells by pre-activation with a cytokine cocktail;
b) optionally expanding the engineered NK cells; and
ci) loading the engineered NK cells ex vivo with one or more antibody and then administering the loaded engineered NK cells to the subject, or
cii) loading the engineered NK cells in vivo with one or more antibody by administering the engineered NK cells within 1 hour of administration of an antibody;
wherein the engineered NK cell comprises one or more transgenes,
wherein the loading comprises exposure of engineered NK cells to the one or more antibody for an amount of time effective for binding of the antibody to the engineered NK cell surface, and
wherein the one or more antibody comprises or consists of Amivantamab, Margetuximab, and/or Imgatuzumab.

82. The method of claim 81, wherein loading the engineered NK cells comprises exposure of the engineered NK cells to the one or more antibody for greater than or equal to about 1 hour, or less than or equal to about 1 hour.

83. The method of claim 81 or 82, wherein engineered NK cells remain loaded with antibody for at least about or about 1 hour, 4 hours, 8 hours, 12 hours, 1 day, 2 days, 3 days, 4 days, 5 days, 6 days, 7 days, 8 days, 9 days, 10 days, 11 days, or 12 days.

84. The method of any one of claims 81-83, wherein the one or more antibody is bound to the engineered NK cell through interaction with a CD16 surface protein.

85. The method of any one of claims 81-84, wherein the engineered NK cells are derived from cord blood.

86. The method of any one of claims 81-85, wherein the engineered NK cells are activated ex-vivo.

87. The method of any one of claims 81-86, wherein the engineered NK cells are pre-activated with IL-18, IL-15, and IL-12.

88. The method of any one of claims 81-87, wherein the one or more transgenes comprise an engineered receptor.

89. The method of claim 88, wherein the engineered receptor comprises a chimeric antigen receptor (CAR), a T cell receptor, chemokine receptor, chimeric cytokine receptor, or any combination thereof.

90. The method of claim 88 or 89, wherein the engineered receptor comprises a CAR or a TCR.

91. The method of any one of claims 88-90, wherein the engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

92. The method of claim 91, wherein the engineered antigen receptor targets CD70 and/or TROP2.

93. The method of claim 91 or 92, wherein an antigen receptor targeting CD70 comprises a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5.

94. The method of claim 92 or 93, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs:
1, 3, or 4.

95. The method of any one of claims 91-94, wherein an antigen receptor targeting TROP2 comprises a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9.

96. The method of any one of claims 91-95, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10.

97. The method of any one of claims 81-96, wherein the one or more transgenes comprise a cytokine.

98. The method of claim 97 wherein the one or more heterologous cytokine comprises IL-2, IL-4, IL-7, IL-12, IL-15, IL-18, IL-21 and/or IL-23.

99. The method of claim 98, wherein the cytokine comprises IL-2, IL-12, IL-15, IL-18, and/or IL-21.

100. The method of claim 99, wherein a cytokine comprises or consists of IL-15 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12.

101. The method of claim 99 or 100, wherein a cytokine comprises or consists of IL-21 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16.

102. The method of any one of claims 81-101, wherein the engineered NK cells comprise on or more engineered mutations in an endogenous gene.

103. The method of claim 102, wherein the one or more engineered mutation in an endogenous gene comprises a CISH, CD3δ, Glucocorticoid Receptor, and/or TGFBR2 gene mutation.

104. A composition, comprising:
(i) one or more engineered natural killer (NK) cells comprising one or more transgenes; and
(ii) one or more antibodies comprising antibody IPH61.

105. The composition of claim 104 or 105, wherein the antibody targets at least CD123 and NKp46.

106. The composition of any one of claims 104-105, wherein the composition is further defined as a complex between the engineered NK cell IPH61 through binding of the Fc region of the antibody to the engineered NK cell and/or binding of the NKp46 targeting region of the antibody to the engineered NK cell.

107. The composition of any one of claims 104-106, wherein the complex further comprises one or more antigen binding domain(s) of the one or more antibody bound to its target antigen.

108. The composition of any one of claims 104-10⁷, wherein the engineered NK cells are derived from NK cells or NK cell precursors obtained from cord blood (CB) mononuclear cells, CB hematopoietic stem cells, peripheral blood (PB) NK cells, bone marrow, stem cells, iPSCs, NK cell lines, or a combination thereof.

109. The composition of any one of claims 104-108, wherein the engineered NK cell is modified to comprise one or more transgenes for expression of one or more heterologous proteins.

110. The composition of claim 109, wherein the one or more heterologous proteins comprises one or more engineered receptors, and/or one or more cytokines.

111. The composition of claim 110, wherein the one or more engineered receptor is a chimeric antigen receptor (CAR), a T cell receptor (TCR), receptors recognizing the FC portion of Ig (FC recognition receptor), a chemokine receptor, a homing receptor, a chimeric cytokine receptor, or any combination thereof.

112. The composition of claim 111, wherein the FC recognition receptor comprises CD16, CD32, and/or CD64, and/or wherein the engineered NK cells express one or more transgenes encoding one or more receptors and/or NK cell surface antigens to enhance NK cell binding to an antibody.

113. The composition of claim 110, wherein the engineered NK cell surface antigen and/or receptor is CD16, CD32, CS1, CD56, CD64, NKG2D, NKG2C, DNAM, 2B4, CD2, an NCR, NKp30, NKp44, NKp46, or KIR.

114. The composition of any one of claims 110-113, wherein the engineered receptor comprises a TCR and/or a CAR.

115. The composition of any one of claims 110-114, wherein the engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

116. The composition of any one of claims 110-115, wherein the engineered antigen receptor targets CD70 and/or TROP2.

117. The composition of claim 116, wherein the antigen receptor targeting CD70 comprises a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5.

118. The composition of claim 116 or 117, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4.

119. The composition of any one of claims 116-118, wherein the antigen receptor targeting TROP2 comprises a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9.

120. The composition of any one of claims 116-119, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10.

121. The composition of any one of claims 116-120, wherein the one or more heterologous cytokine is IL-2, IL-4, IL-7, IL-12, IL-15, IL-18, IL-21 and/or IL-23.

122. The composition of claim 121, wherein a cytokine comprises IL-15 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12.

123. The composition of claim 121 or 122, wherein a cytokine comprises IL-21 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 13-16.

124. The composition of any one of claims 104-123, wherein the engineered NK cells express one or more transgenes encoding one or more suicide genes.

125. The composition of any one of claims 104-124, wherein the engineered NK cells comprise one or more engineered mutations in an endogenous gene.

126. The composition of claim 125, wherein the one or more engineered mutations comprises a CISH, CD3δ, Glucocorticoid Receptor, and/or TGFBR2 gene mutation.

127. The composition of any one of claims 106-126, wherein complexing of the one or more antibodies comprising IPH61 and NK cells comprises incubation of the NK cells and one or more antibody for at least or exactly about 1 hour prior to washing of the composition to remove unbound antibodies.

128. The composition of any one of claims 106-127, wherein the one or more antibody molecules are incubated with NK cells for formation of a complex at a concentration of about 1 to about 1000 μg/ml final concentration, about 1 μg/ml final concentration, about 10 μg/ml final concentration, about 50 μg/ml final concentration, or about 100 μg/ml final concentration.

129. The composition of claim 127 or 128, wherein the antibody and NK cells are incubated in Click's/RPMI media.

130. The composition of any one of claims 127-129, wherein incubating and washing conditions for complexing of the engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing.

131. The composition of any one of claims 104-130, wherein the composition has not been frozen, is cryopreserved, or was thawed from cryopreservation.

132. The composition of claim 131, wherein the composition is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

133. The composition of claim 131 or 132, wherein relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

134. The composition of any one of claims 131-133, wherein the engineered NK cells are deactivated prior to cryopreservation.

135. The composition of claim 134, wherein the deactivating agent comprises a kinase inhibitor.

136. The composition of claim 135, wherein the deactivating agent comprises Dasatinib.

137. The composition of any one of claims 104-136, wherein the composition is produced in vivo.

138. The composition of claim 137, wherein production of the composition in vivo comprises loading the engineered NK cells in vivo with one or more antibody by administering the engineered NK cells within about 1 hour of administration of an antibody.

139. A method of producing the composition of any one of claims 104-138, comprising the steps of:
(i) optionally expanding engineered NK cells in a culture comprising an effective amount of:
(a) a cytokine selected from the group consisting of IL-2, IL-15, IL-18, IL-21 and a combination thereof; and
(b) antigen presenting cells/feeders, fragments of antigen presenting cells/feeders, or NK cell-activating beads; and
(ii) providing one or more antibody molecules comprising IPH61 to the engineered NK cells.

140. The method of claim 139, wherein the method comprises a pre-activating step prior to and/or after the expanding step, wherein the engineered NK cells are pre-activated in a culture comprising an effective concentration of one or more of IL-2, IL-12, IL-15, and IL-18.

141. The method of claim 140, wherein the culture comprises an effective concentration of IL-12, IL-15, and IL-18.

142. The method of any one of claims 139-141, wherein the providing step is further defined as incubating the engineered NK cells with the antibody molecules for a specific duration of time, combining the engineered NK cells and the antibody molecules just prior to infusion, or combining the engineered NK cells and the antibody in vivo in a subject through temporally coordinated administration.

143. The method of claim 142, wherein the duration of time is about 5 minutes to about 24 hours or more.

144. The method of claim 143, wherein the duration of time is about 1 hour.

145. The method of any one of claims 139-144, wherein the culture for the pre-activating step comprises IL-18 and/or IL-15 at a concentration of 0.1-1000 ng/ml, 1-1000 ng/mL, or about 10 ng/mL.

146. The method of any one of claims 139-145, wherein the culture for the pre-activating step comprises IL-12 at a concentration of 0.1-1000 ng/ml, 1-1000 ng/ml, or about 10 ng/mL.

147. The method of any one of claims 139-146, wherein expanding is for 5-60 days, 12-16 days, or 18-24 days.

148. The method of any one of claims 139-147, wherein the expansion culture further comprises IL-2.

149. The method of claim 148, wherein the IL-2 is present at a concentration of 10-500 U/mL, 100-300 U/mL, or about 200 U/mL.

150. The method of any one of claims 139-149, wherein incubating conditions for complexing of the engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing.

151. The method of any one of claims 139-150, wherein the NK cells provided with antibodies are not frozen, are cryopreserved, or are thawed from cryopreservation.

152. The method of claim 151, wherein the NK cells provided with antibodies are thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

153. The method of claim 151 or 152, wherein relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

154. A method of treating a disease or disorder in a subject, comprising administering a therapeutically effective amount of the compositions of any one of claims 104-138 to the subject.

155. The method of claim 154, wherein the disease or disorder is cancer, inflammation, graft versus host disease, transplant rejection, an autoimmune disorder, an immunodeficiency disease, a B cell malignancy, or an infection.

156. The method of claim 154 or 155, wherein the cancer comprises a hematological cancer or a solid tumor.

157. The method of claim 156, wherein the cancer comprises acute myeloid leukemia, blastic plasmacytoid dendritic cell neoplasm, acute lymphoblastic leukemia, hairy cell leukemia, and/or systemic mastocytosis.

158. The method of any one of claims 154-158, wherein the engineered NK cells are allogeneic with respect to the subject.

159. The method of any one of claims 154-158, wherein the engineered NK cells are autologous with respect to the subject.

160. The method of any one of claims 154-159, wherein the subject is a human.

161. The method of any one of claims 154-160, further comprising administering at least a second therapeutic agent to the subject.

162. The method of claim 161, wherein the at least a second therapeutic agent is a therapeutically effective amount of one or more anti-cancer agents, one or more immunomodulatory agents, and/or one or more immunosuppressive agents.

163. A composition, comprising:
(i) one or more engineered natural killer (NK) cells comprising one or more transgenes; and
(ii) one or more antibody molecules,
wherein the one or more transgenes comprise a CD70 targeting CAR comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5, and/or
wherein the one or more transgenes comprise a TROP2 targeting CAR comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9.

164. The composition of claim 163, wherein the engineered NK cell comes from a population that is or is not expanded.

165. The composition of claim 163 or 164, wherein the engineered NK cell comes from a population that is or is not pre-activated.

166. The composition of any one of claims 163-165, wherein the composition is further defined as a complex between the engineered NK cell and one or more antibodies.

167. The composition of any one of claims 163-166, wherein the complex further comprises one or more antigen binding domain(s) of the one or more antibody bound to its target antigen.

168. The composition of any one of claims 163-167, wherein the antibody targets a cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

169. The composition of any one of claims 163-168, wherein the one or more antibody targets Epidermal Growth Factor Receptor (EGFR), Epidermal Growth Factor Receptor-2 (HER2/EGFR-2), NKp46, CD123, and/or tyrosine-protein kinase Met (c-Met).

170. The composition of any one of claims 163-169, wherein the engineered NK cells are derived from NK cells or NK cell precursors obtained from cord blood (CB) mononuclear cells, CB hematopoietic stem cells, peripheral blood (PB) NK cells, bone marrow, stem cells, iPSCs, NK cell lines, or a combination thereof.

171. The composition of any one of claims 163-170, wherein the engineered NK cells are derived from primary NK cells, and are not derived from stem cells and/or induced pluripotent stem cells (iPSCs).

172. The composition of any one of claims 163-171, wherein the NK cells are sourced from cord blood, and wherein the source of the CB mononuclear cells and/or CB hematopoietic stem cells is CB from 1 donor or pooled from 2 or more individual CB units.

173. The composition of any one of claims 163-172, wherein the engineered NK cell is further modified to comprise one or more additional transgenes for expression of one or more heterologous proteins.

174. The composition of claim 173, wherein the one or more heterologous proteins comprises one or more engineered receptors, and/or one or more cytokines.

175. The composition of claim 174, wherein the one or more engineered receptor is a chimeric antigen receptor (CAR), a T cell receptor (TCR), receptors recognizing the FC portion of Ig (FC recognition receptor), a chemokine receptor, a homing receptor, a chimeric cytokine receptor, or any combination thereof.

176. The composition of claim 175, wherein the FC recognition receptor comprises CD16, CD32, and/or CD64, and/or wherein the engineered NK cells express one or more transgenes encoding one or more receptors to enhance their binding to an antibody.

177. The composition of any one of claims 174-176, wherein the engineered receptor comprises a TCR and/or a CAR.

178. The composition of any one of claims 174-177, wherein the engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

179. The composition of any one of claims 163-178, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4.

180. The composition of any one of claims 163-179, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6, 8, or 10.

181. The composition of any one of claims 174-180, wherein the one or more heterologous cytokine comprises IL-2, IL-4, IL-7, IL-12, IL-15, IL-18, IL-21 and/or IL-23.

182. The composition of claim 181, wherein a cytokine comprises IL-15 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 11 or 12.

183. The composition of claim 181 or 182, wherein a cytokine comprises IL-21 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16.

184. The composition of any one of claims 163-183, wherein the engineered NK cells express one or more transgenes encoding one or more suicide genes.

185. The composition of any one of claims 163-184, wherein the engineered NK cells comprise one or more engineered mutations in an endogenous gene.

186. The composition of claim 185, wherein the engineered mutation comprises a CISH, CD3δ, Glucocorticoid Receptor, and/or TGFBR2 gene mutation.

187. The composition of any one of claims 166-186, wherein complexing of the one or more antibody molecules and NK cells comprises incubation of the NK cells and one or more antibody for at least or exactly about 1 hour prior to washing of the composition to remove unbound antibodies.

188. The composition of any one of claims 166-187, wherein the one or more antibody molecules are incubated with NK cells for formation of a complex at a concentration of about 1 to about 1000 μg/ml final concentration, about 1 μg/ml final concentration, about 10 μg/ml final concentration, about 50 μg/ml final concentration, or about 100 μg/ml final concentration.

189. The composition of claim 187 or 188, wherein the antibody and NK cells are incubated in Click's/RPMI media.

190. The composition of any one of claims 187-189, wherein incubating and washing conditions for complexing of the engineered NK cells and antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing.

191. The composition of claim 190, wherein relative to the amount of antibodies complexed to the engineered NK cells measured about 1 hour after incubation and washing, at least about 9% of the antibodies are bound to the NK cell surface at least 3 days after incubation and washing.

192. The composition of claim 190, wherein relative to the amount of antibodies complexed to the engineered NK cells measured about 1 hour after incubation and washing, at least about 6% of the antibodies are bound to the NK cell surface at least 5 days after incubation and washing.

193. The composition of claim 190, wherein relative to the amount of antibodies complexed to the engineered NK cells measured about 1 hour after incubation and washing, at least about 4% of the antibodies are bound to the NK cell surface at least 7 days after incubation and washing.

194. The composition of claim 190, wherein relative to the amount of antibodies complexed to the engineered NK cells measured about 1 hour after incubation and washing, at least about 3% of the antibodies are bound to the NK cell surface at least 12 days after incubation and washing.

195. The composition of any one of claims 163-194, wherein the composition has not been frozen, is cryopreserved, or was thawed from cryopreservation.

196. The composition of claim 195, wherein the composition is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

197. The composition of claim 195 or 196, wherein relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

198. The composition of any one of claims 195-196, wherein the engineered NK cells are deactivated prior to cryopreservation.

199. The composition of claim 198, wherein the deactivating agent comprises a kinase inhibitor.

200. The composition of claim 199, wherein the deactivating agent comprises Dasatinib.

201. The composition of any one of claims 163-186, wherein the composition is produced in vivo.

202. The composition of claim 201, wherein production of the composition in vivo comprises loading the engineered NK cells in vivo with antibody by administering the engineered NK cells within about 1 hour of administration of an antibody.

203. The composition of any one of claims 163-202, wherein the one or more antibody comprises or consists of Amivantamab.

204. The composition of any one of claims 163-203, wherein the one or more antibody comprises or consists of Margetuximab.

205. The composition of any one of claims 163-204, wherein the one or more antibody comprises or consists of Imgatuzumab.

206. The composition of any one of claims 163-205, wherein the one or more antibody comprises or consists of IPH61.

207. A method of producing the composition of any one of claims 163-206, comprising the steps of:
(i) optionally expanding engineered NK cells in a culture comprising an effective amount of:
(a) a cytokine selected from the group consisting of IL-2, IL-15, IL-18, IL-21 and a combination thereof; and
(b) antigen presenting cells/feeders, fragments of antigen presenting cells/feeders, or NK cell-activating beads; and
(ii) providing one or more antibody molecules to the engineered NK cells.

208. The method of claim 207, wherein the method comprises a pre-activating step prior to and/or after the expanding step, wherein the engineered NK cells are pre-activated in a culture comprising an effective concentration of one or more of IL-2, IL-12, IL-15, and IL-18.

209. The method of claim 208, wherein the culture comprises an effective concentration of IL-12, IL-15, and IL-18.

210. The method of any one of claims 207-209, wherein the providing step is further defined as incubating the engineered NK cells with the antibody molecules for a specific duration of time, combining the engineered NK cells and the antibody molecules just prior to infusion, or combining the engineered NK cells and the antibody in vivo in a subject through temporally coordinated administration.

211. The method of claim 210, wherein the duration of time is about 5 minutes to about 24 hours or more.

212. The method of claim 211, wherein the duration of time is about 1 hour.

213. The method of any one of claims 207-212, wherein the culture for the pre-activating step comprises IL-18 and/or IL-15 at a concentration of 0.1-1000 ng/ml, 1-1000 ng/ml, or about 10 ng/mL.

214. The method of any one of claims 207-213, wherein the culture for the pre-activating step comprises IL-12 at a concentration of 0.1-1000 ng/mL, 1-1000 ng/ml, or about 10 ng/mL.

215. The method of any one of claims 207-214, further comprising one or more washing steps of the pre-activated engineered NK cells prior to and/or after the expanding step.

216. The method of any one of claims 207-215, wherein engineered NK cells are activated at least twice or more during the expansion step with IL-12, IL-15, IL-18, IL-2 or any combination thereof.

217. The method of any one of claims 207-216, wherein expanding is for 5-60 days, 12-16 days, or 18-24 days.

218. The method of any one of claims 207-217, wherein the expansion culture further comprises IL-2.

219. The method of claim 218, wherein the IL-2 is present at a concentration of 10-500 U/mL, 100-300 U/mL, or about 200 U/mL.

220. The method of any one of claims 207-219, wherein the IL-2 is replenished in the expansion culture every 2-3 days.

221. The method of any one of claims 207-220, wherein incubating conditions for complexing of the engineered NK cells and one or more antibody are such that at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody for at least 1 hour, 4 hours, 8 hours, 12 hours, 1 day, 2 days, 3 days, 4 days, 5 days, 6 days, 7 days, 8 days, 9 days, 10 days, 11 days, or 12 days following incubation and washing.

222. The method of any one of claims 207-221, wherein relative to the amount of antibodies complexed to the engineered NK cells measured about 1 hour after incubation and washing, at least about 9% of the antibodies are bound to the NK cell surface at least 3 days after incubation and washing.

223. The method of any one of claims 207-222, wherein relative to the amount of antibodies complexed to the engineered NK cells measured about 1 hour after incubation and washing, at least about 6% of the antibodies are bound to the NK cell surface at least 5 days after incubation and washing.

224. The method of any one of claims 207-223, wherein relative to the amount of antibodies complexed to the engineered NK cells measured about 1 hour after incubation and washing, at least about 4% of the antibodies are bound to the NK cell surface at least 7 days after incubation and washing.

225. The method of any one of claims 207-224, wherein relative to the amount of antibodies complexed to the engineered NK cells measured about 1 hour after incubation and washing, at least about 3% of the antibodies are bound to the NK cell surface at least 12 days after incubation and washing.

226. The method of any one of claims 207-225, wherein the NK cells provided with one or more antibodies are not frozen, are cryopreserved, or are thawed from cryopreservation.

227. The method of claim 226, wherein the NK cells provided with one or more antibodies are thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

228. The method of claim 226 or 227, wherein relative to the amount of antibodies complexed to non-cryopreserved engineered NK cells measured about 1 hour after incubation and washing, at least about 30%, 40%, 50%, or 60% of the antibodies are bound to the NK cell surface after thawing from cryopreservation.

229. A method of treating a disease or disorder in a subject, comprising administering a therapeutically effective amount of the compositions of any one of claims 163-205 to the subject.

230. The method of claim 229, wherein the disease or disorder is cancer, inflammation, graft versus host disease, transplant rejection, an autoimmune disorder, an immunodeficiency disease, a B cell malignancy, or an infection.

231. The method of claim 229 or 230, wherein the cancer comprises a hematological cancer or a solid tumor.

232. The method of claim 231, wherein the cancer comprises pancreatic cancer, colorectal cancer, kidney cancer, brain cancer, breast cancer, renal cancer, and/or myeloma.

233. The method of claim 231, wherein the cancer comprises acute myeloid leukemia, blastic plasmacytoid dendritic cell neoplasm, acute lymphoblastic leukemia, hairy cell leukemia, and/or systemic mastocytosis.

234. The method of any one of claims 229-232, wherein the engineered NK cells are allogeneic with respect to the subject.

235. The method of any one of claims 229-232, wherein the engineered NK cells are autologous with respect to the subject.

236. The method of any one of claims 229-235, wherein the subject is a human.

237. The method of any one of claims 229-236, further comprising administering at least a second therapeutic agent to the subject.

238. The method of claim 237, wherein the at least a second therapeutic agent is a therapeutically effective amount of one or more anti-cancer agents, one or more immunomodulatory agents, and/or one or more immunosuppressive agents.

239. The method of claim 238, wherein the anti-cancer agent is chemotherapy, radiotherapy, gene therapy, surgery, hormonal therapy, anti-angiogenic therapy or immunotherapy.

240. The method of claim 238, wherein the immunosuppressive agent is a calcineurin inhibitor, an mTOR inhibitor, an antibody, a chemotherapeutic agent irradiation, a chemokine, an interleukins or an inhibitor of a chemokine or an interleukin.

241. The method of any one of claims 237-240, wherein the composition and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

242. The method of any one of claims 237-241, wherein the second therapeutic agent is an antibody.

243. A method of treating a subject with cancer with an engineered NK cell, the method comprising:
a) optionally stimulating a population of engineered NK cells by pre-activation with a cytokine cocktail;
b) optionally expanding the engineered NK cells; and
ci) loading the engineered NK cells ex vivo with one or more antibody and then administering the loaded engineered NK cells to the subject, or
cii) loading the engineered NK cells in vivo with one or more antibody by administering the engineered NK cells within 1 hour of administration of an antibody;
wherein the engineered NK cell comprises one or more transgenes,
wherein the loading comprises exposure of engineered NK cells to the one or more antibody for an amount of time effective for binding of the antibody to the engineered NK cell surface, and
wherein the one or more transgenes comprise a CD70 targeting CAR comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 2 or 5, and/or a TROP2 targeting CAR comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 7 or 9.

244. The method of claim 243, wherein loading the engineered NK cells comprises exposure of the engineered NK cells to the one or more antibody for greater than or equal to about 1 hour, or less than or equal to about 1 hour.

245. The method of claim 243 or 244, wherein engineered NK cells remain loaded with antibody for at least about or about 1 hour, 4 hours, 8 hours, 12 hours, 1 day, 2 days, 3 days, 4 days, 5 days, 6 days, 7 days, 8 days, 9 days, 10 days, 11 days, or 12 days.

246. The method of any one of claims 243-245, wherein the one or more antibody is bound to the engineered NK cell through interaction with a CD16 surface protein.

247. The method of any one of claims 243-246, wherein the engineered NK cells are derived from cord blood.

248. The method of any one of claims 243-247, wherein the engineered NK cells are activated ex-vivo.

249. The method of any one of claims 243-248, wherein the engineered NK cells are pre-activated with IL-18, IL-15, and IL-12.

250. The method of any one of claims 243-249, wherein the engineered NK cells comprise one or more additional transgenes, optionally comprising an engineered receptor.

251. The method of claim 250, wherein the engineered receptor comprises a chimeric antigen receptor (CAR), a T cell receptor, chemokine receptor, chimeric cytokine receptor, or any combination thereof.

252. The method of claim 250 or 251, wherein the engineered receptor comprises a CAR or a TCR.

253. The method of any one of claims 250-252, wherein the engineered receptor targets a stem cell antigen, auto-antigen, or a cancer antigen selected from the group consisting of CD19, CD319 (CS1), ROR1, CD20, CD22, CD70, carcinoembryonic antigen, alphafetoprotein, CA-125, MUC-1, EGFR, epithelial tumor antigen, melanoma-associated antigen, mutated p53, mutated ras, EGFR-2/HER2/Neu, ERBB2, folate binding protein, HIV-1 envelope glycoprotein gp120, HIV-1 envelope glycoprotein gp41, GD2, CD5, CD123, CD23, CD30, CD38, CD56, CD70, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, HLA-G, Trop2, and a combination thereof.

254. The method of any one of claims 243-253, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 1, 3, or 4.

255. The method of any one of claims 243-254, wherein the engineered NK cell comprises a construct at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 6, 8, or 10.

256. The method of any one of claims 243-255, wherein the one or more transgenes comprise a cytokine.

257. The method of claim 256 wherein the one or more heterologous cytokine comprises IL-2, IL-4, IL-7, IL-12, IL-15, IL-18, IL-21 and/or IL-23.

258. The method of claim 257, wherein the cytokine comprises IL-2, IL-12, IL-15, IL-18, and/or IL-21.

259. The method of claim 258, wherein a cytokine comprises or consists of IL-15 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 11 or 12.

260. The method of claim 258 or 259, wherein a cytokine comprises or consists of IL-21 encoded by and/or comprising a sequence at least or exactly 80%, 81%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NOs: 13-16.

261. The method of any one of claims 243-260, wherein the engineered NK cells comprise on or more engineered mutations in an endogenous gene.

262. The method of claim 261, wherein the one or more engineered mutation in an endogenous gene comprises a CISH, CD38, Glucocorticoid Receptor, and/or TGFBR2 gene mutation.
